SEQUENCE LISTING (64 sequences)

SEQ ID NO: 1 - protein, 118 aa, Artificial Sequence
  Description of Artificial Sequence Synthetic polypeptide
QIQLVQSGPELKKPGETVKISCKASGYTFTDYSMHWMKQAPGKGLKWMVWINTETGEPTYADDFKGRFAFSLETSANTAYLKINNLKNEDTATYFCARGGIFYGMDYWGQGTSVTVSS

SEQ ID NO: 2 - protein, 108 aa, Artificial Sequence
  Description of Artificial Sequence Synthetic polypeptide
SIVMTQTPKFLLISAGDRVTITCKASQSVINDAAWYQQKPGQSPRLLIYYTSHRYTGVPDRFTGSGYGTDFTFTISTVQAEDLAVYFCQQDYTSPWTFGGGTKLEIKR

SEQ ID NO: 3 - protein, 118 aa, Artificial Sequence
  Description of Artificial Sequence Synthetic polypeptide
QVQLVQSGAEVKKPGASVKVSCKASGYTFTDYSMHWVRQAPGQGLEWMVWINTETGEPTYADDFKGRVTITRDTSTSTAYLELSSLRSEDTAVYYCARGGIFYGMDYWGQGTLVTVSS

SEQ ID NO: 4 - protein, 118 aa, Artificial Sequence
  Description of Artificial Sequence Synthetic polypeptide
QVQLVQSGAEVKKPGASVKVSCKASGYTFTDYSMHWVRQAPGQGLEWMVWINTETGEPTYADDFKGRVTITLDTSTSTAYLELSSLRSEDTAVYYCARGGIFYGMDYWGQGTLVTVSS

SEQ ID NO: 5 - protein, 118 aa, Artificial Sequence
  Description of Artificial Sequence Synthetic polypeptide
QVQLVQSGAEVKKPGASVKVSCKASGYTFTDYSMHWVRQAPGQGLEWMVWINTETGEPTYADDFKGRFTFTLDTSTSTAYLELSSLRSEDTAVYYCARGGIFYGMDYWGQGTLVTVSS

SEQ ID NO: 6 - protein, 118 aa, Artificial Sequence
  Description of Artificial Sequence Synthetic polypeptide
EVQLVESGGGLVQPGGSLRLSCAASGYTFTDYSMHWVRQAPGKGLEWVVWINTETGEPTYADDFKGRFTISRDNSKNTLYLQMNSLRAEDTAVYYCARGGIFYGMDYWGQGTLVTVSS

SEQ ID NO: 7 - protein, 118 aa, Artificial Sequence
  Description of Artificial Sequence Synthetic polypeptide
EVQLVESGGGLVQPGGSLRLSCAASGYTFTDYSMHWVRQAPGKGLEWVVWINTETGEPTYADDFKGRFTFSLDNSKNTAYLQMNSLRAEDTAVYYCARGGIFYGMDYWGQGTLVTVSS

SEQ ID NO: 8 - protein, 118 aa, Artificial Sequence
  Description of Artificial Sequence Synthetic polypeptide
EVQLVESGGGLVQPGGSLRLSCAASGYTFTDYSMHWVRQAPGKGLEWVVWINTETGEPTYADDFKGRFTISLDNSKNTAYLQMNSLRAEDTAVYYCARGGIFYGMDYWGQGTLVTVSS

SEQ ID NO: 9 - protein, 118 aa, Artificial Sequence
  Description of Artificial Sequence Synthetic polypeptide
EVQLVESGGGLVQPGGSLRLSCAASGYTFTDYSMHWVRQAPGKGLEWVVWINTETGEPTYADSVKGRFTISLDNSKNTAYLQMNSLRAEDTAVYYCARGGIFYGMDYWGQGTLVTVSS

SEQ ID NO: 10 - protein, 108 aa, Artificial Sequence
  Description of Artificial Sequence Synthetic polypeptide
DIQMTQSPSSLSASVGDRVTITCKASQSVINDAAWYQQKPGKAPKLLIYYTSHRYTGVPSRFSGSGSGTDFTLTISSLQPEDFATYYCQQDYTSPWTFGQGTKVEIKR

SEQ ID NO: 11 - protein, 108 aa, Artificial Sequence
  Description of Artificial Sequence Synthetic polypeptide
EIVLTQSPATLSLSPGERATLSCKASQSVINDAAWYQQKPGQAPRLLIYYTSHRYTGIPARFSGSGSGTDFTLTISSLEPEDFAVYYCQQDYTSPWTFGQGTKVEIKR

SEQ ID NO: 12 - protein, 10 aa, Artificial Sequence
  Description of Artificial Sequence Synthetic peptide
GYTFTDYSMH

SEQ ID NO: 13 - protein, 18 aa, Artificial Sequence
  Description of Artificial Sequence Synthetic peptide
VWINTETGEPTYADSVKG

SEQ ID NO: 14 - protein, 9 aa, Artificial Sequence
  Description of Artificial Sequence Synthetic peptide
GGIFYGMDY

SEQ ID NO: 15 - protein, 11 aa, Artificial Sequence
  Description of Artificial Sequence Synthetic peptide
KASQSVINDAA

SEQ ID NO: 16 - protein, 7 aa, Artificial Sequence
  Description of Artificial Sequence Synthetic peptide
YTSHRYT

SEQ ID NO: 17 - protein, 9 aa, Artificial Sequence
  Description of Artificial Sequence Synthetic peptide
QQDYTSPWT

SEQ ID NO: 18 - protein, 18 aa, Artificial Sequence
  Description of Artificial Sequence Synthetic peptide
VWINTETGEPTYADDFKG

SEQ ID NO: 19 - protein, 118 aa, Artificial Sequence
  Description of Artificial Sequence Synthetic polypeptide
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 20 - protein, 112 aa, Artificial Sequence
  Description of Artificial Sequence Synthetic polypeptide
DIVLTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 21 - protein, 11 aa, Artificial Sequence
  Description of Artificial Sequence Synthetic peptide
GFSLSAYSVNW

SEQ ID NO: 22 - protein, 16 aa, Artificial Sequence
  Description of Artificial Sequence Synthetic peptide
MIWGDGKIVYNSALKS

SEQ ID NO: 23 - protein, 10 aa, Artificial Sequence
  Description of Artificial Sequence Synthetic peptide
DGYYPYAMDN

SEQ ID NO: 24 - protein, 15 aa, Artificial Sequence
  Description of Artificial Sequence Synthetic peptide
RASKSVDSYGNSFMH

SEQ ID NO: 25 - protein, 7 aa, Artificial Sequence
  Description of Artificial Sequence Synthetic peptide
LASNLES

SEQ ID NO: 26 - protein, 9 aa, Artificial Sequence
  Description of Artificial Sequence Synthetic peptide
QQNNEDPRT

SEQ ID NO: 27 - protein, 153 aa, Homo sapiens [sequence omitted]

SEQ ID NO: 28 - protein, 129 aa, Homo sapiens [sequence omitted]

SEQ ID NO: 29 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 30 - protein, 114 aa, Homo sapiens
SPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 31 - protein, 122 aa, Homo sapiens
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGQFN

SEQ ID NO: 32 - protein, 132 aa, Macaca fascicularis
MALLLTMVIALTCLGGFASPSPVPPSTALKELIEELVNITQNQKAPLCNGSMVWSINLTAGVYCAALESLINVSGCSAIEKTQRMLNGFCPHKVSAGQFSSLRVRDTKIEVAQFVKDLLVHLKKLFREGQFN

SEQ ID NO: 33 - protein, 153 aa, Macaca fascicularis
MGLTSQLLPPLFFLLACAGNFVHGHKCDITLQEIIKTLNSLTEQKTLCTKLTITDILAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 34 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 327 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 327 aa, Artificial Sequence
  Description of Artificial Sequence Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 38 - protein, 445 aa, Artificial Sequence
  Description of Artificial Sequence Synthetic polypeptide
EVQLVESGGGLVQPGGSLRLSCAASGYTFTDYSMHWVRQAPGKGLEWVVWINTETGEPTYADSVKGRFTISLDNSKNTAYLQMNSLRAEDTAVYYCARGGIFYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 39 - protein, 214 aa, Artificial Sequence
  Description of Artificial Sequence Synthetic polypeptide
DIQMTQSPSSLSASVGDRVTITCKASQSVINDAAWYQQKPGKAPKLLIYYTSHRYTGVPSRFSGSGSGTDFTLTISSLQPEDFATYYCQQDYTSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 445 aa, Artificial Sequence
  Description of Artificial Sequence Synthetic polypeptide
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 218 aa, Artificial Sequence
  Description of Artificial Sequence Synthetic polypeptide
DIVLTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 8 aa, Homo sapiens
ESLINVSG

SEQ ID NO: 43 - protein, 12 aa, Homo sapiens
YCAALESLINVS

SEQ ID NO: 44 - protein, 106 aa, Artificial Sequence
  Description of Artificial Sequence Synthetic polypeptide
DIVLTQSPASLAVSLGQRATISCRASQSVSTSSYSYMNWYQQTPGQPPKLLIKYASNLESGIPARFSGSGSGTDFTLNIHPVEEEDTATYYCQHSWEIPYTFGGGT

SEQ ID NO: 45 - protein, 123 aa, Artificial Sequence
  Description of Artificial Sequence Synthetic polypeptide
QVTLKESGPGILQPSQTLSLTCSFSGFSLSTSDMGVGWIRQPSGKGLEWLAHIWWDDVKRYNPALKSRLTISKDTSSSQVFLKIASVDTADTATYYCARIGTNYGYDGLFDYWGQGTTLTVSS

SEQ ID NO: 46 - protein, 218 aa, Artificial Sequence
  Description of Artificial Sequence Synthetic polypeptide
DIVMTQSPDSLAVSLGERATINCRASQSVSTSSYSYMNWYQQKPGQPPKLLIKYASNLESGVPDRFSGSGSGTDFTLTISSLQAEDVAVYYCQHSWEIPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 453 aa, Artificial Sequence
  Description of Artificial Sequence Synthetic polypeptide
EVQLVESGPALVKPTQTLTLTCTFSGFSLSTSDMGVGWIRQPPGKALEWLAHIWWDDVKRYNPALKSRLTISKDTSKNQVVLTMTNMDPVDTATYYCARIGTNYGYDALFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 111 aa, Artificial Sequence
  Description of Artificial Sequence Synthetic polypeptide
DIVMTQSPDSLAVSLGERATINCRASQSVSTSSYSYMNWYQQKPGQPPKLLIKYASNLESGVPDRFSGSGSGTDFTLTISSLQAEDVAVYYCQHSWEIPYTFGQGTKVEIK

SEQ ID NO: 49 - protein, 123 aa, Artificial Sequence
  Description of Artificial Sequence Synthetic polypeptide
EVQLVESGPALVKPTQTLTLTCTFSGFSLSTSDMGVGWIRQPPGKALEWLAHIWWDDVKRYNPALKSRLTISKDTSKNQVVLTMTNMDPVDTATYYCARIGTNYGYDALFDYWGQGTLVTVSS

SEQ ID NO: 50 - protein, 12 aa, Artificial Sequence
  Description of Artificial Sequence Synthetic peptide
GFSLSTSDMGVG

SEQ ID NO: 51 - protein, 17 aa, Artificial Sequence
  Description of Artificial Sequence Synthetic peptide
AHIWWDDVKRYNPALKS

SEQ ID NO: 52 - protein, 15 aa, Artificial Sequence
  Description of Artificial Sequence Synthetic peptide
ARIGTNYGYDALFDY

SEQ ID NO: 53 - protein, 15 aa, Artificial Sequence
  Description of Artificial Sequence Synthetic peptide
RASQSVSTSSYSYMN

SEQ ID NO: 54 - protein, 7 aa, Artificial Sequence
  Description of Artificial Sequence Synthetic peptide
YASNLES

SEQ ID NO: 55 - protein, 9 aa, Artificial Sequence
  Description of Artificial Sequence Synthetic peptide
QHSWEIPYT

SEQ ID NO: 56 - protein, 118 aa, Artificial Sequence [sequence omitted]

SEQ ID NO: 57 - protein, 112 aa, Artificial Sequence
  Description of Artificial Sequence Synthetic polypeptide
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKR

SEQ ID NO: 58 - protein, 445 aa, Artificial Sequence
  Description of Artificial Sequence Synthetic polypeptide
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 218 aa, Artificial Sequence
  Description of Artificial Sequence Synthetic polypeptide
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 5 aa, Artificial Sequence
  Description of Artificial Sequence Synthetic peptide
AYSVN

SEQ ID NO: 61 - protein, 107 aa, Artificial Sequence
  Description of Artificial Sequence Synthetic polypeptide
DIQMTQSPSSLSASVGDRVTITCRASQGISSYLAWYQQKPGKAPKLLIYAASSLQSGVPSRFSGSGSGTDFTLTISSLQPEDFATYYCQQYNSYPTFGQGTKVEIKR

SEQ ID NO: 62 - protein, 107 aa, Artificial Sequence
  Description of Artificial Sequence Synthetic polypeptide
EIVLTQSPATLSLSPGERATLSCRASQSVSSYLAWYQQKPGQAPRLLIYGASTRATGIPARFSGSGSGTDFTLTISSLEPEDFAVYYCQQYNNWPTFGQGTKVEIKR

SEQ ID NO: 63 - protein, 109 aa, Artificial Sequence
  Description of Artificial Sequence Synthetic polypeptide
QVQLVQSGAEVKKPGASVKVSCKASGYTFTSYYMHWVRQAPGQGLEWMGWINPGNGNTNYAQKFQGRVTITRDTSTSTAYMELSSLRSEDTAVYYCARWGQGTLVTVSS

SEQ ID NO: 64 - protein, 110 aa, Artificial Sequence
  Description of Artificial Sequence Synthetic polypeptide
EVQLVESGGGLVQPGGSLRLSCAASGFTFSSYAMSWVRQAPGKGLEWVSVISSKGGSTYYADSVKGRFTISRDNSKNTLYLQMNSLRAEDTAVYYCARGWGQGTLVTVSS

(19) United States
(12) Patent Application Publication
(10) Pub. No.: US 2016/0207995 A1
(43) Pub. Date: Jul. 21, 2016

(54) ANTI-IL-4 ANTIBODIES AND BISPECIFIC ANTIBODIES AND USES THEREOF

(71) Applicant: Genentech, Inc., South San Francisco, CA (US)

(72) Inventors: Daniel G. Yansura, Pacifica, CA (US); Nancy Y. Chiang, San Francisco, CA (US); Mark S. Dennis, San Carlos, CA (US); Michael Dillon, San Francisco, CA (US); Germaine G. Fuh, Pacifica, CA (US); Gerald R. Nakamura, San Francisco, CA (US); Christoph Spiess, Mountain View, CA (US); Lawren C. Wu, Foster City, CA (US); Yin Zhang, Fremont, CA (US)

(73) Assignee: GENENTECH, INC., South San Francisco, CA (US)

(21) Appl. No.: 14/858,251

(22) Filed: Sep. 18, 2015

Related U.S. Application Data
(63) Continuation of application No. PCT/US2014/032998, filed on Apr. 4, 2014.
(60) Provisional application No. 61/808,748, filed on Apr. 5, 2013.

(51) Int. Cl. C07K 16/24; A61K 45/06; A61K 39/395

(52) U.S. Cl. CPC C07K 16/247; C07K 16/244; A61K 39/3955; A61K 45/06; C07K 2317/31; C07K 2317/76; C07K 2317/56; C07K 2317/51; C07K 2317/24; C07K 2317/14; C07K 2317/92; A61K 2039/505

(57) ABSTRACT

The invention provides anti-IL-4 antibodies and bispecific antibodies and methods of using the same.

### CROSS REFERENCE TO RELATED APPLICATION

This application is a continuation of International Application No. PCT/US2014/032998 having an international filing date of Apr. 4, 2014, which claims the benefit of priority of provisional U.S. Application No. 61/808,748 filed Apr. 5, 2013, both of which are hereby incorporated by reference in their entirety.

### SEQUENCE LISTING

The instant application contains a Sequence Listing which has been submitted via EFS-Web and is hereby incorporated by reference in its entirety. Said ASCII copy, created on Mar. 12, 2014, is named 2014.MAR.12 P5609R1-WO_SL and is 75,442 bytes in size.

### FIELD

The present invention relates to anti-IL-4 antibodies and bispecific antibodies and methods of using the same.

### BACKGROUND

Asthma is a complex disease with increasing worldwide incidence. Among other events, eosinophilic inflammation has been reported in the airways of asthma patients. The pathophysiology of the disease is characterized by variable airflow obstruction, airway inflammation, mucus hypersecretion, and subepithelial fibrosis. Clinically, patients may present with cough, wheezing, and shortness of breath. While many patients are adequately treated with currently available therapies, some patients with asthma have persistent disease despite the use of current therapies.

A number of studies have implicated IL-4, IL-13, and their receptors in the pathogenesis of asthma and allergy (see, e.g., Wills-Karp, 2004, Immunol. Rev. 202, 175-190; Brightling et al., 2010, Clin. Exp. Allergy 40, 42-49; Finkelman et al., 2010, J Immunol 184, 1663-1674; Maes et al., 2012, Am. J. Respir. Cell Mol. Biol. 47, 261-270; Steinke and Borish, 2001, Respir. Res. 2, 66-70). IL-4 binds to two receptors, one a heterodimer of IL-4Rα and the common gamma chain (γc), and the other a heterodimer of IL-4 receptor alpha (IL-4Rα) and IL-13 receptor alpha 1 (IL-13Rα1). The latter receptor IL-4Rα/IL-13Rα1 is a shared receptor with IL-13, which also uniquely binds a single chain receptor consisting of IL-13 receptor alpha 2 (IL-13Rα2). Polymorphisms of the IL-4, IL-13, and IL-4Rα genes are associated with asthma and allergy, including features such as IgE levels, prevalence of atopy, and severity of asthma disease. In addition, expression of IL-4, IL-13, and their receptors are increased in asthma and other allergic diseases. Moreover, neutralization or deficiency of IL-4, IL-13, and their receptors ameliorates disease in preclinical models of asthma.

A number of drugs are on the market or in development for treating asthma. One of the numerous targets for asthma therapy is IL-13. IL-13 is a pleiotropic TH2 cytokine produced by activated T cells, NKT cells, basophils, eosinophils, and mast cells, and it has been strongly implicated in the pathogenesis of asthma in preclinical models. IL-13 antagonists, including anti-IL-13 antibodies, have previously been described. See, e.g., Intn'l Patent Application Pub. No. WO 2005/062967. Such antibodies have also been developed as human therapeutics. Recently, several studies have shown clinical activity of monoclonal antibodies against IL-13 in the treatment of asthma (See, e.g., Corren et al., 2011, N. Engl. J. Med. 365, 1088-1098; Gauvreau et al., 2011, Am. J. Respir. Crit. Care Med. 183, 1007-1014; Ingram and Kraft, 2012, J. Allergy Clin. Immunol. 130, 829-42; Webb, 2011, Nat Biotechnol 29, 860-863). Of these, lebrikizumab, a humanized IgG4 antibody that neutralizes IL-13 activity, improved lung function in asthmatics who were symptomatic despite treatment with, for the majority, inhaled corticosteroids and a long-acting beta2-adrenergic receptor agonist (Corren et al., 2011, N. Engl. J. Med. 365, 1088-1098). In addition, a bispecific antibody that binds IL-13 and IL-4 has been described. See, e.g., U.S. Publication No. 2010/0226923.

Yet moderate to severe asthmatic patients are still in need of alternative treatment options. Thus, there is a need to identify better therapies for treating asthma and improved methods for understanding how to treat asthma patients.

Idiopathic pulmonary fibrosis (IPF) is a restrictive lung disease characterized by progressive interstitial fibrosis of lung parenchyma, affecting approximately 100,000 patients in the United States (Raghu et al., Am J Respir Crit Care Med 174:810-816 (2006)). This interstitial fibrosis associated with IPF leads to progressive loss of lung function, resulting in death due to respiratory failure in most patients. The median survival from the time of diagnosis is 2-3 years (Raghu et al., Am J Respir Crit Care Med 183:788-824 (2011)). The etiology and key molecular and pathophysiological drivers of IPF are unknown. The only treatment shown to prolong survival in IPF patients is lung transplantation (Thabut et al., Annals of internal medicine 151:767-774 (2009)). Lung transplantation, however, is associated with considerable morbidity, not all IPF patients are appropriate candidates for it, and there is a relative paucity of suitable donor lungs. Despite numerous attempts, no drug therapies to date have been shown to substantially prolong survival in a randomized, placebo-controlled interventional trial in IPF patients, although some interventions have appeared to slow the rate of lung function decline in some patients (Raghu et al., Am J Respir Crit Care Med 183:788-824 (2011); Richeldi et al., The New England J. of Med. 365:1079-1087 (2011)).

IL-4 and IL-13 signaling can induce fibrogenic responses from a number of cell types in vitro. Treatment of fibroblasts with IL-4 or IL-13 has been shown to induce collagen production and differentiation to a myofibroblast phenotype (Borowski et al., J. British Soc. Allergy Clin. Immunol., 38: 619-628 (2008); Hashimoto et al., J. Allergy Clin. Immunol., 107: 1001-1008 (2001); Murray, et al., Int. J. Biochem. Cell Biol., 40: 2174-2182 (2008); Saito et al., Intl. Archives Allergy Immunol., 132: 168-176 (2003)). Alternatively activated macrophages have also been proposed to be major contributors to fibrogenic processes, in part based on their ability to produce growth factors, such as TGFβ and PDGF, that stimulate fibroblasts and myofibroblasts. IL-4 and IL-13 are potent inducers of the alternatively activated macrophage phenotype and may drive fibrogenic responses at least partially through its activity on these cells (Doyle et al., Eur. J. Immunol., 24: 1441-1445 (1994); Song et al., Cell. Immunol., 204: 19-28 (2000); Wynn and Barron, Seminars Liver Dis., 30: 245-257 (2010).

IL-4 and IL-13 can also drive fibrogenic responses in multiple tissues in vivo. Transgenic overexpression of IL-4 or IL-13 in the lungs of mice is sufficient to induce collagen gene expression and profound sub-epithelial fibrosis (Lee et al., J. Exper. Med., 194: 890-821 (2001); Ma et al. J. Clin. Invest., 116: 1274-1283 (2006); Zhu et al., J. Clin. Invest. 103: 779-788 (1999)). Additionally, a number of studies have demonstrated a role for IL-4 and IL-13 as drivers of fibrosis in pre-clinical animal models. Mice with targeted disruption of IL-13 or that are treated with blocking antibodies specific for IL-13 show reduced extracellular matrix deposition in Bleomycin- and FITC-induced pulmonary fibrosis models (Belperio et al., Am. J. Respir. Cell Mol. Biol., 27: 419-427 (2002); Kolodsick et al., J. Immunol., 172: 4068-4076 (2004); Liu et al., J. Immunol., 173: 3425-3431 (2004)). Similarly, IL-4 has been shown to be important in sustaining fibrotic responses in the Bleomycin-induced pulmonary fibrosis model (Huaux et al., J. Immunol., 170: 2083-2092 (2003).

Multiple studies have concluded that expression and activity of IL-4 and/or IL-13 is elevated in IPF patients. The expression of IL-4, IL-13 and IL-4/IL-13 receptor subunits were found to be increased in lung biopsy samples from IPF patients compared to normal controls, both at the level of mRNA and protein (Jakubziak et al., J. Clin. Pathol., 57: 477-486 (2004)). Notably, in this study IL-13Rα2, a gene that is highly induced by IL-4 or IL-13 signaling (David et al., Oncogene, 22: 2286-3394 (2003)), was found to be expressed in fibroblastic foci in IPF biopsies by immunohistochemistry, suggesting active IL-4 or IL-13 signaling in these cells. IL-4 and IL-13 were also found to be elevated in bronchoalveolar lavage fluid of IPF patients compared to normal controls. Notably, the level of IL-13 in these samples negatively correlated with the key measures of lung function, percent predicted FVC and DLCO (Park et al., J. Korean Med. Sci., 24: 614-620 (2009)), suggesting pathogenic functions of IL-13 in IPF patients.

IPF patients are still in need of alternative treatment options. Thus, there is a need to identify better therapies for treating IPF and improved methods for understanding how to treat IPF patients

All references cited herein, including patent applications and publications, are incorporated by reference herein in their entirety for any purpose.

### SUMMARY

In some embodiments, a multispecific antibody is provided, wherein the multispecific antibody comprises an antigen-binding domain that comprises a first VH/VL unit that specifically binds IL-4 and a second VH/VL unit that specifically binds IL-13. In some embodiments, the multispecific antibody:
a) inhibits binding of IL-4 to IL-4 receptor alpha (IL-4Rα),
b) inhibits IL-4-induced proliferation of cells in vitro, and/or
b) inhibits IL-13-induced proliferation of cells in vitro.

In some embodiments, the first VH/VL unit of the multispecific antibody comprises HVR-H3 comprising the amino acid sequence of SEQ ID NO: 14, HVR-L3 comprising the amino acid sequence of SEQ ID NO: 17, and HVR-H2 comprising the amino acid sequence of SEQ ID NO: 13 or SEQ ID NO: 18. In some embodiments, the first VH/VL unit comprises HVR-H1 comprising the amino acid sequence of SEQ ID NO: 12, HVR-H2 comprising the amino acid sequence of SEQ ID NO: 13 or SEQ ID NO: 18, and HVR-H3 comprising the amino acid sequence of SEQ ID NO: 14. In some embodiments, the first VH/VL unit comprises HVR-L1 comprising the amino acid sequence of SEQ ID NO: 15, HVR-L2 comprising the amino acid sequence of SEQ ID NO: 16, and HVR-L3 comprising the amino acid sequence of SEQ ID NO: 17. In some embodiments, the first VH/VL unit comprises (a) a VH sequence having at least 95% sequence identity to the amino acid sequence of SEQ ID NO: 9; (b) a VL sequence having at least 95% sequence identity to the amino acid sequence of SEQ ID NO: 10; or (c) a VH sequence as in (a) and a VL sequence as in (b). In some embodiments, the first VH/VL unit comprises a VH sequence selected from SEQ ID NOs: 1 and 3 to 9. In some embodiments, the first VH/VL unit comprises a VL sequence selected from SEQ ID NOs: 2, 10, and 11. In some embodiments, the first VH/VL unit comprises the VH sequence of SEQ ID NO: 9 and the VL sequence of SEQ ID NO: 10.

In any of the embodiments described herein, the second VH/VL unit of the multispecific antibody may comprise: (a) HVR-H3 comprising the amino acid sequence of SEQ ID NO: 23, HVR-L3 comprising the amino acid sequence of SEQ ID NO: 26, and HVR-H2 comprising the amino acid sequence of SEQ ID NO: 22; or (b) HVR-H3 comprising the amino acid sequence of SEQ ID NO: 52, HVR-L3 comprising the amino acid sequence of SEQ ID NO: 55, and HVR-H2 comprising the amino acid sequence of SEQ ID NO: 51. In any of the embodiments described herein, the second VH/VL unit of the multispecific antibody may comprise: (a) HVR-H1 comprising the amino acid sequence of SEQ ID NO: 21 or the amino acid sequence of SEQ ID NO: 60, HVR-H2 comprising the amino acid sequence of SEQ ID NO: 22, and HVR-H3 comprising the amino acid sequence of SEQ ID NO: 23; or (b) HVR-H1 comprising the amino acid sequence of SEQ ID NO: 50, HVR-H2 comprising the amino acid sequence of SEQ ID NO: 51, and HVR-H3 comprising the amino acid sequence of SEQ ID NO: 52. In any of the embodiments described herein, the second VH/VL unit of the multispecific antibody may comprise: (a) HVR-L1 comprising the amino acid sequence of SEQ ID NO: 24, HVR-L2 comprising the amino acid sequence of SEQ ID NO: 25, and HVR-L3 comprising the amino acid sequence of SEQ ID NO: 26; or (b) HVR-L1 comprising the amino acid sequence of SEQ ID NO: 53, HVR-L2 comprising the amino acid sequence of SEQ ID NO: 54, and HVR-L3 comprising the amino acid sequence of SEQ ID NO: 55. In any of the embodiments described herein, the second VH/VL unit of the multispecific antibody may comprise: (a) a VH sequence having at least 95% sequence identity to the amino acid sequence of SEQ ID NO: 19; (b) a VL sequence having at least 95% sequence identity to the amino acid sequence of SEQ ID NO: 20; (c) a VH sequence as in (a) and a VL sequence as in (b); (d) a VH sequence having at least 95% sequence identity to the amino acid sequence of SEQ ID NO: 49; (e) a VL sequence having at least 95% sequence identity to the amino acid sequence of SEQ ID NO: 48; or (f) a VH sequence as in (d) and a VL sequence as in (e). In any of the embodiments described herein, the second VH/VL unit of the multispecific antibody may comprise the VH sequence of SEQ ID NO: 19, 56, or 49. In any of the embodiments described herein, the second VH/VL unit of the multispecific antibody may comprise the VL sequence of SEQ ID NO: 20, 57, or 48. In any of the embodiments described herein, the second VH/VL unit of the multispecific antibody may comprise the VH sequence of SEQ ID NO: 19 or 56 and the VL sequence of SEQ ID NO: 20 or 57; or the VH sequence of SEQ ID NO: 49 and the VL sequence of SEQ ID NO: 48.

In some embodiments, the multispecific antibody competes for binding to IL-4 with an antibody comprising a VH sequence of SEQ ID NO: 9 and a VL sequence of SEQ ID NO: 10. In some embodiments, the multispecific antibody competes for binding to IL-13 with an antibody comprising a VH sequence of SEQ ID NO: 19 and a VL sequence of SEQ ID NO: 20, or with an antibody comprising a VH sequence of SEQ ID NO: 49 and a VL sequence of SEQ ID NO: 48. In some embodiments, the multispecific antibody binds an epitope within amino acids 77 to 89 of SEQ ID NO: 29, or within amino acids 82 to 89 of SEQ ID NO: 29.

In some embodiments, a multispecific antibody is provided that comprises a first VH/VL unit that specifically binds IL-4 and a second VH/VL unit that specifically binds IL-13, wherein the first VH/VL unit comprises the VH sequence of SEQ ID NO: 9 and the VL sequence of SEQ ID NO: 10, and the second VH/VL unit comprises the VH sequence of SEQ ID NO: 19 and the VL sequence of SEQ ID NO: 20.

In any of the embodiments described herein, the multispecific antibody may be an IgG antibody. In any of the embodiments described herein, the multispecific antibody may be an IgG1 or IgG4 antibody. In any of the embodiments described herein, the multispecific antibody may be an IgG4 antibody.

In any of the embodiments described herein, the multispecific antibody may comprise a first heavy chain constant region and a second heavy chain constant region, wherein the first heavy chain constant region comprises a knob mutation and the second heavy chain constant region comprises a hole mutation. In some embodiments, the first heavy chain constant region is fused to the heavy chain variable region portion of a VH/VL unit that binds IL-4. In some embodiments, the second heavy chain constant region is fused to the heavy chain variable region portion of a VH/VL unit that binds IL-13. In some embodiments, the first heavy chain constant region is fused to the heavy chain variable region portion of a VH/VL unit that binds IL-13. In some embodiments, the second heavy chain constant region is fused to the heavy chain variable region portion of a VH/VL unit that binds IL-4.

In some embodiments, the multispecific antibody is an IgG1 antibody comprising a knob mutation that comprises a T366W mutation. In some embodiments, the multispecific antibody is an IgG1 antibody comprising a hole mutation that comprises at least one, at least two, or three mutations selected from T366S, L368A, and Y407V. In some embodiments, the multispecific antibody is an IgG4 antibody comprising a knob mutation that comprises a T366W mutation. In some embodiments, the multispecific antibody is an IgG4 antibody comprising a hole mutation that comprises at least one, at least two, or three mutations selected from T366S, L368A, and Y407V. In some embodiments, the multispecific antibody comprises a first heavy chain constant region comprising the sequence of SEQ ID NO: 34 or SEQ ID NO: 36. In some embodiments, the multispecific antibody comprises a second heavy chain constant region comprising the sequence of SEQ ID NO: 35 or SEQ ID NO: 37.

In some embodiments, a multispecific antibody is provided, wherein the antibody comprises a first heavy chain comprising the sequence of SEQ ID NO: 38, a first light chain comprising the sequence of SEQ ID NO: 39, a second heavy chain comprising the sequence of SEQ ID NO: 40, and a second light chain comprising the sequence of SEQ ID NO: 41.

In some embodiments, isolated antibodies that bind to IL-4 are provided. In some embodiments, the antibody comprises: (a) HVR-H3 comprising the amino acid sequence of SEQ ID NO: 14, HVR-L3 comprising the amino acid sequence of SEQ ID NO: 17, and HVR-H2 comprising the amino acid sequence of SEQ ID NO: 13 or SEQ ID NO: 18; or (b) HVR-H1 comprising the amino acid sequence of SEQ ID NO: 12, HVR-H2 comprising the amino acid sequence of SEQ ID NO: 13 or SEQ ID NO: 18, and HVR-H3 comprising the amino acid sequence of SEQ ID NO: 14; or (c) HVR-L1 comprising the amino acid sequence of SEQ ID NO: 15, HVR-L2 comprising the amino acid sequence of SEQ ID NO: 16, and HVR-L3 comprising the amino acid sequence of SEQ ID NO: 17; or (d) a VH sequence having at least 95% sequence identity to the amino acid sequence of SEQ ID NO: 9; or (e) a VL sequence having at least 95% sequence identity to the amino acid sequence of SEQ ID NO: 10. In some embodiments, the antibody comprises HVR-H1 comprising the amino acid sequence of SEQ ID NO: 12, HVR-H2 comprising the amino acid sequence of SEQ ID NO: 13 or SEQ ID NO: 18, HVR-H3 comprising the amino acid sequence of SEQ ID NO: 14, HVR-L1 comprising the amino acid sequence of SEQ ID NO: 15, HVR-L2 comprising the amino acid sequence of SEQ ID NO: 16, and HVR-L3 comprising the amino acid sequence of SEQ ID NO: 17. In some embodiments, the antibody comprises a VH sequence having at least 95% sequence identity to the amino acid sequence of SEQ ID NO: 9 and a VL sequence having at least 95% sequence identity to the amino acid sequence of SEQ ID NO: 10. In some embodiments, the antibody comprises a VH sequence selected from SEQ ID NOs: 1 and 3 to 9. In some embodiments, the antibody comprises a VL sequence selected from SEQ ID NOs: 2, 10, and 11.

In some embodiments, an isolated antibody that binds to IL-4 is provided, wherein the antibody comprises the VH sequence of SEQ ID NO: 9 and the VL sequence of SEQ ID NO: 10.

In some embodiments, an isolated nucleic acid is provided that encodes any of the bispecific antibodies or isolated antibodies described herein. In some embodiments, an isolated nucleic acid is provided that encodes a first VH/VL unit of any of the multispecific antibodies described herein. In some embodiments, an isolated nucleic acid is provided that encodes a second VH/VL unit of any of the multispecific antibodies described herein. In some embodiments, a host cell is provided that comprises the isolated nucleic acid. In some embodiments, the host cell is an E. coli cell or a CHO cell. In some embodiments, a method of producing an antibody is provided comprising culturing the host cell.

In some embodiments, an immunoconjugate is provided, wherein the immunoconjugate comprises any of the multispecific antibodies or isolated antibodies described herein and a cytotoxic agent.

In some embodiments, pharmaceutical formulations are provided, comprising any of the multispecific antibodies or isolated antibodies described herein and a pharmaceutically acceptable carrier.

In some embodiments, the antibodies described herein are provided for use as a medicament. In some embodiments, the antibodies described herein are provided for use in treating an eosinophilic disorder, an IL-13 mediated disorder, an IL-4 mediated disorder, or a respiratory disorder. In some embodiments, use of the antibodies described herein in the manufacture of a medicament for treating an eosinophilic disorder, an IL-13 mediated disorder, an IL-4 mediated disorder, or a respiratory disorder is provided. In some embodiments, methods of treating an eosinophilic disorder, an IL-13 mediated disorder, an IL-4 mediated disorder, or a respiratory disorder in an individual are provided comprising administering to the individual an effective amount of an antibody described herein. In some such embodiments, a method further comprises administering to the individual a TH2 pathway inhibitor. In some embodiments, the TH2 pathway inhibitor inhibits at least one target selected from ITK, BTK, IL-9, IL-5, IL-13, IL-4, OX4OL, TSLP, IL-25, IL-33, IgE, IL-9 receptor, IL-5 receptor, IL-4 receptor alpha, IL-13receptoralpha1, IL-13receptoralpha2, OX40, TSLP-R, IL-7Ralpha, IL17RB, ST2, CCR3, CCR4, CRTH2, FcepsilonR1, FcepsilonRII/CD23, Flap, Syk kinase; CCR4, TLR9, CCR3, IL5, IL3, and GM-CSF. In some embodiments, the individual is suffering from moderate to severe asthma. In some embodiments, the individual is suffering from idiopathic pulmonary fibrosis.

In any of the embodiments described herein, the eosinophilic disorder may be selected from asthma, severe asthma, chronic asthma, atopic asthma, atopic dermatitis, allergy, allergic rhinitis, non-allergic rhinitis, contact dermatitis, erythema multiform, bullous skin disease, psoriasis, eczema, rheumatoid arthritis, juvenile chronic arthritis, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angioedema, helminth infections, urticaria, onchocercal dermatitis, eosinophil-associated gastrointestinal disorders, eosinophilic esophagitis, eosinophilic gastritis, eosinophilic gastroenteritis, eosinophilic enteritis, eosinophilic colitis, ulcerative colitis, Whipple's disease, nasal micropolyposis, nasal polyposis, aspirin intolerance, obstructive sleep apnea, Crohn's disease, scleroderma, endomyocardial fibrosis, fibrosis, inflammatory bowel disease, idiopathic interstitial pneumonia, eosinophilic pneumonia, hypersensitivity pneumonitis, goblet cell metaplasia, pulmonary fibrosis, idiopathic pulmonary fibrosis (IPF), pulmonary fibrosis secondary to sclerosis, chronic obstructive pulmonary disease (COPD), hepatic fibrosis, uveitis, cancer, glioblastoma, Hodgkin's lymphoma, and non-Hodgkin's lymphoma. In some embodiments, the IL-13 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma. In any of the embodiments described herein, the IL-4 mediated disease may be selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma. In any of the embodiments described herein, the respiratory disorder may be selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis.

### BRIEF DESCRIPTION OF THE DRAWINGS

FIGS. 1A-1B show that antibody 19C11 is a potent antagonist of IL-4 receptor activation, as described in Example 2. (FIG. 1A) 19C11 blocks IL-4 binding to immobilized IL-4Rα. 19C11 (filled circle), control IgG (open square), no IgG (open triangle). (FIG. 1B) 19C11 antibody inhibits IL-4-induced proliferation of TF-1 cells. 19C11 (filled circle), control IgG (open square), no IgG (open triangle), no IL-4 added (filled triangle).

FIGS. 2A-2B show a Western blot of (FIG. 2A) non-reduced and (FIG. 2B) reduced samples of anti-IL-13.knob and anti-IL-4.hole as IgG1-isotype in E. coli, as described in Example 4. Fragment designations are heavy chain (H) and light chain (L) and lane labels are M (molecular weight standard) C (control, no antibody expression plasmid).

FIGS. 2C-2D show an immunoblot comparing the different isotypes and mutations for anti-IL-13.knob (FIG. 2C) and anti-IL-4.hole (FIG. 2D), as described in Example 5. The upper panels show non-reduced conditions, representing the assembled half-antibody (HL), while the lower panels show reducing conditions, demonstrating that similar amounts of heavy and light chain are synthesized for all variants.

FIGS. 3A-C show analytical characterization of the bispecific antibody, as described in Example 6. (FIG. 3A) Size-exclusion chromatography of the assembled bispecific antibody. The insert shows a zoomed in view of the same graph on the high-molecular weight area. (FIG. 3B) Non-reduced CE-SDS PAGE of the assembled bispecific antibody confirmed formation of the hinge-disulfides and the integrity of inter-chain disulfides. The main peak area corresponds to an intact antibody with formed interchain disulfides. The few minor peak species are reflective of intact antibody lacking complete interchain disulfide to stabilize the hetero-dimer. (FIG. 3C) Reduced CE-SDS confirmed the presence of the expected distribution of light and heavy chains and demonstrated the purity of the material. Aside the main peaks for intact light and heavy chain only trace peaks are detected.

FIGS. 4A-4C show ESI-TOF mass spectrometry analysis of the intact (FIG. 4A) IgG1-, (FIG. 4B) IgG4- and (FIG. 4C) IgG4_{R409K}-isotype based bispecific antibodies, as described in Example 6.

FIGS. 5A-5C show dose-depending inhibition of human IL-4-(FIG. 5A), human IL-13-(FIG. 5B), or human IL-4/IL-13-(Fig. C) induced proliferation by anti-IL-4/IL-13 IgG1-isotype and anti-IL-4/IL-13 IgG4-isotype bispecific antibodies, as described in Example 8. Anti-IL-4/IL-13 IgG1-isotype (filled circles), anti-IL-4/IL-13 IgG4-isotype (open triangles), no antibody added (open square), no cytokine and antibody added (filled square).

FIGS. 6A-6B show dose-depending inhibition of cynomolgus monkey IL-4-(FIG. 6A) and cynomolgus monkey IL-13-(FIG. 6B) induced proliferation by anti-IL-4/IL-13 IgG1-isotype and anti-IL-4/IL-13 IgG4-isotype bispecific antibodies, as described in Example 8. Anti-IL-4/IL-13 IgG1-isotype (filled circles), anti-IL-4/IL-13 IgG4-isotype (filled circles in (FIG. 6A), open triangles in (FIG. 6B)), no antibody added (open square), no cytokine and antibody added (filled square).

FIGS. 7A-B show mean (±SD) serum anti-IL-4/IL-13 IgG4 (FIG. 7A) and IgG1 (FIG. 7B) bispecific antibody concentrations following administration of a single intravenous or subcutaneous dose in cynomolgus monkeys, as described in Example 9. The limit of quantitation (LOQ) for the ELISA was 0.078 μg/mL. All data above LOQ were used and all data below LOQ were excluded. SD was not calculated when n≦2.

FIG. 8 shows bronchoalveolar lavage (BAL) fluid concentrations and epithelial lining fluid (ELF) concentrations of anti-IL-4/IL-13 IgG4 and anti-IL-4/IL-13 IgG1 antibodies following intravenous administration to cynomolgus monkeys challenged with A. suum extract to elicit allergic inflammatory responses that mimic those of asthmatics exposed to allergens, as described in Example 10. The limit of quantitation (LOQ) for the ELISA for anti-IL-4/IL-13 was 0.078 μg/mL. All data above LOQ were used and all data below LOQ were excluded. SD was not calculated when n≦2.

FIGS. 9A-9E show (FIG. 9A) the study design for treatment of an allergic airway inflammation and asthma mouse model, as described in Example 11. FIG. 9 also shows (FIG. 9B) lung eosinophil numbers, (FIG. 9C) bronchoalveolar lavage eosinophil numbers, (FIG. 9D) levels of antigen-specific IgE, and (FIG. 9E) serum TARC levels, in the allergic airway inflammation and asthma mouse model animals following various treatments, as described in Example 11. For each bar graph, the first four bars are, from left to right: control treatment, anti-IL-4 antibody treatment, anti-IL-13 antibody treatment, and anti-IL-4/IL-13 bispecific antibody treatment. The fifth and sixth bars, where present, are naïve mice.

FIG. 10 shows the amino acid sequences for the human κ1 light chain variable region consensus sequence (SEQ ID NO: 61), the mu19C11 antibody light chain variable region (SEQ ID NO: 2), and the 19C11-κ1 graft light chain variable region (SEQ ID NO: 10), as described in Example 3. Positions are numbered according to Kabat and hypervariable regions grafted from mu19C11 to the variable light Kappa I consensus framework are boxed.

FIG. 11 shows the amino acid sequences for the human κ3 light chain variable region consensus sequence (SEQ ID NO: 62), the mu19C11 antibody light chain variable region (SEQ ID NO: 2), and the 19C11-κ3 graft light chain variable region (SEQ ID NO: 11), as described in Example 3. Positions are numbered according to Kabat and hypervariable regions grafted from mu19C11 to the variable light Kappa I consensus framework are boxed.

FIG. 12 shows the amino acid sequences for the human VH1 heavy chain variable region consensus sequence (SEQ ID NO: 63), the mu19C11 antibody heavy chain variable region (SEQ ID NO: 1), and the 19C11-VH1 graft (SEQ ID NO: 3), the 19C11-VH1.L (SEQ ID NO: 4), and 19C11-VH1.FFL (SEQ ID NO: 5) heavy chain variable regions, as described in Example 3. Positions are numbered according to Kabat and hypervariable regions and vernier positions taken from mu19C11 to the variable heavy subgroup I consensus framework are boxed.

FIG. 13 shows the amino acid sequences for the human VH3 heavy chain variable region consensus sequence (SEQ ID NO: 64), the mu19C11 antibody heavy chain variable region (SEQ ID NO: 1), and the 19C11-VH3 graft (SEQ ID NO: 6), the 19C11-VH3.FLA (SEQ ID NO: 7), 19C11-VH3.LA (SEQ ID NO: 8), and 19C11-VH3.LA.SV (SEQ ID NO: 9) heavy chain variable regions, as described in Example 3. Positions are numbered according to Kabat and hypervariable regions and vernier positions taken from mu19C11 to the variable heavy subgroup I consensus framework are boxed.

FIG. 14 shows a table of surface plasmon resonance (SPR) affinity measurements of the humanized antibodies for IL-4, as described in Example 3.

FIG. 15 shows a plot of inhibition of biotinylated human IL-4 binding to human IL-4R by increasing concentrations of anti-IL-4/IL-13 bispecific antibody, as described in Example 7.

FIG. 16 shows a plot of inhibition of biotinylated human IL-13 binding to human IL-13Rα1 by increasing concentrations of anti-IL-4/IL-13 bispecific antibody, as described in Example 7.

FIG. 17 shows a plot of inhibition of biotinylated human IL-13 binding to human IL-13Rα2 by increasing concentrations of anti-IL-4/IL-13 bispecific antibody, as described in Example 7.

FIG. 18 shows SPR sensograms for binding of IL-13Rα2 to IL-13 in the presence of anti-IL-4/IL-13 bispecific antibody, as described in Example 7. The lines shown represent a two-fold concentration series of the receptor ranging from 12.5 nM to 200 nM.

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### CERTAIN DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used in this specification and the appended claims, the singular forms “a,” “an” and “the” include plural referents unless the context clearly dictates otherwise. Thus, for example, reference to “a protein” or an “antibody” includes a plurality of proteins or antibodies, respectively; reference to “a cell” includes mixtures of cells, and the like.

The term “biological sample” as used herein includes, but is not limited to, blood, serum, plasma, sputum, bronchoalveolar lavage, tissue biopsies (e.g., lung samples), and nasal samples including nasal swabs or nasal polyps.

FE_{NO} assay refers to an assay that measures FE_{NO} (fractional exhaled nitric oxide) levels. Such levels can be evaluated using, e.g., a hand-held portable device, NIOX MINO™ (Aerocrine, Solna, Sweden), in accordance with guidelines published by the American Thoracic Society (ATS) in 2005. FE_{NO} may be noted in other similar ways, e.g., FeNO or FENO, and it should be understood that all such similar variations have the same meaning.

Asthma is a complex disorder characterized by variable and recurring symptoms, reversible airflow obstruction (e.g., by bronchodilator) and bronchial hyperresponsiveness which may or may not be associated with underlying inflammation. Examples of asthma include aspirin sensitive/exacerbated asthma, atopic asthma, severe asthma, mild asthma, moderate to severe asthma, corticosteroid naïve asthma, chronic asthma, corticosteroid resistant asthma, corticosteroid refractory asthma, newly diagnosed and untreated asthma, asthma due to smoking, asthma uncontrolled on corticosteroids and other asthmas as mentioned in J Allergy Clin Immunol (2010) 126(5):926-938.

“Eosinophilic Disorder” means a disorder associated with excess eosinophil numbers in which atypical symptoms may manifest due to the levels or activity of eosinophils locally or systemically in the body. Disorders associated with excess eosinophil numbers or activity include, but are not limited to, asthma (including aspirin sensitive asthma, chronic asthma, and severe asthma), atopic asthma, atopic dermatitis, allergy, allergic rhinitis (including seasonal allergic rhinitis), non-allergic rhinitis, contact dermatitis, erythema multiform, bullous skin diseases, psoriasis, eczema, rheumatoid arthritis, juvenile chronic arthritis, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angiodema, helminth infections, urticaria, onchocercal dermatitis, Eosinophil-Associated Gastrointestinal Disorders (EGID) (including but not limited to, eosinophilic esophagitis, eosinophilic gastritis, eosinophilic gastroenteritis, eosinophilic enteritis, and eosinophilic colitis), ulcerative colitis, Whipple's disease, nasal micropolyposis and polyposis, aspirin intolerance, obstructive sleep apnea, Crohn's disease, scleroderma, endomyocardial fibrosis, cancer (e.g., glioblastoma (such as glioblastoma multiforme), non-Hodgkin's lymphoma (NHL), Hodgkin's lymphoma), fibrosis, inflammatory bowel disease, idiopathic interstitial pneumonia, eosinophilic pneumonia, hypersensitivity pneumonitis, goblet cell metaplasia, pulmonary fibrosis (including idiopathic pulmonary fibrosis (IPF) and pulmonary fibrosis secondary to sclerosis), chronic obstructive pulmonary disease (COPD), hepatic fibrosis, and uveitis. Eosinophil-derived secretory products have also been associated with the promotion of angiogenesis and connective tissue formation in tumors and the fibrotic responses seen in conditions such as chronic asthma, Crohn's disease, scleroderma, and endomyocardial fibrosis (Munitz A, Levi-Schaffer F. Allergy 2004; 59: 268-75, Adamko et al. Allergy 2005; 60: 13-22, Oldhoff, et al. Allergy 2005; 60: 693-6).

IL-13 mediated disorder means a disorder associated with excess IL-13 levels or activity in which atypical symptoms may manifest due to the levels or activity of IL-13 locally and/or systemically in the body. Examples of IL-13 mediated disorders include: cancers (e.g., non-Hodgkin's lymphoma, glioblastoma), atopic dermatitis, allergic rhinitis, asthma, fibrosis, inflammatory bowel disease, Crohn's disease, lung inflammatory disorders (including pulmonary fibrosis such as IPF), COPD, and hepatic fibrosis.

IL-4 mediated disorder means: a disorder associated with excess IL-4 levels or activity in which atypical symptoms may manifest due to the levels or activity of IL-4 locally and/or systemically in the body. Examples of IL-4 mediated disorders include: cancers (e.g., non-Hodgkin's lymphoma, glioblastoma), atopic dermatitis, allergic rhinitis, asthma, fibrosis, inflammatory bowel disease, Crohn's disease, lung inflammatory disorders (including pulmonary fibrosis such as IPF), COPD, and hepatic fibrosis.

Asthma-Like Symptom includes a symptom selected from the group consisting of shortness of breath, cough (changes in sputum production and/or sputum quality and/or cough frequency), wheezing, chest tightness, bronchioconstriction and nocturnal awakenings ascribed to one of the symptoms above or a combination of these symptoms (Juniper et al (2000) Am. J. Respir. Crit. Care Med., 162(4), 1330-1334.).

The term “respiratory disorder” includes, but is not limited to, asthma (e.g., allergic and non-allergic asthma (e.g., due to infection, e.g., with respiratory syncytial virus (RSV), e.g., in younger children)); bronchitis (e.g., chronic bronchitis); chronic obstructive pulmonary disease (COPD) (e.g., emphysema (e.g., cigarette-induced emphysema); conditions involving airway inflammation, eosinophilia, fibrosis and excess mucus production, e.g., cystic fibrosis, pulmonary fibrosis, and allergic rhinitis. Examples of diseases that can be characterized by airway inflammation, excessive airway secretion, and airway obstruction include asthma, chronic bronchitis, bronchiectasis, and cystic fibrosis.

Exacerbations (commonly referred to as asthma attacks or acute asthma) are episodes of new or progressive increase in shortness of breath, cough (changes in sputum production and/or sputum quality and/or cough frequency), wheezing, chest tightness, nocturnal awakenings ascribed to one of the symptoms above or a combination of these symptoms. Exacerbations are often characterized by decreases in expiratory airflow (PEF or FEV1). However, PEF variability does not usually increase during an exacerbation, although it may do so leading up to or during the recovery from an exacerbation. The severity of exacerbations ranges from mild to life-threatening and can be evaluated based on both symptoms and lung function. Severe asthma exacerbations as described herein include exacerbations that result in any one or combination of the following hospitalization for asthma treatment, high corticosteroid use (e.g., quadrupling the total daily corticosteroid dose or a total daily dose of greater or equal to 500 micrograms of FP or equivalent for three consecutive days or more), or oral/parenteral corticosteroid use.

A “TH2 pathway inhibitor” or “TH2 inhibitor” is an agent that inhibits the TH2 pathway. Examples of a TH2 pathway inhibitor include inhibitors of the activity of any one of the targets selected from the group consisting of: ITK, BTK, IL-9 (e.g., MEDI-528), IL-5 (e.g., Mepolizumab, CAS No. 196078-29-2; resilizumab), IL-13 (e.g., IMA-026, IMA-638 (also referred to as, anrukinzumab, INN No. 910649-32-0; QAX-576; IL-4/IL-13 trap), tralokinumab (also referred to as CAT-354, CAS No. 1044515-88-9); AER-001, ABT-308 (also referred to as humanized 13C5.5 antibody), IL-4 (e.g., AER-001, IL-4/IL-13 trap), OX4OL, TSLP, IL-25, IL-33 and IgE (e.g., XOLAIR, QGE-031; MEDI-4212); and receptors such as: IL-9 receptor, IL-5 receptor (e.g., MEDI-563 (benralizumab, CAS No. 1044511-01-4), IL-4receptor alpha (e.g., AMG-317, AIR-645), IL-13receptoralpha1 (e.g., R-1671) and IL-13receptoralpha2, OX40, TSLP-R, IL-7Ralpha (a co-receptor for TSLP), IL17RB (receptor for IL-25), ST2 (receptor for IL-33), CCR3, CCR4, CRTH2 (e.g., AMG-853, AP768, AP-761, MLN6095, ACT129968), FcepsilonR1, FcepsilonRII/CD23 (receptors for IgE), Flap (e.g., GSK2190915), Syk kinase (R-343, PF3526299); CCR4 (AMG-761), TLR9 (QAX-935) and multi-cytokine inhibitor of CCR3, IL5, IL3, GM-CSF (e.g., TPI ASM8). Examples of inhibitors of the aforementioned targets are disclosed in, for example, WO2008/086395; WO2006/085938; U.S. Pat. No. 7,615,213; U.S. Pat. No. 7,501,121; WO2006/085938; WO 2007/080174; U.S. Pat. No. 7,807,788; WO2005007699; WO2007036745; WO2009/009775; WO2007/082068; WO2010/073119; WO2007/045477; WO2008/134724; US2009/0047277; and WO2008/127,271).

The term “small molecule” refers to an organic molecule having a molecular weight between 50 Daltons to 2500 Daltons.

The term “antibody” is used in the broadest sense and specifically covers, for example, monoclonal antibodies, polyclonal antibodies, antibodies with polyepitopic specificity, single chain antibodies, multi-specific antibodies and fragments of antibodies. Such antibodies can be chimeric, humanized, human and synthetic. Such antibodies and methods of generating them are described in more detail below.

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). In some embodiments, an antigen-binding domain of a multispecific antibody (such as a bispecific antibody) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, antibody fragments such as Fab, Fv, dsFv, scFv, diabodies, bispecific diabodies and triabodies, antibody fragments that have been linked covalently or non-covalently. A VH/VL unit that further comprises at least a portion of a heavy chain constant region and/or at least a portion of a light chain constant region may also be referred to as a “hemimer” or “half antibody.” According to some embodiments, the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM. In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.”

The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, C_{L}:C_{H1} interfaces or V_{H}/V_{L} interfaces of antibodies (see, e.g., US 2011/0287009, US2007/0178552, WO 96/027011, WO 98/050431, and Zhu et al., 1997, Protein Science 6:781-788). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

The term “therapeutic agent” refers to any agent that is used to treat a disease. A therapeutic agent may be, for example, a polypeptide(s) (e.g., an antibody, an immunoadhesin or a peptibody), an aptamer or a small molecule that can bind to a protein or a nucleic acid molecule that can bind to a nucleic acid molecule encoding a target (i.e., siRNA), etc.

The term “controller” or “preventor” refers to any therapeutic agent that is used to control asthma inflammation. Examples of controllers include corticosteroids, leukotriene receptor antagonists (e.g., inhibit the synthesis or activity of leukotrienes such as montelukast, zileuton, pranlukast, zafirlukast), LABAs, corticosteroid/LABA combination compositions, theophylline (including aminophylline), cromolyn sodium, nedocromil sodium, omalizumab, LAMAs, MABA (e.g, bifunctional muscarinic antagonist-beta2 Agonist), 5-Lipoxygenase Activating Protein (FLAP) inhibitors, and enzyme PDE-4 inhibitor (e.g., roflumilast). A “second controller” typically refers to a controller that is not the same as the first controller.

The term “corticosteroid sparing” or “CS” means the decrease in frequency and/or amount, or the elimination of, corticosteroid used to treat a disease in a patient taking corticosteroids for the treatment of the disease due to the administration of another therapeutic agent. A “CS agent” refers to a therapeutic agent that can cause CS in a patient taking a corticosteroid.

The term “corticosteroid” includes, but is not limited to fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future. Examples of corticosteroids that can be inhaled and are combined with a long-acting beta2-agonist include, but are not limited to:

budesonide/formoterol and fluticasone/salmeterol.

Examples of corticosteroid/LABA combination drugs include fluticasone furoate/vilanterol trifenatate and indacaterol/mometasone.

The term “LABA” means long-acting beta-2 agonist, which agonist includes, for example, salmeterol, formoterol, bambuterol, albuterol, indacaterol, arformoterol and clenbuterol.

The term “LAMA” means long-acting muscarinic antagonist, which agonists include: tiotropium.

Examples of LABA/LAMA combinations include, but are not limited to: olodaterol tiotropium (Boehringer Ingelheim's) and indacaterol glycopyrronium (Novartis)

The term “SABA” means short-acting beta-2 agonists, which agonists include, but are not limited to, salbutamol, levosalbutamol, fenoterol, terbutaline, pirbuterol, procaterol, bitolterol, rimiterol, carbuterol, tulobuterol and reproterol

Leukotriene receptor antagonists (sometimes referred to as a leukast) (LTRA) are drugs that inhibit leukotrienes. Examples of leukotriene inhibitors include montelukast, zileuton, pranlukast, and zafirlukast.

The term “FEV1” refers to the volume of air exhaled in the first second of a forced expiration. It is a measure of airway obstruction. Provocative concentration of methacholine required to induce a 20% decline in FEV1 (PC20) is a measure of airway hyperresponsiveness. FEV1 may be noted in other similar ways, e.g., FEV₁, and it should be understood that all such similar variations have the same meaning.

The term “relative change in FEV1”=(FEV1 at week 12 of treatment−FEV1 prior to start of treatment) divided by FEV1.

As used herein, “FVC” refers to “Forced Vital Capacity” which refers to a standard test that measures the change in lung air volume between a full inspiration and maximal expiration to residual volume (as opposed to the volume of air expelled in one second as in FEV1). It is a measure of the functional lung capacity. In patients with restrictive lung diseases such as interstitial lung disease including IPF, hypersensitivity pneumonitis, sarcoidosis, and systemic sclerosis, the FVC is reduced typically due to scarring of the lung parenchyma.

The term “mild asthma” refers to a patient generally experiencing symptoms or exacerbations less than two times a week, nocturnal symptoms less than two times a month, and is asymptomatic between exacerbations. Mild, intermittent asthma is often treated as needed with the following: inhaled bronchodilators (short-acting inhaled beta2-agonists); avoidance of known triggers; annual influenza vaccination; pneumococcal vaccination every 6 to 10 years, and in some cases, an inhaled beta2-agonist, cromolyn, or nedocromil prior to exposure to identified triggers. If the patient has an increasing need for short-acting beta2-agonist (e.g., uses short-acting beta2-agonist more than three to four times in 1 day for an acute exacerbation or uses more than one canister a month for symptoms), the patient may require a stepup in therapy.

The term “moderate asthma” generally refers to asthma in which the patient experiences exacerbations more than two times a week and the exacerbations affect sleep and activity; the patient has nighttime awakenings due to asthma more than two times a month; the patient has chronic asthma symptoms that require short-acting inhaled beta2-agonist daily or every other day; and the patient's pretreatment baseline PEF or FEV1 is 60 to 80 percent predicted and PEF variability is 20 to 30 percent.

The term “severe asthma” generally refers to asthma in which the patient has almost continuous symptoms, frequent exacerbations, frequent nighttime awakenings due to the asthma, limited activities, PEF or FEV1 baseline less than 60 percent predicted, and PEF variability of 20 to 30 percent.

Examples of rescue medications include albuterol, ventolin and others.

“Resistant” refers to a disease that demonstrates little or no clinically significant improvement after treatment with a therapeutic agent. For example, asthma which requires treatment with high dose ICS (e.g., quadrupling the total daily corticosteroid dose or a total daily dose of greater or equal to 500 micrograms of FP (or equivalent) for at least three consecutive days or more, or systemic corticosteroid for a two week trial to establish if asthma remains uncontrolled or FEV1 does not improve is often considered severe refractory asthma.

A therapeutic agent as provided herein can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In some embodiments, the therapeutic agent is inhaled. According to some embodiments, the dosing is given by injections, e.g., intravenous or subcutaneous injections. In some embodiments, the therapeutic agent is administered using a syringe (e.g., prefilled or not) or an autoinjector.

For the prevention or treatment of disease, the appropriate dosage of a therapeutic agent may depend on the type of disease to be treated, the severity and course of the disease, whether the therapeutic agent is administered for preventive or therapeutic purposes, previous therapy, the patient's clinical history and response to the therapeutic agent, and the discretion of the attending physician. The therapeutic agent is suitably administered to the patient at one time or over a series of treatments. The therapeutic agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

“Patient response” or “response” (and grammatical variations thereof) can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described herein.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-IL-4 antibody” and “an antibody that binds to IL-4” refer to an antibody that is capable of binding IL-4 with sufficient affinity such that the antibody is useful as a diagnostic and/or therapeutic agent in targeting IL-4. In some embodiments, the extent of binding of an anti-IL-4 antibody to an unrelated, non-IL-4 protein is less than about 10% of the binding of the antibody to IL-4 as measured, e.g., by a radioimmunoassay (RIA). In certain embodiments, an antibody that binds to IL-4 has a dissociation constant (Kd) of ≦1 μM, ≦100 nM, ≦10 nM, ≦1 nM, ≦0.1 nM, ≦0.01 nM, or ≦0.001 nM (e.g. 10-8 M or less, e.g. from 10-8 M to 10-13 M, e.g., from 10-9 M to 10-13 M). In certain embodiments, an anti-IL-4 antibody binds to an epitope of IL-4 that is conserved among IL-4 from different species. In some embodiments, an anti-IL-4 antibody is a multispecific antibody, such as a bispecific antibody.

The terms “anti-IL-13 antibody” and “an antibody that binds to IL-13” refer to an antibody that is capable of binding IL-13 with sufficient affinity such that the antibody is useful as a diagnostic and/or therapeutic agent in targeting IL-13. In some embodiments, the extent of binding of an anti-IL-13 antibody to an unrelated, non-IL-13 protein is less than about 10% of the binding of the antibody to IL-13 as measured, e.g., by a radioimmunoassay (RIA). In certain embodiments, an antibody that binds to IL-13 has a dissociation constant (Kd) of ≦1 μM, ≦100 nM, ≦10 nM, ≦1 nM, ≦0.1 nM, ≦0.01 nM, or ≦0.001 nM (e.g. 10-8 M or less, e.g. from 10-8 M to 10-13 M, e.g., from 10-9 M to 10-13 M). In certain embodiments, an anti-IL-13 antibody binds to an epitope of IL-13 that is conserved among IL-13 from different species. In some embodiments, an anti-IL-13 antibody is a multispecific antibody, such as a bispecific antibody.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In some embodiments, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In some embodiments, for the VL, the subgroup is subgroup kappa I as in Kabat et al., supra. In some embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

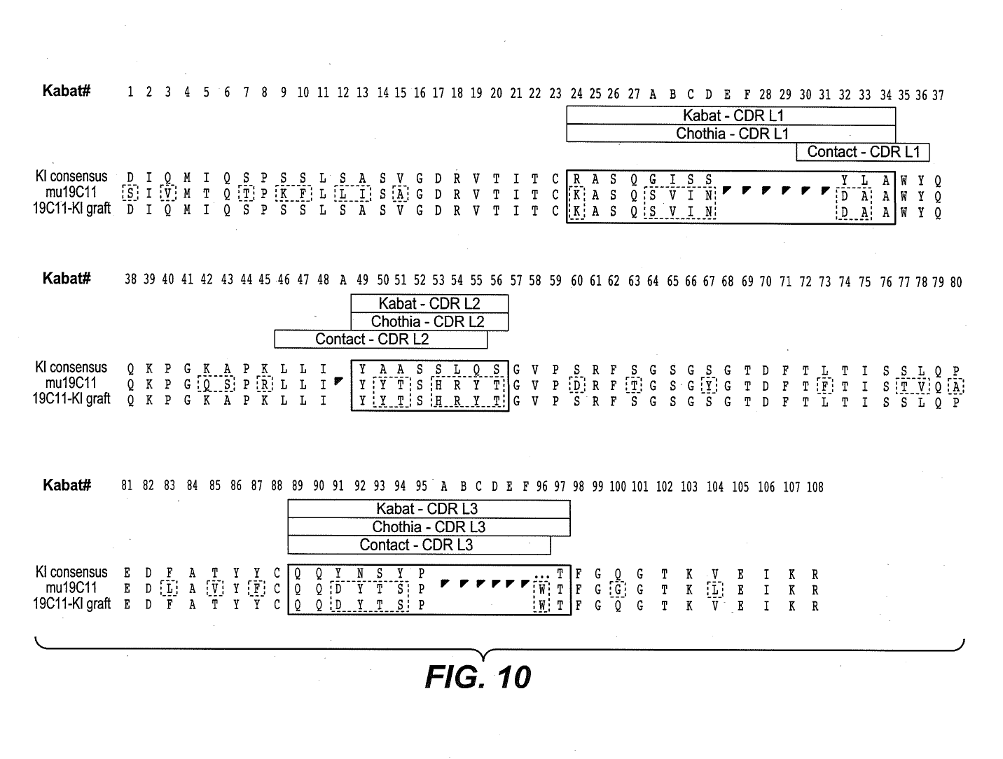

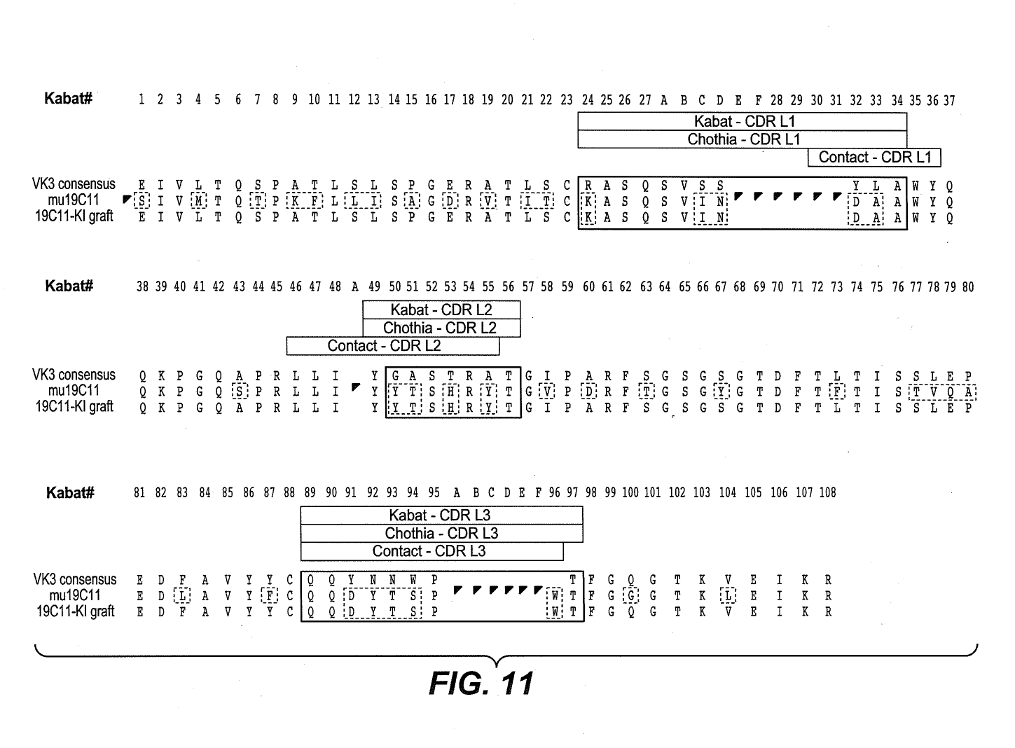

In some embodiments, HVR residues comprise those identified in FIGS. 10 to 13 or elsewhere in the specification.

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-IL-4 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

“Isolated nucleic acid encoding an anti-IL3 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, monoclonal antibodies may be made by a variety of techniques, including but not limited to the hybridoma method, recombinant DNA methods, phage-di splay methods, and methods utilizing transgenic animals containing all or part of the human immunoglobulin loci, such methods and other exemplary methods for making monoclonal antibodies being described herein. In some embodiments, a monoclonal antibody is a multispecific (such as bispecific) antibody.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products. The term “package insert” is also used to refer to instructions customarily included in commercial packages of diagnostic products that contain information about the intended use, test principle, preparation and handling of reagents, specimen collection and preparation, calibration of the assay and the assay procedure, performance and precision data such as sensitivity and specificity of the assay.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “IL-4,” as used herein, refers to any native IL-4 from any vertebrate source, including mammals such as primates (e.g. humans) and rodents (e.g., mice and rats), unless otherwise indicated. The term encompasses “full-length,” unprocessed IL-4 as well as any form of IL-4 that results from processing in the cell. The term also encompasses naturally occurring variants of IL-4, e.g., splice variants or allelic variants. The amino acid sequences of exemplary human IL-4 are shown in SEQ ID NOs: 27 and 28, and in Swiss-Prot Accession No. P05112.2. The amino acid sequence of an exemplary cynomolgus monkey IL-4 is shown in SEQ ID NO: 33.

The term “IL-13,” as used herein, refers to any native IL-13 from any vertebrate source, including mammals such as primates (e.g. humans) and rodents (e.g., mice and rats), unless otherwise indicated. The term encompasses “full-length,” unprocessed IL-13 as well as any form of IL-13 that results from processing in the cell. The term also encompasses naturally occurring variants of IL-13, e.g., splice variants or allelic variants. The amino acid sequences of exemplary human IL-13 are shown in SEQ ID NOs: 29 and 30, and in Swiss-Prot Accession No. P35225.2. The amino acid sequence of an exemplary cynomolgus monkey IL-13 is shown in SEQ ID NO: 32.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### Compositions and Methods

In certain embodiments, antibodies that bind to IL-4 are provided. In certain embodiments, bispecific antibodies that bind to IL-4 and IL-13 are provided. The antibodies are useful, e.g., for the diagnosis or treatment of eosinophilic disorders, including respiratory disorders (such as asthma and IPF), IL-4 mediated disorders, and IL-13 mediated disorders.

### Exemplary Anti-IL-4 Antibodies

In some embodiments, isolated antibodies that bind IL-4 are provided. In some embodiments, an anti-IL-4 antibody comprises at least one, two, three, four, five, or six HVRs selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising the amino acid sequence of SEQ ID NO: 17.

In some embodiments, an antibody is provided that comprises at least one, at least two, or all three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; and (c) HVR-H3 comprising the amino acid sequence of SEQ ID NO: 14. In some embodiments, the antibody comprises HVR-H3 comprising the amino acid sequence of SEQ ID NO: 14. In some embodiments, the antibody comprises HVR-H3 comprising the amino acid sequence of SEQ ID NO: 14 and HVR-L3 comprising the amino acid sequence of SEQ ID NO: 17. In some embodiments, the antibody comprises HVR-H3 comprising the amino acid sequence of SEQ ID NO: 14, HVR-L3 comprising the amino acid sequence of SEQ ID NO: 17, and HVR-H2 comprising the amino acid sequence of SEQ ID NO: 13 or SEQ ID NO: 18. In some embodiments, the antibody comprises (a) HVR-H1 comprising the amino acid sequence of SEQ ID NO: 12; (b) HVR-H2 comprising the amino acid sequence of SEQ ID NO: 13 or SEQ ID NO: 18; and (c) HVR-H3 comprising the amino acid sequence of SEQ ID NO: 14.

In some embodiments, an antibody is provided that comprises at least one, at least two, or all three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17. In some embodiments, the antibody comprises (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17.

In some embodiments, an antibody comprises (a) a VH domain comprising at least one, at least two, or all three VH HVR sequences selected from (i) HVR-H1 comprising the amino acid sequence of SEQ ID NO: 12, (ii) HVR-H2 comprising the amino acid sequence of SEQ ID NO: 13 or SEQ ID NO: 18, and (iii) HVR-H3 comprising an amino acid sequence selected from SEQ ID NO: 14; and (b) a VL domain comprising at least one, at least two, or all three VL HVR sequences selected from (i) HVR-L1 comprising the amino acid sequence of SEQ ID NO: 15, (ii) HVR-L2 comprising the amino acid sequence of SEQ ID NO: 16, and (c) HVR-L3 comprising the amino acid sequence of SEQ ID NO: 17.

In some embodiments, an antibody is provided that comprises (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising an amino acid sequence selected from SEQ ID NO: 17. In some embodiments, an antibody is provided that comprises (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising an amino acid sequence selected from SEQ ID NO: 17.

In any of the above embodiments, an anti-IL-4 antibody is humanized. In some embodiments, an anti-IL-4 antibody comprises HVRs as in any of the above embodiments, and further comprises an acceptor human framework, e.g. a human immunoglobulin framework or a human consensus framework. In some embodiments, an anti-IL-4 antibody comprises HVRs as in any of the above embodiments, and further comprises a VH comprising FR1, FR2, FR3, and FR4 of any one of SEQ ID NOs: 3 to 9. In some embodiments, an anti-IL-4 antibody comprises HVRs as in any of the above embodiments, and further comprises a VH comprising FR1, FR2, FR3, and FR4 of SEQ ID NO: 9. In some embodiments, an anti-IL-4 antibody comprises HVRs as in any of the above embodiments, and further comprises a VL comprising FR1, FR2, FR3, and FR4 of any one of SEQ ID NOs: 10 and 11. In some embodiments, an anti-IL-4 antibody comprises HVRs as in any of the above embodiments, and further comprises a VL comprising FR1, FR2, FR3, and FR4 of SEQ ID NO: 10.

In some embodiments, an anti-IL-4 antibody comprises a heavy chain variable domain (VH) sequence having at least 90%, 91%, 92%, 93%, 94%, 95%, 96%, 97%, 98%, 99%, or 100% sequence identity to the amino acid sequence of any one of SEQ ID NOs: 1 and 3 to 9. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-4 antibody comprising that sequence retains the ability to bind to IL-4. In certain embodiments, a total of 1 to 10 amino acids have been substituted, inserted and/or deleted in SEQ ID NO: 9. In certain embodiments, substitutions, insertions, or deletions occur in regions outside the HVRs (i.e., in the FRs). Optionally, the anti-IL-4 antibody comprises the VH sequence in SEQ ID NO: 9, including post-translational modifications of that sequence. In a particular embodiment, the VH comprises one, two or three HVRs selected from: (a) HVR-H1 comprising the amino acid sequence of SEQ ID NO: 12, (b) HVR-H2 comprising the amino acid sequence of SEQ ID NO: 13 or SEQ ID NO: 18, and (c) HVR-H3 comprising the amino acid sequence of SEQ ID NO: 14.

In some embodiments, an anti-IL-4 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of any one of SEQ ID NOs: 2, 10, and 11. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-4 antibody comprising that sequence retains the ability to bind to IL-4. In certain embodiments, a total of 1 to 10 amino acids have been substituted, inserted and/or deleted in SEQ ID NO: 10. In certain embodiments, the substitutions, insertions, or deletions occur in regions outside the HVRs (i.e., in the FRs). Optionally, the anti-IL-4 antibody comprises the VL sequence in SEQ ID NO: 10, including post-translational modifications of that sequence. In a particular embodiment, the VL comprises one, two or three HVRs selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17.

In some embodiments, an anti-IL-4 antibody is provided, wherein the antibody comprises a VH as in any of the embodiments provided above, and a VL as in any of the embodiments provided above. In some embodiments, the antibody comprises the VH and VL sequences in SEQ ID NO: 9 and SEQ ID NO: 10, respectively, including post-translational modifications of those sequences.

In some embodiments, an antibody is provided that competes for binding to IL-4 with an anti-IL-4 antibody comprising a VH sequence of SEQ ID NO: 9 and a VL sequence of SEQ ID NO: 10. In some embodiments, an antibody is provided that binds to the same epitope as an anti-IL-4 antibody provided herein. For example, in certain embodiments, an antibody is provided that binds to the same epitope as an anti-IL-4 antibody comprising a VH sequence of SEQ ID NO: 9 and a VL sequence of SEQ ID NO: 10.

In some embodiments, an anti-IL-4 antibody according to any of the above embodiments is a monoclonal antibody, including a chimeric, humanized or human antibody. In some embodiments, an anti-IL-4 antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-4 antibody according to any of the above embodiments may incorporate any of the features, singly or in combination, as described in Sections 1-7 below.

### Exemplary Anti-IL-13 Antibodies

In some embodiments, isolated antibodies that bind IL-13 are provided. In some embodiments, an anti-IL-13 antibody comprises at least one, two, three, four, five, or six HVRs selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, an antibody is provided that comprises at least one, at least two, or all three VH HVR sequences selected from (a) HVR-H1 comprising the amino acid sequence of SEQ ID NO: 21 or SEQ ID NO: 60; (b) HVR-H2 comprising the amino acid sequence of SEQ ID NO: 22; and (c) HVR-H3 comprising the amino acid sequence of SEQ ID NO: 23. In some embodiments, the antibody comprises HVR-H3 comprising the amino acid sequence of SEQ ID NO: 23. In some embodiments, the antibody comprises HVR-H3 comprising the amino acid sequence of SEQ ID NO: 23 and HVR-L3 comprising the amino acid sequence of SEQ ID NO: 26. In some embodiments, the antibody comprises HVR-H3 comprising the amino acid sequence of SEQ ID NO: 23, HVR-L3 comprising the amino acid sequence of SEQ ID NO: 26, and HVR-H2 comprising the amino acid sequence of SEQ ID NO: 22. In some embodiments, the antibody comprises (a) HVR-H1 comprising the amino acid sequence of SEQ ID NO: 21 or SEQ ID NO: 60; (b) HVR-H2 comprising the amino acid sequence of SEQ ID NO: 22; and (c) HVR-H3 comprising the amino acid sequence of SEQ ID NO: 23.

In some embodiments, an antibody is provided that comprises at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, the antibody comprises (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, an antibody comprises (a) a VH domain comprising at least one, at least two, or all three VH HVR sequences selected from (i) HVR-H1 comprising the amino acid sequence of SEQ ID NO: 21 or SEQ ID NO: 60, (ii) HVR-H2 comprising the amino acid sequence of SEQ ID NO: 22, and (iii) HVR-H3 comprising an amino acid sequence selected from SEQ ID NO: 23; and (b) a VL domain comprising at least one, at least two, or all three VL HVR sequences selected from (i) HVR-L1 comprising the amino acid sequence of SEQ ID NO: 24, (ii) HVR-L2 comprising the amino acid sequence of SEQ ID NO: 25, and (c) HVR-L3 comprising the amino acid sequence of SEQ ID NO: 26.

In some embodiments, an antibody is provided that comprises (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising an amino acid sequence selected from SEQ ID NO: 26.

In any of the above embodiments, an anti-IL-13 antibody is humanized. In some embodiments, an anti-IL-13 antibody comprises HVRs as in any of the above embodiments, and further comprises an acceptor human framework, e.g. a human immunoglobulin framework or a human consensus framework. In some embodiments, an anti-IL-13antibody comprises HVRs as in any of the above embodiments, and further comprises a VH comprising FR1, FR2, FR3, and/or FR4 sequences of SEQ ID NO: 19. In some embodiments, an anti-IL-13antibody comprises HVRs as in any of the above embodiments, and further comprises a VL comprising FR1, FR2, FR3, and/or FR4 sequences of SEQ ID NO: 20. In some embodiments, an anti-IL-13antibody comprises HVRs as in any of the above embodiments, and further comprises a VH comprising FR1, FR2, FR3, and/or FR4 sequences of SEQ ID NO: 56. In some embodiments, an anti-IL-13antibody comprises HVRs as in any of the above embodiments, and further comprises a VL comprising FR1, FR2, FR3, and/or FR4 sequences of SEQ ID NO: 57.

In some embodiments, an anti-IL-13 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 19. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 19. In certain embodiments, substitutions, insertions, or deletions occur in regions outside the HVRs (i.e., in the FRs). In some embodiments, the anti-IL-13 antibody comprises the VH sequence in SEQ ID NO: 19, including post-translational modifications of that sequence. In some embodiments, the anti-IL-13 antibody comprises the VH sequence in SEQ ID NO: 56, including post-translational modifications of that sequence. In some embodiments, the VH comprises one, two or three HVRs selected from: (a) HVR-H1 comprising the amino acid sequence of SEQ ID NO: 21 or SEQ ID NO: 60, (b) HVR-H2 comprising the amino acid sequence of SEQ ID NO: 22, and (c) HVR-H3 comprising the amino acid sequence of SEQ ID NO: 23.

In some embodiments, an anti-IL-13 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 20. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 20. In certain embodiments, the substitutions, insertions, or deletions occur in regions outside the HVRs (i.e., in the FRs). In some embodiments, the anti-IL-13 antibody comprises the VL sequence in SEQ ID NO: 20, including post-translational modifications of that sequence. In some embodiments, the anti-IL-13 antibody comprises the VL sequence in SEQ ID NO: 57, including post-translational modifications of that sequence. In some embodiments, the VL comprises one, two or three HVRs selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, an anti-IL-13 antibody is provided, wherein the antibody comprises a VH as in any of the embodiments provided above, and a VL as in any of the embodiments provided above. In some embodiments, the antibody comprises the VH sequence in SEQ ID NO: 19 or SEQ ID NO: 56 and the VL sequence in SEQ ID NO: 20 or SEQ ID NO: 57, including post-translational modifications of those sequences.

In some embodiments, an antibody is provided that competes for binding to IL-13 with an anti-IL-13 antibody comprising a VH sequence of SEQ ID NO: 19 and a VL sequence of SEQ ID NO: 20. In some embodiments, an antibody is provided that binds to the same epitope as an anti-IL-13 antibody provided herein. See, e.g., Ultsch, M. et al., Structural Basis of Signaling Blockade by Anti-IL-13 Antibody Lebrikizumab, J. Mol. Biol. (2013), dx.doi.org/10.1016/j.jmb.2013.01.024. In some embodiments, an antibody is provided that binds to the same epitope as an anti-IL-13 antibody provided herein. For example, in certain embodiments, an antibody is provided that binds to the same epitope as an anti-IL-13 antibody comprising a VH sequence of SEQ ID NO: 19 and a VL sequence of SEQ ID NO: 20. In certain embodiments, an antibody is provided that binds to an epitope within amino acids 63 to 74 of human precursor IL-13 (SEQ ID NO: 29) or amino acids 45 to 56 of the mature form of human IL-13 (SEQ ID NO: 30), which are YCAALESLINVS (SEQ ID NO: 43). In certain embodiments, an antibody is provided that binds to an epitope within amino acids 68 to 75 of human precursor IL-13 (SEQ ID NO: 29) or amino acids 50-57 of the mature form of human IL-13 (SEQ ID NO: 30), which are ESLINVSG (SEQ ID NO: 42).

Another exemplary anti-IL-13 antibody is 11H4 and humanized versions thereof, including hu11H4v6. Mu11H4 comprises heavy chain and light chain variable regions comprising the amino acid sequences of SEQ ID NOs: 45 and 44, respectively. Humanized hu11H4v6 comprises a heavy chain variable region and a light chain variable region comprising the amino acid sequence of SEQ ID NOs: 49 and 48, respectively. Humanized hu11H4v6 comprises a heavy chain and a light chain comprising the amino acid sequence of SEQ ID NOs: 47 and 46, respectively.

In some embodiments, an anti-IL-13 antibody comprises at least one, two, three, four, five, or six HVRs selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; (d) HVR-L1 comprising the amino acid sequence of SEQ ID NO: 53; (e) HVR-L2 comprising the amino acid sequence of SEQ ID NO: 54; and (f) HVR-L3 comprising the amino acid sequence of SEQ ID NO: 55.

In some embodiments, an antibody is provided that comprises at least one, at least two, or all three VH HVR sequences selected from (a) HVR-H1 comprising the amino acid sequence of SEQ ID NO: 50; (b) HVR-H2 comprising the amino acid sequence of SEQ ID NO: 51; and (c) HVR-H3 comprising the amino acid sequence of SEQ ID NO: 52. In some embodiments, the antibody comprises HVR-H3 comprising the amino acid sequence of SEQ ID NO: 52. In some embodiments, the antibody comprises HVR-H3 comprising the amino acid sequence of SEQ ID NO: 52 and HVR-L3 comprising the amino acid sequence of SEQ ID NO: 55. In some embodiments, the antibody comprises HVR-H3 comprising the amino acid sequence of SEQ ID NO: 52, HVR-L3 comprising the amino acid sequence of SEQ ID NO: 55, and HVR-H2 comprising the amino acid sequence of SEQ ID NO: 51. In some embodiments, the antibody comprises (a) HVR-H1 comprising the amino acid sequence of SEQ ID NO: 50; (b) HVR-H2 comprising the amino acid sequence of SEQ ID NO: 51; and (c) HVR-H3 comprising the amino acid sequence of SEQ ID NO: 52.

In some embodiments, an antibody is provided that comprises at least one, at least two, or all three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55. In some embodiments, the antibody comprises (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In some embodiments, an antibody comprises (a) a VH domain comprising at least one, at least two, or all three VH HVR sequences selected from (i) HVR-H1 comprising the amino acid sequence of SEQ ID NO: 50, (ii) HVR-H2 comprising the amino acid sequence of SEQ ID NO: 51, and (iii) HVR-H3 comprising an amino acid sequence selected from SEQ ID NO: 52; and (b) a VL domain comprising at least one, at least two, or all three VL HVR sequences selected from (i) HVR-L1 comprising the amino acid sequence of SEQ ID NO: 53, (ii) HVR-L2 comprising the amino acid sequence of SEQ ID NO: 54, and (c) HVR-L3 comprising the amino acid sequence of SEQ ID NO: 55.

In some embodiments, an antibody is provided that comprises (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; (d) HVR-L1 comprising the amino acid sequence of SEQ ID NO: 53; (e) HVR-L2 comprising the amino acid sequence of SEQ ID NO: 54; and (f) HVR-L3 comprising an amino acid sequence selected from SEQ ID NO: 55.

In any of the above embodiments, an anti-IL-13 antibody is humanized. In some embodiments, an anti-IL-13 antibody comprises HVRs as in any of the above embodiments, and further comprises an acceptor human framework, e.g. a human immunoglobulin framework or a human consensus framework. In some embodiments, an anti-IL-13antibody comprises HVRs as in any of the above embodiments, and further comprises a VH comprising FR1, FR2, FR3, and/or FR4 sequences of SEQ ID NO: 49. In some embodiments, an anti-IL-13antibody comprises HVRs as in any of the above embodiments, and further comprises a VL comprising FR1, FR2, FR3, and/or FR4 sequences of SEQ ID NO: 48.

In some embodiments, an anti-IL-13 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 49. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 49. In certain embodiments, substitutions, insertions, or deletions occur in regions outside the HVRs (i.e., in the FRs). Optionally, the anti-IL-13 antibody comprises the VH sequence in SEQ ID NO: 49, including post-translational modifications of that sequence. In a particular embodiment, the VH comprises one, two or three HVRs selected from: (a) HVR-H1 comprising the amino acid sequence of SEQ ID NO: 50, (b) HVR-H2 comprising the amino acid sequence of SEQ ID NO: 51, and (c) HVR-H3 comprising the amino acid sequence of SEQ ID NO: 52.

In some embodiments, an anti-IL-13 antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 48. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-IL-13 antibody comprising that sequence retains the ability to bind to IL-13. In certain embodiments, a total of 1 to 10 amino acids have been substituted, inserted and/or deleted in SEQ ID NO: 48. In certain embodiments, the substitutions, insertions, or deletions occur in regions outside the HVRs (i.e., in the FRs). Optionally, the anti-IL-13 antibody comprises the VL sequence in SEQ ID NO: 48, including post-translational modifications of that sequence. In a particular embodiment, the VL comprises one, two or three HVRs selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In some embodiments, an anti-IL-13 antibody is provided, wherein the antibody comprises a VH as in any of the embodiments provided above, and a VL as in any of the embodiments provided above. In some embodiments, the antibody comprises the VH and VL sequences in SEQ ID NO: 49 and SEQ ID NO: 48, respectively, including post-translational modifications of those sequences.

In some embodiments, an antibody is provided that competes for binding to IL-13 with an anti-IL-13 antibody comprising a VH sequence of SEQ ID NO: 49 and a VL sequence of SEQ ID NO: 48. In some embodiments, an antibody is provided that binds to the same epitope as an anti-IL-13 antibody provided herein. See, e.g., Ultsch, M. et al., Structural Basis of Signaling Blockade by Anti-IL-13 Antibody Lebrikizumab, J. Mol. Biol. (2013), dx.doi.org/10.1016/j.jmb.2013.01.053. In some embodiments, an antibody is provided that binds to the same epitope as an anti-IL-13 antibody provided herein. For example, in certain embodiments, an antibody is provided that binds to the same epitope as an anti-IL-13 antibody comprising a VH sequence of SEQ ID NO: 49 and a VL sequence of SEQ ID NO: 48.

In some embodiments, an anti-IL-13 antibody according to any of the above embodiments is a monoclonal antibody, including a chimeric, humanized or human antibody. In some embodiments, an anti-IL-13 antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-13 antibody according to any of the above embodiments may incorporate any of the features, singly or in combination, as described in Sections 1-7 below.

### Exemplary Anti-IL-4/IL-13 Bispecific Antibodies

In some embodiments, a multispecific antibody (such as a bispecific antibody) comprising an antigen-binding domain that specifically binds to IL-4 and IL-13 is provided. In some embodiments, the antigen-binding domain does not specifically bind to other targets. The multispecific antibody that binds IL-4 and IL-13 may comprise a first set of variable regions (VH and VL; also referred to as a VH/VL unit) according to any of the embodiments described herein for anti-IL-4 antibodies, and a second set of variable regions (VH and VL; also referred to as a VH/VL unit) according to any of the embodiments described herein for anti-IL-13 antibodies.

In some embodiments, the multispecific antibody comprises an antigen-binding domain that specifically binds to IL-4 and IL-13 where the antibody comprises a first VH/VL unit comprising a VH (heavy chain variable domain) comprising the amino acid sequence of SEQ ID NO: 9. In some embodiments, the multispecific antibody comprises an antigen-binding domain that specifically binds to IL-4 and IL-13 where the antibody comprises a first VH/VL unit comprising a VL (light chain variable domain) comprising the amino acid sequence of SEQ ID NO: 10. In some embodiments, the multispecific antibody comprises an antigen-binding domain that specifically binds to IL-4 and IL-13 where the antibody comprises a first VH/VL unit comprising a VH comprising the amino acid sequence of SEQ ID NO: 9 and a VL comprising the amino acid sequence of SEQ ID NO: 10. In some embodiments, the multispecific antibody comprises an antigen-binding domain that specifically binds to IL-4 and IL-13 where the antibody comprises a first VH/VL unit that competes for binding to IL-4 with an antibody comprising a VH comprising the amino acid sequence of SEQ ID NO: 9 and a VL comprising the amino acid sequence of SEQ ID NO: 10.

In some embodiments, the multispecific antibody comprises an antigen-binding domain that specifically binds to IL-4 and IL-13 where the antibody comprises a second VH/VL unit comprising a VH (heavy chain variable domain) comprising the amino acid sequence of SEQ ID NO: 19 or SEQ ID NO: 56. In some embodiments, the multispecific antibody comprises an antigen-binding domain that specifically binds to IL-4 and IL-13 where the antibody comprises a second VH/VL unit comprising a VL (light chain variable domain) comprising the amino acid sequence of SEQ ID NO: 20 or SEQ ID NO: 57. In some embodiments, the multispecific antibody comprises an antigen-binding domain that specifically binds to IL-4 and IL-13 where the antibody comprises a second VH/VL unit comprising a VH comprising the amino acid sequence of SEQ ID NO: 19 or SEQ ID NO: 56 and a VL comprising the amino acid sequence of SEQ ID NO: 20 or SEQ ID NO: 57. In some embodiments, the multispecific antibody comprises an antigen-binding domain that specifically binds to IL-4 and IL-13 where the antibody comprises a second VH/VL unit that competes for binding to IL-13 with an antibody comprising a VH comprising the amino acid sequence of SEQ ID NO: 19 and a VL comprising the amino acid sequence of SEQ ID NO: 20. In some embodiments, the multispecific antibody comprises an antigen-binding domain that specifically binds to IL-4 and IL-13 where the antibody comprises a second VH/VL unit that binds an epitope of IL-13 consisting of amino acids 82 to 89 of SEQ ID NO: 29. In some embodiments, the multispecific antibody comprises an antigen-binding domain that specifically binds to IL-4 and IL-13 where the antibody comprises a second VH/VL unit that binds an epitope of IL-13 consisting of amino acids 77 to 89 of SEQ ID NO: 29.

In some embodiments, the multispecific antibody comprises an antigen-binding domain that specifically binds to IL-4 and IL-13 where the antibody comprises a second VH/VL unit comprising a VH (heavy chain variable domain) comprising the amino acid sequence of SEQ ID NO: 49. In some embodiments, the multispecific antibody comprises an antigen-binding domain that specifically binds to IL-4 and IL-13 where the antibody comprises a second VH/VL unit comprising a VL (light chain variable domain) comprising the amino acid sequence of SEQ ID NO: 48. In some embodiments, the multispecific antibody comprises an antigen-binding domain that specifically binds to IL-4 and IL-13 where the antibody comprises a second VH/VL unit comprising a VH comprising the amino acid sequence of SEQ ID NO: 49 and a VL comprising the amino acid sequence of SEQ ID NO: 48. In some embodiments, the multispecific antibody comprises an antigen-binding domain that specifically binds to IL-4 and IL-13 where the antibody comprises a second VH/VL unit that competes for binding to IL-13 with an antibody comprising a VH comprising the amino acid sequence of SEQ ID NO: 49 and a VL comprising the amino acid sequence of SEQ ID NO: 48.

In some embodiments, the multispecific antibody comprises an antigen-binding domain that specifically binds to IL-4 and IL-13 where the antibody comprises a first VH/VL unit comprising a first VH comprising the amino acid sequence of SEQ ID NO: 9 and a first VL comprising the amino acid sequence of SEQ ID NO: 10; and comprises a second VH/VL unit comprising a second VH comprising the amino acid sequence of SEQ ID NO: 19 or SEQ ID NO: 56 and a second VL comprising the amino acid sequence of SEQ ID NO: 20 or SEQ ID NO: 57.

In some embodiments, the multispecific antibody comprises an antigen-binding domain that specifically binds to IL-4 and IL-13 where the antibody comprises a first VH/VL unit comprising a first VH comprising the amino acid sequence of SEQ ID NO: 9 and a first VL comprising the amino acid sequence of SEQ ID NO: 10; and comprises a second VH/VL unit comprising a second VH comprising the amino acid sequence of SEQ ID NO: 49 and a second VL comprising the amino acid sequence of SEQ ID NO: 48.

In some embodiments, the multispecific antibody comprises an antigen-binding domain that specifically binds to IL-4 and IL-13 wherein the antibody comprises a first VH/VL unit comprising a VH having at least 90%, 91%, 92%, 93%, 94%, 95%, 96%, 97%, 98%, 99%, or 100% sequence identity to the amino acid sequence of SEQ ID NO: 9 and a VL having at least 90%, 91%, 92%, 93%, 94%, 95%, 96%, 97%, 98%, 99%, or 100% sequence identity to the amino acid sequence of SEQ ID NO: 10. In some embodiments, the multispecific antibody comprises an antigen-binding domain that specifically binds to IL-4 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 19 and a VL having at least 90%, 91%, 92%, 93%, 94%, 95%, 96%, 97%, 98%, 99%, or 100% sequence identity to the amino acid sequence of SEQ ID NO: 20. In some embodiments, the multispecific antibody comprises an antigen-binding domain that specifically binds to IL-4 and IL-13 where the antibody comprises a second VH/VL unit comprising a VH having at least 90%, 91%, 92%, 93%, 94%, 95%, 96%, 97%, 98%, 99%, or 100% sequence identity to the amino acid sequence of SEQ ID NO: 49 and a VL having at least 90%, 91%, 92%, 93%, 94%, 95%, 96%, 97%, 98%, 99%, or 100% sequence identity to the amino acid sequence of SEQ ID NO: 48. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-4 and IL-13 wherein the antibody comprises a first VH/VL unit comprising a first VH having at least 90%, 91%, 92%, 93%, 94%, 95%, 96%, 97%, 98%, 99%, or 100% sequence identity to the amino acid sequence of SEQ ID NO: 9 and a first VL having at least 90%, 91%, 92%, 93%, 94%, 95%, 96%, 97%, 98%, 99%, or 100% sequence identity to the amino acid sequence of SEQ ID NO: 10; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 19 and a second VL having at least 90%, 91%, 92%, 93%, 94%, 95%, 96%, 97%, 98%, 99%, or 100% sequence identity to the amino acid sequence of SEQ ID NO: 20. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, the multispecific antibody comprises an antigen-binding domain that specifically binds to IL-4 and IL-13 wherein the antibody comprises a first VH/VL unit comprising a first VH having at least 90%, 91%, 92%, 93%, 94%, 95%, 96%, 97%, 98%, 99%, or 100% sequence identity to the amino acid sequence of SEQ ID NO: 9 and a first VL having at least 90%, 91%, 92%, 93%, 94%, 95%, 96%, 97%, 98%, 99%, or 100% sequence identity to the amino acid sequence of SEQ ID NO: 10; and a second VH/VL unit comprising a second VH having at least 90%, 91%, 92%, 93%, 94%, 95%, 96%, 97%, 98%, 99%, or 100% sequence identity to the amino acid sequence of SEQ ID NO: 49 and a second VL having at least 90%, 91%, 92%, 93%, 94%, 95%, 96%, 97%, 98%, 99%, or 100% sequence identity to the amino acid sequence of SEQ ID NO: 48. In certain embodiments, a total of 1 to 10 amino acids have been substituted, inserted and/or deleted in the sequences above. In certain embodiments, substitutions, insertions, or deletions occur in regions outside the HVRs (i.e., in the FRs).

In some embodiments, a multispecific antibody comprises an antigen-binding domain that specifically binds to IL-4 and IL-13 where the antibody comprises a first VH/VL unit comprising at least one, two, three, four, five, or six HVRs selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising the amino acid sequence of SEQ ID NO: 17. In some embodiments, a multispecific antibody comprises an antigen-binding domain that specifically binds to IL-4 and IL-13 where the antibody comprises a second VH/VL unit comprising at least one, two, three, four, five, or six HVRs selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26. In some embodiments, a multispecific antibody comprises an antigen-binding domain that specifically binds to IL-4 and IL-13 where the antibody comprises a second VH/VL unit comprising at least one, two, three, four, five, or six HVRs selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; (d) HVR-L1 comprising the amino acid sequence of SEQ ID NO: 53; (e) HVR-L2 comprising the amino acid sequence of SEQ ID NO: 54; and (f) HVR-L3 comprising the amino acid sequence of SEQ ID NO: 55.

In some embodiments, a multispecific antibody comprises an antigen-binding domain that specifically binds to IL-4 and IL-13 where the antibody comprises a first VH/VL unit comprising at least one, two, three, four, five, or six HVRs selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising the amino acid sequence of SEQ ID NO: 17; and a second VH/VL unit comprising at least one, two, three, four, five, or six HVRs selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; (d) HVR-L1 comprising the amino acid sequence of SEQ ID NO: 24; (e) HVR-L2 comprising the amino acid sequence of SEQ ID NO: 25; and (f) HVR-L3 comprising the amino acid sequence of SEQ ID NO: 26.

In some embodiments, a multispecific antibody comprises an antigen-binding domain that specifically binds to IL-4 and IL-13 where the antibody comprises a first VH/VL unit comprising at least one, two, three, four, five, or six HVRs selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; (d) HVR-L1 comprising the amino acid sequence of SEQ ID NO: 15; (e) HVR-L2 comprising the amino acid sequence of SEQ ID NO: 16; and (f) HVR-L3 comprising the amino acid sequence of SEQ ID NO: 17; and a second VH/VL unit comprising at least one, two, three, four, five, or six HVRs selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; (d) HVR-L1 comprising the amino acid sequence of SEQ ID NO: 53; (e) HVR-L2 comprising the amino acid sequence of SEQ ID NO: 54; and (f) HVR-L3 comprising the amino acid sequence of SEQ ID NO: 55.

In some embodiments, a multispecific antibody comprises an antigen-binding domain that specifically binds to IL-4 and IL-13 where the antibody comprises a first VH/VL unit comprising at least one, at least two, or all three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14. In some embodiments, a multispecific antibody comprises an antigen-binding domain that specifically binds to IL-4 and IL-13 where the antibody comprises a second VH/VL unit comprising at least one, at least two, or all three VH HVR sequences selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23. In some embodiments, a multispecific antibody comprises an antigen-binding domain that specifically binds to IL-4 and IL-13 where the antibody comprises a second VH/VL unit comprising at least one, at least two, or all three VH HVR sequences selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52.

In some embodiments, a multispecific antibody comprises an antigen-binding domain that specifically binds to IL-4 and IL-13 where the antibody comprises a first VH/VL unit comprising at least one, at least two, or all three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; and a second VH/VL unit comprising at least one, at least two, or all three VH HVR sequences selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23. In some embodiments, a multispecific antibody comprises an antigen-binding domain that specifically binds to IL-4 and IL-13 where the antibody comprises a first VH/VL unit comprising at least one, at least two, or all three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; and a second VH/VL unit comprising at least one, at least two, or all three VH HVR sequences selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52.

In some embodiments, a multispecific antibody comprises an antigen-binding domain that specifically binds to IL-4 and IL-13 where the antibody comprises a first VH/VL unit comprising at least one, at least two, or all three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17. In some embodiments, a multispecific antibody comprises an antigen-binding domain that specifically binds to IL-4 and IL-13 where the antibody comprises a second VH/VL unit comprising at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, a multispecific antibody comprises an antigen-binding domain that specifically binds to IL-4 and IL-13 where the antibody comprises a second VH/VL unit comprising at least one, at least two, or all three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In some embodiments, a multispecific antibody comprises an antigen-binding domain that specifically binds to IL-4 and IL-13 where the antibody comprises a first VH/VL unit comprising at least one, at least two, or all three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17; and a second VH/VL unit comprising at least one, at least two, or all three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26. In some embodiments, a multispecific antibody comprises an antigen-binding domain that specifically binds to IL-4 and IL-13 where the antibody comprises a first VH/VL unit comprising at least one, at least two, or all three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17; and a second VH/VL unit comprising at least one, at least two, or all three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In some embodiments, a multispecific antibody comprises an antigen-binding domain that specifically binds to IL-4 and IL-13 where the antibody comprises a first VH/VL unit comprising three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; and three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17. In some embodiments, a multispecific antibody comprises an antigen-binding domain that specifically binds to IL-4 and IL-13 where the antibody comprises a first VH/VL unit comprising three VH HVR sequences selected from (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; and three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17.

In some embodiments, a multispecific antibody comprises an antigen-binding domain that specifically binds to IL-4 and IL-13 where the antibody comprises a second VH/VL unit comprising three VH HVR sequences selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; and three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, a multispecific antibody comprises an antigen-binding domain that specifically binds to IL-4 and IL-13 where the antibody comprises a second VH/VL unit comprising three VH HVR sequences selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; and three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In some embodiments, a multispecific antibody comprises an antigen-binding domain that specifically binds to IL-4 and IL-13 where the antibody comprises a first VH/VL unit comprising three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; and three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17; and a second VH/VL unit comprising three VH HVR sequences selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; and three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, a multispecific antibody comprises an antigen-binding domain that specifically binds to IL-4 and IL-13 where the antibody comprises a first VH/VL unit comprising three VH HVR sequences selected from (a) HVR-H1 comprising the amino acid sequence of SEQ ID NO: 12; (b) HVR-H2 comprising the amino acid sequence of SEQ ID NO: 13 or SEQ ID NO: 18; (c) HVR-H3 comprising the amino acid sequence of SEQ ID NO: 14; and three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17; and a second VH/VL unit comprising three VH HVR sequences selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; and three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In some embodiments, a multispecific antibody comprises an antigen-binding domain that specifically binds to IL-4 and IL-13 where the antibody comprises a first VH/VL unit comprising three VH HVR sequences selected from (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; and three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17; and a second VH/VL unit comprising three VH HVR sequences selected from (a) HVR-H1 comprising the amino acid sequence of SEQ ID NO: 21 or SEQ ID NO: 60; (b) HVR-H2 comprising the amino acid sequence of SEQ ID NO: 22; (c) HVR-H3 comprising the amino acid sequence of SEQ ID NO: 23; and three VL HVR sequences selected from (a) HVR-L1 comprising the amino acid sequence of SEQ ID NO: 24; (b) HVR-L2 comprising the amino acid sequence of SEQ ID NO: 25; and (c) HVR-L3 comprising the amino acid sequence of SEQ ID NO: 26.

In some embodiments, a multispecific antibody comprises an antigen-binding domain that specifically binds to IL-4 and IL-13 where the antibody comprises a first VH/VL unit comprising three VH HVR sequences selected from (a) HVR-H1 comprising the amino acid sequence of SEQ ID NO: 12; (b) HVR-H2 comprising the amino acid sequence of SEQ ID NO: 13; (c) HVR-H3 comprising the amino acid sequence of SEQ ID NO: 14; and three VL HVR sequences selected from (a) HVR-L1 comprising the amino acid sequence of SEQ ID NO: 15; (b) HVR-L2 comprising the amino acid sequence of SEQ ID NO: 16; and (c) HVR-L3 comprising the amino acid sequence of SEQ ID NO: 17; and a second VH/VL unit comprising three VH HVR sequences selected from (a) HVR-H1 comprising the amino acid sequence of SEQ ID NO: 50; (b) HVR-H2 comprising the amino acid sequence of SEQ ID NO: 51; (c) HVR-H3 comprising the amino acid sequence of SEQ ID NO: 52; and three VL HVR sequences selected from (a) HVR-L1 comprising the amino acid sequence of SEQ ID NO: 53; (b) HVR-L2 comprising the amino acid sequence of SEQ ID NO: 54; and (c) HVR-L3 comprising the amino acid sequence of SEQ ID NO: 55.

In various embodiments, a multispecific antibody comprises a first hemimer comprising a first VH/VL unit that binds IL-4, wherein the first hemimer comprises a knob mutation in the heavy chain constant region, and a second hemimer comprising a second VH/VL unit that binds IL-13, wherein the second hemimer comprises a hole mutation in the heavy chain constant region. In various embodiments, a multispecific antibody comprises a first hemimer comprising a first VH/VL unit that binds IL-4, wherein the first hemimer comprises a hole mutation in the heavy chain constant region, and a second hemimer comprising a second VH/VL unit that binds IL-13, wherein the second hemimer comprises a knob mutation in the heavy chain constant region. In some embodiments, a heavy chain constant region comprising a hole mutation has the sequence shown in SEQ ID NO: 35 (IgG1) or SEQ ID NO: 37 (IgG4). In some embodiments, a heavy chain constant region comprising a knob mutation has the sequence shown in SEQ ID NO: 34 (IgG1) or SEQ ID NO: 36 (IgG4). In some embodiments, a multispecific antibody comprises a first hemimer comprising a first heavy chain having the sequence of SEQ ID NO: 38 and a first light chain having the sequence of SEQ ID NO: 39, and a second hemimer comprising a second heavy chain having the sequence of SEQ ID NO: 40 or 58 and a second light chain having the sequence of SEQ ID NO: 41 or 59. In some embodiments, a multispecific antibody comprises a first hemimer comprising a first heay chain having the sequence of SEQ ID NO: 38 and a first light chain having the sequence of SEQ ID NO: 39, and a second hemimer comprising a second heavy chain having the sequence of SEQ ID NO: 40 and a second light chain having the sequence of SEQ ID NO: 41.

In some embodiments, an anti-IL-4/IL-13 multispecific antibody according to any of the above embodiments is a monoclonal antibody, including a chimeric, humanized or human antibody. In some embodiments, an anti-IL-4/IL-13 multispecific antibody is an antibody fragment, e.g., a Fv, Fab, Fab′, scFv, diabody, or F(ab′)₂ fragment. In some embodiments, the antibody is a full length antibody, e.g., an intact IgG1 or IgG4 antibody or other antibody class or isotype as defined herein.

In some embodiments, an anti-IL-4/IL-13 multispecific antibody according to any of the above embodiments may incorporate any of the features, singly or in combination, as described in Sections 1-7 below.

### 1. Antibody Affinity

In certain embodiments, an antibody provided herein has a dissociation constant (Kd) for an antigen of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹ M to 10⁻¹³ M).

In some embodiments, Kd is measured by a radiolabeled antigen binding assay (RIA). In some embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to some embodiments, Kd is measured using a BIACORE® surface plasmon resonance assay. For example, an assay using a BIACORE®-2000 or a BIACORE® 3000 (BIAcore, Inc., Piscataway, N.J.) is performed at 25° C. with immobilized antigen CM5 chips at ˜10 response units (RU). In some embodiments, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensograms. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE′ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies described herein may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N. J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N. J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284 (1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for IL-4 and the other is for any other antigen. In certain embodiments, one of the binding specificities is for IL-4 and the other is IL-13. In certain embodiments, bispecific antibodies may bind to two different epitopes of IL-4. Bispecific antibodies may also be used to localize cytotoxic agents to cells. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; U.S. Publication No. 2011/0287009). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using a furin cleavable tether between a C_{L} domain and a V_{H} domain in a single VH/VL unit (see, e.g., International Patent App. No. PCT/US2012/059810); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to IL-4 as well as another, different antigen, such as IL-13 (see, US 2008/0069820, for example).

Knobs into Holes

The use of knobs into holes as a method of producing multispecific antibodies is described, e.g., in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9. A brief nonlimiting discussion is provided below.

A “protuberance” refers to at least one amino acid side chain which projects from the interface of a first polypeptide and is therefore positionable in a compensatory cavity in the adjacent interface (i.e. the interface of a second polypeptide) so as to stabilize the heteromultimer, and thereby favor heteromultimer formation over homomultimer formation, for example. The protuberance may exist in the original interface or may be introduced synthetically (e.g. by altering nucleic acid encoding the interface). In some embodiments, nucleic acid encoding the interface of the first polypeptide is altered to encode the protuberance. To achieve this, the nucleic acid encoding at least one “original” amino acid residue in the interface of the first polypeptide is replaced with nucleic acid encoding at least one “import” amino acid residue which has a larger side chain volume than the original amino acid residue. It will be appreciated that there can be more than one original and corresponding import residue. The side chain volumes of the various amino residues are shown, for example, in Table 1 of US2011/0287009.

In some embodiments, import residues for the formation of a protuberance are naturally occurring amino acid residues selected from arginine (R), phenylalanine (F), tyrosine (Y) and tryptophan (W). In some embodiments, an import residue is tryptophan or tyrosine. In some embodiment, the original residue for the formation of the protuberance has a small side chain volume, such as alanine, asparagine, aspartic acid, glycine, serine, threonine or valine.

A “cavity” refers to at least one amino acid side chain which is recessed from the interface of a second polypeptide and therefore accommodates a corresponding protuberance on the adjacent interface of a first polypeptide. The cavity may exist in the original interface or may be introduced synthetically (e.g. by altering nucleic acid encoding the interface). In some embodiments, nucleic acid encoding the interface of the second polypeptide is altered to encode the cavity. To achieve this, the nucleic acid encoding at least one “original” amino acid residue in the interface of the second polypeptide is replaced with DNA encoding at least one “import” amino acid residue which has a smaller side chain volume than the original amino acid residue. It will be appreciated that there can be more than one original and corresponding import residue. In some embodiments, import residues for the formation of a cavity are naturally occurring amino acid residues selected from alanine (A), serine (S), threonine (T) and valine (V). In some embodiments, an import residue is serine, alanine or threonine. In some embodiments, the original residue for the formation of the cavity has a large side chain volume, such as tyrosine, arginine, phenylalanine or tryptophan.

The protuberance is “positionable” in the cavity which means that the spatial location of the protuberance and cavity on the interface of a first polypeptide and second polypeptide respectively and the sizes of the protuberance and cavity are such that the protuberance can be located in the cavity without significantly perturbing the normal association of the first and second polypeptides at the interface. Since protuberances such as Tyr, Phe and Trp do not typically extend perpendicularly from the axis of the interface and have preferred conformations, the alignment of a protuberance with a corresponding cavity may, in some instances, rely on modeling the protuberance/cavity pair based upon a three-dimensional structure such as that obtained by X-ray crystallography or nuclear magnetic resonance (NMR). This can be achieved using widely accepted techniques in the art.

In some embodiments, a knob mutation in an IgG1 constant region is T366W. In some embodiments, a hole mutation in an IgG1 constant region comprises one or more mutations selected from T366S, L368A and Y407V. In some embodiments, a hole mutation in an IgG1 constant region comprises T366S, L368A and Y407V. SEQ ID NO: 34 shows an exemplary IgG1 constant region with a knob mutation and SEQ ID NO: 35 shows an exemplary IgG1 constant region with a hole mutation.

In some embodiments, a knob mutation in an IgG4 constant region is T366W. In some embodiments, a hole mutation in an IgG4 constant region comprises one or more mutations selected from T366S, L368A, and Y407V. In some embodiments, a hole mutation in an IgG4 constant region comprises T366S, L368A, and Y407V. SEQ ID NO: 36 shows an exemplary IgG4 constant region with a knob mutation and SEQ ID NO: 37 shows an exemplary IgG4 constant region with a hole mutation.

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Conservative |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody provided herein may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, and antibody constant region, such as a heavy chain constant region, comprises a knob mutation and/or a hole mutation to facilitate formation of a multispecific antibody. Nonlimiting exemplary knob mutations and hole mutations, and knob-into-hole technology generally, are described, for example, in U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9. Certain nonlimiting exemplary knob mutations and hole mutations are discussed herein.

In certain embodiments, an antibody variant that possesses some but not all effector functions is provided, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, e.g., Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Intl. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) Clq binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

In some embodiments, an antibody constant region comprises more than one of the mutations discussed herein (for example, a knob and/or hole mutation and/or a mutation that increases stability and/or a mutation that decreases ADCC, etc.).

### Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some embodiments, conjugates of an antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In some embodiments, isolated nucleic acid encoding an anti-IL-4 antibody described herein is provided. In some embodiments, isolated nucleic acid encoding an anti-IL-13 antibody described herein is provided. In some embodiments, isolated nucleic acid encoding an anti-IL-4/IL-13 bispecific/antibody described herein is provided. Such nucleic acids may encode an amino acid sequence comprising the VL and/or an amino acid sequence comprising the VH of the antibody (e.g., the light and/or heavy chains of the antibody). In some embodiments, one or more vectors (e.g., expression vectors) comprising such nucleic acid are provided. In some embodiments, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

In some embodiments, the host cell is eukaryotic, e.g. a Chinese Hamster Ovary (CHO) cell or lymphoid cell (e.g., Y0, NS0, Sp20 cell). In some embodiments, a method of making an antibody is provided, wherein the method comprises culturing a host cell comprising nucleic acid encoding the antibody, as provided above, under conditions suitable for expression of the antibody, and optionally recovering the antibody from the host cell (or host cell culture medium).

In some embodiments, a method of making a multispecific antibody is provided, wherein the method comprises culturing in a host cell comprising nucleic acid encoding the multispecific antibody under conditions suitable for expression of the antibody, and optionally recovering the multispecific antibody from the host cell (or host cell culture medium). In some embodiments, a method of making a multispecific antibody is provided, wherein the method comprises culturing a first host cell comprising nucleic acid encoding a first VH/VL unit of the multispecific antibody (including constant region, if any, sometimes referred to as a “hemimer” or “half-antibody”) under conditions suitable for expression of the first VH/VL unit, and optionally recovering the first VH/VL unit from the host cell (or host cell culture medium), and culturing a second host cell comprising nucleic acid encoding a second VH/VL unit of the multispecific antibody (including constant region, if any) under conditions suitable for expression of the second VH/VL unit, and optionally recovering the second VH/VL unit from the host cell (or host cell culture medium). In some embodiments, the method further comprises assembling the multispecific antibody from an isolated first VH/VL unit and an isolated second VH/VL unit. Such assembly may comprise, in some embodiments, a redox step to form intramolecular disulfides between the two VH/VL units (or hemimers). Nonlimiting exemplary methods of producing multispecific antibodies are described, e.g., in US 2011/0287009, US 2007/0196363, US2007/0178552, U.S. Pat. No. 5,731,168, WO 96/027011, WO 98/050431, and Zhu et al., 1997, Protein Science 6:781-788. A nonlimiting exemplary method is also described in the examples below.

For recombinant production of an anti-IL-4 antibody or anti-IL-4/IL-13 bispecific antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N. J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498; 6,420,548; 7,125,978; and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Exemplary Assays

### Binding Assays and Other Assays

In some embodiments, an antibody provided herein is tested for its antigen binding activity, e.g., by known methods such as ELISA, Western blot, etc.

In some embodiments, competition assays may be used to identify an antibody that competes with an IL-4 antibody described herein for binding to IL-4. In some embodiments, competition assays may be used to identify an antibody that competes with an IL-4/IL-13 bispecific antibody described herein for binding to IL-4 and/or IL-13. In certain embodiments, such a competing antibody binds to the same epitope (e.g., a linear or a conformational epitope) that is bound by an antibody that comprises a VH amino acid sequence comprising SEQ ID NO: 9 and a VL amino acid sequence comprising SEQ ID NO: 10 for binding IL-4. In certain embodiments, such a competing antibody binds to the same epitope (e.g., a linear or a conformational epitope) that is bound by an antibody that comprises a VH amino acid sequence comprising SEQ ID NO: 19 and a VL amino acid sequence comprising SEQ ID NO: 20 for binding IL-13. In certain embodiments, such a competing antibody binds to the same epitope (e.g., a linear or a conformational epitope) that is bound by an antibody that comprises a VH amino acid sequence comprising SEQ ID NO: 49 and a VL amino acid sequence comprising SEQ ID NO: 48 for binding IL-13. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized IL-4 is incubated in a solution comprising a first labeled antibody that binds to IL-4 (e.g., an antibody that comprises a VH amino acid sequence comprising SEQ ID NO: 9 and a VL amino acid sequence comprising SEQ ID NO: 10) and a second unlabeled antibody that is being tested for its ability to compete with the first antibody for binding to IL-4. The second antibody may be present in a hybridoma supernatant. As a control, immobilized IL-4 is incubated in a solution comprising the first labeled antibody but not the second unlabeled antibody. After incubation under conditions permissive for binding of the first antibody to IL-4, excess unbound antibody is removed, and the amount of label associated with immobilized IL-4 is measured. If the amount of label associated with immobilized IL-4 is substantially reduced in the test sample relative to the control sample, then that indicates that the second antibody is competing with the first antibody for binding to IL-4. See Harlow and Lane (1988) Antibodies: A Laboratory Manual ch. 14 (Cold Spring Harbor Laboratory, Cold Spring Harbor, N.Y.).

In a further exemplary competition assay, immobilized IL-13 is incubated in a solution comprising a first labeled antibody that binds to IL-13 (e.g., an antibody that comprises a VH amino acid sequence comprising SEQ ID NO: 19 and a VL amino acid sequence comprising SEQ ID NO: 20, or an antibody that comprises a VH amino acid sequence comprising SEQ ID NO: 49 and a VL amino acid sequence comprising SEQ ID NO: 48) and a second unlabeled antibody that is being tested for its ability to compete with the first antibody for binding to IL-13. The second antibody may be present in a hybridoma supernatant. As a control, immobilized IL-13 is incubated in a solution comprising the first labeled antibody but not the second unlabeled antibody. After incubation under conditions permissive for binding of the first antibody to IL-13, excess unbound antibody is removed, and the amount of label associated with immobilized IL-13 is measured. If the amount of label associated with immobilized IL-13 is substantially reduced in the test sample relative to the control sample, then that indicates that the second antibody is competing with the first antibody for binding to IL-13.

### Activity Assays

In some embodiments, assays are provided for identifying anti-IL-4 antibodies and anti-IL-4/IL-13 bispecific antibodies having biological activity. Biological activity may include, e.g., inhibition of IL-4 binding to an IL-4 receptor, inhibition of IL-4-induced STAT6 phosphorylation, inhibition of IL-4 induced cell proliferation, inhibition of IL-4-induced class switching of B cells to IgE, activity in asthma, and activity in IPF. In some embodiments, biological activities include, e.g., inhibition of IL-13 binding to an IL-13 receptor (for example, a heterodimeric receptor comprising IL-4Rα and IL-13Rα1), inhibition of IL-13-induced STAT6 phosphorylation, inhibition of IL-13-induced cell proliferation, inhibition of IL-13-induced class switching of B cells to IgE, inhibition of IL-13-induced mucus production, activity in asthma, and activity in IPF. Antibodies having such biological activity in vivo and/or in vitro are also provided. Nonlimiting exemplary assays for testing for such biological activities are described herein and/or are known in the art.

### Immunoconjugates

In some embodiments, immunoconjugates comprising an anti-IL-4 antibody or an anti-IL-4/IL-13 bispecific antibody conjugated to one or more cytotoxic agents is provided. Nonlimiting exemplary such cytotoxic agents include chemotherapeutic agents or drugs, growth inhibitory agents, toxins (e.g., protein toxins, enzymatically active toxins of bacterial, fungal, plant, or animal origin, or fragments thereof), and radioactive isotopes.

In some embodiments, an immunoconjugate is an antibody-drug conjugate (ADC) in which an antibody is conjugated to one or more drugs, including but not limited to a maytansinoid (see, e.g., U.S. Pat. Nos. 5,208,020, 5,416,064 and European Patent EP 0 425 235 B1); an auristatin such as monomethylauristatin drug moieties DE and DF (MMAE and MMAF) (see, e.g., U.S. Pat. Nos. 5,635,483 and 5,780,588, and 7,498,298); a dolastatin; a calicheamicin or derivative thereof (see, e.g., U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see, e.g., Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAM, and PAP-S), momordica charantia inhibitor, curcin, crotin, sapaonaria officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### Methods and Compositions for Diagnostics and Detection

In certain embodiments, any of the anti-IL-4 antibodies provided herein is useful for detecting the presence of IL-4 in a biological sample. In certain embodiments, any of the anti-IL-4/IL-13 bispecific antibodies provided herein is useful for detecting the presence of IL-4 and/or IL-13 in a biological sample. The term “detecting” as used herein encompasses quantitative or qualitative detection. In certain embodiments, a biological sample comprises a cell or tissue, such as serum, plasma, nasal swabs, bronchoalveolar lavage fluid, and sputum.

In some embodiments, an anti-IL-4 antibody for use in a method of diagnosis or detection is provided. In a further aspect, a method of detecting the presence of IL-4 in a biological sample is provided. In certain embodiments, the method comprises contacting the biological sample with an anti-IL-4 antibody as described herein under conditions permissive for binding of the anti-IL-4 antibody to IL-4, and detecting whether a complex is formed between the anti-IL-4 antibody and IL-4. Such method may be an in vitro or in vivo method. In some embodiments, an anti-IL-4 antibody is used to select subjects eligible for therapy with an anti-IL-4 antibody or anti-IL-4/IL-13 bispecific antibody, or any other TH2 pathway inhibitor, e.g. where IL-4 is a biomarker for selection of patients.

In some embodiments, an anti-IL-4/IL-13 bispecific antibody for use in a method of diagnosis or detection is provided. In a further aspect, a method of detecting the presence of IL-4 and/or IL-13 in a biological sample is provided. In certain embodiments, the method comprises contacting the biological sample with an anti-IL-4/IL-13 bispecific antibody as described herein under conditions permissive for binding of the anti-IL-4/IL-13 bispecific antibody to IL-4 and/or IL-13, and detecting whether a complex is formed between the anti-IL-4/IL-13 bispecific antibody and IL-4 and/or IL-13. Such method may be an in vitro or in vivo method. In some embodiments, an anti-IL-4/IL-13 bispecific antibody is used to select subjects eligible for therapy with an anti-IL-4/IL-13 bispecific antibody, or any other TH2 pathway inhibitor, e.g. where IL-4 and/or IL-13 is a biomarker for selection of patients.

Exemplary disorders that may be diagnosed using an anti-IL-4 antibody of anti-IL-4/IL-13 bispecific antibody are provided herein.

In certain embodiments, labeled anti-IL-4 antibodies are provided. In certain embodiments, labeled anti-IL-4/IL-13 bispecific antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Pharmaceutical Formulations

Pharmaceutical formulations of an anti-IL-4 antibody and/or an anti-IL-4/IL-13 bispecific antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In some embodiments, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide a controller and/or TH2 pathway inhibitor with the anti-IL-4 antibody and/or anti-IL-4/IL-13 bispecific antibody. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### Therapeutic Methods and Compositions

Any of the anti-IL-4 antibodies provided herein may be used in therapeutic methods. Any of the anti-IL-4/IL-13 bispecific antibodies provided herein may be used in therapeutic methods.

In certain embodiments, an anti-IL-4 antibody and/or anti-IL-4/IL-13 bispecific antibody for use as a medicament is provided. In certain embodiments, an anti-IL-4 antibody and/or anti-IL-4/IL-13 bispecific antibody for use in treating asthma, IPF, a respiratory disorder, an eosinophilic disorder, an IL-13 mediated disorder, or an IL-4 mediated disorder is provided. In certain embodiments, an anti-IL-4 antibody and/or anti-IL-4/IL-13 bispecific antibody for use in a method of treatment is provided. In certain embodiments, an anti-IL-4 antibody or anti-IL-4/IL-13 bispecific antibody is provided for use in a method of treating an individual having asthma, a respiratory disorder, an eosinophilic disorder, an IL-13 mediated disorder, or an IL-4 mediated disorder comprising administering to the individual an effective amount of the anti-IL-4 antibody or anti-IL-4/IL-13 bispecific antibody. In one such embodiment, the method further comprises administering to the individual an effective amount of at least one additional therapeutic agent, e.g., as described below.

An “individual” according to any of the above embodiments is preferably a human.

In some embodiments, use of an anti-IL-4 antibody and/or an anti-IL-4/IL-13 bispecific antibody in the manufacture or preparation of a medicament is provided. In one embodiment, the medicament is for treatment of asthma, a respiratory disorder, an eosinophilic disorder, an IL-13 mediated disorder, or an IL-4 mediated disorder. In a further embodiment, the medicament is for use in a method of treating asthma, IPF, a respiratory disorder, an eosinophilic disorder, an IL-13 mediated disorder, or an IL-4 mediated disorder comprising administering to an individual having asthma, a respiratory disorder, an eosinophilic disorder, an IL-13 mediated disorder, or an IL-4 mediated disorder an effective amount of the medicament. In one such embodiment, the method further comprises administering to the individual an effective amount of at least one additional therapeutic agent, e.g., as described below.

In some embodiments, pharmaceutical formulations comprising any of the anti-IL-4 antibodies and/or anti-IL-4/IL-13 bispecific antibodies described herein are provided, e.g., for use in any of the above therapeutic methods. In some embodiments, a pharmaceutical formulation comprises any of the anti-IL-4 antibodies and/or anti-IL-4/IL-13 bispecific antibodies provided herein and a pharmaceutically acceptable carrier. In some embodiments, a pharmaceutical formulation comprises any of the anti-IL-4 antibodies and/or anti-IL-4/IL-13 bispecific antibodies provided herein and at least one additional therapeutic agent, e.g., as described below.

Antibodies provided herein can be used either alone or in combination with other agents in a therapy. For instance, an antibody provided herein may be co-administered with at least one additional therapeutic agent. In certain embodiments, an additional therapeutic agent is a TH2 inhibitor. In certain embodiments, an additional therapeutic is a controller of asthma inflammation, such as a corticosteroid, leukotriene receptor antagonist, LABA, corticosteroid/LABA combination composition, theophylline, cromolyn sodium, nedocromil sodium, omalizumab, LAMA, MABA (e.g., bifunctional muscarinic antagonist-beta2 Agonist), 5-Lipoxygenase Activating Protein (FLAP) inhibitor, or enzyme PDE-4 inhibitor.

Such combination therapies noted above encompass combined administration (where two or more therapeutic agents are included in the same or separate formulations), and separate administration, in which case, administration of the anti-IL-4 antibody and/or anti-IL-4/IL-13 bispecific antibody can occur prior to, simultaneously, and/or following, administration of the additional therapeutic agent or agents. In some embodiments, administration of the anti-IL-4 antibody and/or anti-IL-4/IL-13 bispecific antibody and administration of an additional therapeutic agent occur within about one month, or within about one, two or three weeks, or within about one, two, three, four, five, or six days, of each other.

In some embodiments, an anti-IL-4 antibody and/or anti-IL-4/IL-13 bispecific antibody is used in treating cancer, such as glioblastoma or non-Hodgkin's lymphoma. In some embodiments, antibodies provided herein can also be used in combination with radiation therapy.

An anti-IL-4 antibody and/or anti-IL-4/IL-13 bispecific antibody (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

An anti-IL-4 antibody and/or anti-IL-4/IL-13 bispecific antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an anti-IL-4 antibody and/or anti-IL-4/IL-13 bispecific antibody (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. One skilled in the art can determine a suitable dose of an antibody depending on the type and severity of the disease. Nonlimiting exemplary dosing for anti-IL-13 antibodies is described, e.g., in PCT Publication No. WO 2012/083132. General guidance for dosing of antibodies can be found, for example, in Bai et al., Clinical Pharmacokinetics, 51: 119-135 (2012) and Deng et al., Expert Opin. Drug Metab. Toxicol. 8(2):141-160 (2012). The progress of the antibody therapy may be monitored by conventional techniques and assays.

It is understood that any of the above formulations or therapeutic methods may be carried out using an immunoconjugate in place of or in addition to an anti-IL 4 antibody or anti-IL-4/IL-13 bispecific antibody.

### Articles of Manufacture

In some embodiments, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an anti-IL-4 antibody and/or anti-IL-4/IL-13 bispecific antibody. The label or package insert indicates that the composition is used for treating the condition of choice. Moreover, the article of manufacture may comprise (a) a first container with a composition contained therein, wherein the composition comprises an anti-IL-4 antibody and/or anti-IL-4/IL-13 bispecific antibody; and (b) a second container with a composition contained therein, wherein the composition comprises a further cytotoxic or otherwise therapeutic agent. In some embodiments, the article of manufacture may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate in place of or in addition to an anti-IL-4 antibody or anti-IL-4/IL-13 bispecific antibody.

### EXAMPLES

The following are examples of methods and compositions of the invention. It is understood that various other embodiments may be practiced, given the general description provided above.

### Example 1

### Certain Methods and Reagents

Surface Plasmon Resonance (SPR) BIAcore affinity measurement

The binding kinetics of the anti-IL-4, anti-IL-13 and anti-IL-4/IL-13 bispecific antibodies were measured using surface plasmon resonance (SPR) on a Biacore 3000 instrument (GE Healthcare). Anti-human Fc (GE Healthcare) was immobilized on a CM5 sensor chip via amine-based coupling using manufacturer provided protocol. Antibody was captured at a level of 1200 resonance units (RU).

Bispecific binding was measured to human IL-4, cyno IL-4, human IL-13, human IL-13 R130Q (SEQ ID NO: 31), and cyno IL-13 at concentrations of 0, 3.13, 6.25, 12.50, 25.0, and 50.0 nM. Sensograms for binding of cytokine were recorded using an injection time of 2 minutes with a flow rate of 30 μl/min, at a temperature of 25° C., and with a running buffer of 10 mM HEPES, pH 7.4, 150 mM NaCl, and 0.005% Tween 20. After injection, disassociation of the cytokine from the antibody was monitored for 1000 seconds in running buffer. The surface was regenerated between binding cycles with a 60 μl injection of 3 M Magnesium Chloride. After subtraction of a blank which contained running buffer only, sensograms observed for cytokine binding to anti-IL-13/anti-IL-4 bispecific antibody were analyzed using a 1:1 Langmuir binding model with software supplied by the manufacturer to calculate the kinetics and binding constants.

### Surface Plasmon Resonance (SPR) BIAcore Binding Competition Assay

Inhibition of human IL-13Rα2 binding to human IL-13 by anti-IL-4/IL-13 bispecific antibody was tested using surface plasmon resonance (SPR) measurements on a Biacore 3000 instrument (GE Healthcare). Human IL-13 was immobilized on a CM5 sensor chip using the manufacturer's protocol for amine-based coupling. IL-13 was immobilized at a level of 985 resonance units (RU) on flow cell 4 (FC4), and unreacted sites were subsequently blocked using 1 M ethanolamine-HCl. FC3 was used as a reference cell for measurements, and it was prepared by activation followed by subsequent blocking with ethanolamine. Sensograms for binding of IL-13Rα2 (histidine-tagged recombinant human IL-13Rα2 made and purified according to standard methods in the art) were recorded using an injection time of 2 minutes with a flow rate of 30 μl/min, at a temperature of 25° C., and with a running buffer of 10 mM HEPES, pH 7.4, 150 mM NaCl, and 0.005% Tween 20. To determine the binding constant for IL-13Rα2 binding to IL-13, sensograms for a series of solutions of IL-13Rα2 varying in concentration (2-fold dilutions) from 12.5 to 200 nM were recorded. After injection, disassociation of the receptor from the cytokine was monitored for 600 seconds in running buffer. The surface was regenerated between binding cycles with a 60 μl injection of 10 mM Glycine-HCl pH 1.7.

To assess the binding of IL-13Rα2 to IL-13 in the presence of anti-IL-4/IL-13 bispecific antibody, an injection of 60 μl of 250 nM anti-IL-4/IL-13 bispecific antibody was added as an additional step to assess the binding of receptor to cytokine in the presence of competing antibody. After subtraction of a blank which contained running buffer only, sensograms observed for receptor binding to cytokine in the absence and presence of competing antibody were analyzed using a 1:1 Langmuir binding model with software supplied by the manufacturer to calculate the kinetics and binding constants.

### ELISA Binding Competition Assay

To determine whether an antibody inhibits IL-4 binding to IL-4 receptor (IL-4R), an ELISA assay was used. In a 96 well plate, a 150 μg/mL (1000 nM) solution of the antibody was serially diluted three fold in assay buffer (phosphate buffered saline [PBS], pH 7.5, containing 0.05% Tween 20 and 0.5% bovine serum albumin [BSA]) to provide a range of 0.0009, 0.003, 0.008, 0.02, 0.07, 0.21, 0.62, 1.9, 5.6, 16.7, 50.0, and 150 μg/mL (0.0056, 0.017, 0.05, 0.15, 0.46, 1.37, 4.12, 12.3, 37, 111, 333, and 1000 nM, respectively). The volume of each dilution was 35 μL. To each well, 35 μL of a 11.6 ng/mL (780 pM) solution of biotinylated IL 4 was added. The mixture was incubated for 40 minutes at room temperature. Following incubation, the contents of the well were transferred to a 96 well Nunc Maxisorp plate (Roskilde, Denmark) that was coated overnight with 50 μL of a 2.0 μg/mL solution of soluble IL-4R protein (R&D Systems, Cat. No. 230-4R/CF) in PBS and blocked with PBS containing 1% BSA. After a 40 minute incubation, the plate was washed five times in wash buffer (1×PBS containing 0.05% Tween 20). Each well then received 50 μL of a streptavidin horseradish peroxidase solution (Caltag Laboratories, Invitrogen; Carlsbad, Calif.) and was incubated for 40 minutes. Following five washes with wash buffer, 50 μL of tetramethylbenzidine (TMB) substrate (KPL; Gaithersburg, Md.) was added to each well. After several minutes, 50 μL of a 1 N solution of HCl was added to stop the reaction. The plate was read at 450 nM using a Spectra Max 340 plate reader (Molecular Devices; Sunnyvale, Calif.). For each sample, the optical density (OD) reading at 450 nM was plotted against concentration. Curves were plotted in Kaleidagraph (Synergy Software; Reading, Pa.) and fitted using a 4 parameter fit or plotted point to point.

To determine whether an antibody inhibits IL-13 binding to IL-13Rα1 receptor, an ELISA assay was carried out substantially as described above, except biotinylated IL-13 R130Q (SEQ ID NO: 31) was used in place of biotinylated IL-4, and soluble IL-13Rα1-Fc protein (R&D Systems, Cat. No. 146-IR-100) was used in place of soluble IL-4R-Fc.

To determine whether an antibody inhibits IL-13 binding to IL-13Rα2 receptor, an ELISA assay was carried out substantially as described above, except biotinylated IL-13 was used in place of biotinylated IL-4, and soluble IL-13Rα2-Fc protein (R&D Systems, Cat. No. 614-IR-100) was used in place of soluble IL-4R-Fc.

### Plasmid Construction and Expression of Antibodies

Antibodies were cloned into expression vectors described previously (Simmons et al., 2002, J Immunol Methods 263, 133-147). The STII signal sequence with a translation initiation strength of one for both the heavy chain and light chain preceded the sequence coding for the mature antibody. For protein expression an overnight culture in a suitable W3110 derivative (Reilly and Yansura, 2010, Antibody Engineering (Berlin, Heidelberg: Springer Berlin Heidelberg)) was grown at 30° C. in LB (100 μg/ml carbenicillin), diluted 1:100 into CRAP media (100 μg/ml carbenicillin) and grown for 24 hours at 30° C. For larger preparations, cultures were grown in 10 L fermenters, e.g., as previously described (Simmons et al., 2002, J Immunol Methods 263, 133-147).

For SDS-PAGE analysis under non-reducing conditions 2000 of overnight culture was harvested and resuspended in 100 μl of NR-lysis buffer (880 PopCulture Reagent (Novagen), 10 μl 100 mM iodoacetamide, 2 μl lysonase reagent (EMD Biosciences)). After incubation for 10 minutes at room temperature, samples were spun for 2′ at 9300 rcf and 500 supernatant transferred into a fresh tube and mixed with the same volume of 2×SDS sample buffer (Invitrogen). Before loading 10 μl of the sample on NuPAGE 4-12% Bis-Tris/MES gels (Invitrogen), samples were heated for 5′ at 95° C. and spun for 1′ at 16000 rcf. Gels were transferred by iBlot (Invitrogen) onto nitrocellulose membrane, immunoblotted with IRDye800CW conjugated anti-Human IgG F(c) antibody (Rockland) and imaged with a LiCOR Odyssey Imager.

For total reduced cell samples, the cell pellet was resuspended in R-lysis buffer (10 μM DTT, 88 μl PopCulture Reagent (Novagen), 2 μl lysonase) and incubated for 10 minutes at room temperature before samples were mixed with 2×SDS sample buffer. Western blots were images as described before with the exception that IRDye800CW conjugated anti-human antibody (Rockland) was used for immunodetection.

### Purification and Assembly of Bispecific Antibodies

E. coli whole cell broth was homogenized using a Niro-Soavi homogenizer from GEA (Bedford, N.H., U.S.A). The resulting homogenate was then extracted by addition of polyethyleneimine flocculent to a final concentration of 0.4%, diluted with purified water and mixed for 16 hours at room temperature. The extract was cleared by centrifugation and after filtration using a 0.2 μm sterile filter cooled to 15° C. and loaded on a pre-equilibrated (25 mM Tris, 25 mM NaCl 5 mM EDTA pH 7.1) Protein A column. The column was washed with equilibration buffer and 0.4 M potassium phosphate pH 7.0 and finally eluted with 100 mM acetic acid pH 2.9. The Protein A pools were then combined in an assembly reaction.

The separate half antibody Protein A pools were conditioned with 0.2 M arginine, pH adjusted using 1.5 M Tris base to pH 8.0, combined and L-reduced glutathione (GSH) was added in a 200× molar excess over bispecific antibody and incubated at 20° C. for 48 hours. After incubation, the assembled bispecific was purified by an anion exchange chromatography step and a cation exchange chromatography step. The cation exchange eluate was concentrated and buffer exchanged into final formulation buffer.

### Analytical Characterization of Antibodies by Intact and Reduced Mass Spectrometric Analysis

Reduced and intact masses of bispecifics were obtained by LC/MS analysis using an Agilent 6210 ESI-TOF mass spectrometer coupled with a nano-Chip-LC system. The bispecific samples, with and without prior TCEP reduction, at about 5 ng antibodies per injection, were desalted by RP-HPLC for direct online MS analysis. The resulting spectra for both reduced and non-reduced samples exhibited a distribution of multiply charged protein ions and the spectra were deconvoluted to zero charge state using the MassHunter Workstation software/Qualitative Analysis B.03.01 (Agilent Technologies Inc. 2009).

### Analytical Size-Exclusion Chromatography

Size variants were separated using a TosoHaas TSK G3000SW_{XL} column (7.8×300 mm) eluted isocratically with a mobile phase consisting of 0.2 M potassium phosphate and 0.25 M potassium chloride (pH 6.2). The separation was conducted at room temperature with a flow rate of 0.5 mL/min. The column effluent was monitored at 280 nm. Relative percentage of peak areas for high molecular weight species (HMWS), main peak, and low molecular weight species (LMWS) was performed by using the Chromeleon Software v6.80 SR11 from Dionex Corporation.

### Capillary Electrophoresis-Sodium Dodecyl Sulfate Analysis (CE-SDS)

The bispecific samples were first diluted with citrate-phosphate buffer pH 6.6 and treated with SDS and N-ethylmaleimide at 70° C. for 3 minutes. Upon cooling, samples were labeled at 50° C. for 10 minutes with 3-(2-furoyl)quinoline-2-carboxaldehyde) (FQ) in the presence of excess potassium cyanide. The labeling reaction was quenched by buffer exchange then treated with 1% SDS. Non-reduced samples were heated at 70° C. for 5 minutes. Reduced samples were treated with 50 mM Dithiothreitol (DTT) at 70° C. for 10 minutes.

Both non-reduced and reduced samples were analyzed by CE-SDS using a Beckman PA 800 CE system with a 50 μm diameter uncoated fused-silica capillary. Samples were injected electrokinetically (40 seconds at 5 kV), and separation was performed at a constant voltage of 15 kV in reversed polarity for 35 minutes. Capillary temperature was maintained at 40° C. The migration of labeled components was monitored by LIF detection; the excitation was at 488 nm, and the emission was monitored at 600 nm.

### Cell Culture (TF-1 Cells)

Human TF-1 (erythroleukemic cells, R&D Systems, Minneapolis, Minn.) were cultured in a humidified incubator at 37° C. with 5% CO2 in growth media containing RPMI 1640 (Genentech Media Preparation Facility, South San Francisco, Calif.) containing 10% heat inactivated fetal bovine serum (FBS) (Catalog No. SH30071.03, HyClone Laboratories, Inc., Logan, Utah); and 1× Penicillin:Streptomycin:Glutamine (Catalog No. 10378-016, Gibco Invitrogen Corp., Carlsbad, Calif.) and 2 ng/mL rhGM-CSF (Catalog No. 215-GM, R&D Systems, Minneapolis, Minn.). Assay media is growth media without 2 ng/mL rhGM-CSF. Cytokines were added to the assay media as specified, at the following final concentrations: 0.2 ng/ml human IL-4 (Catalog No. 204-IL, R&D Systems, Minneapolis, Minn.), 10 ng/ml human IL-13 (Genentech, So. San Francisco, Calif.), 10 ng/ml human IL-13 R130Q (Genentech, So. San Francisco, Calif.), 2 ng/ml cynomolgus monkey IL-4 (Genentech, So. San Francisco, Calif.), and 20 ng/ml cynomolgus monkey IL-13 (Genentech, So. San Francisco, Calif.).

### Example 2

### Generation of Antibodies the Bind IL-4

A panel of antibodies that selectively bind human interleukin-4 (IL-4) was generated using commercially-available human IL-4 (R&D Systems, Minneapolis, Minn.). Each hind footpad of 5 BALB/c mice was injected with 0.5 μg IL-4 resuspended in 25 μl total of monophosphoryl-lipid A and trehalose dicorynomycolate (MPL™+TDM)-based adjuvant (Corixa, Hamilton, Mont.) in phosphate-buffered saline (PBS) at 3- to 4-day intervals. Serum samples were taken after 7 boosts and titers determined by enzyme-linked immunosorbant assay (ELISA) to identify mice with a positive immune response to IL-4. Animals were boosted twice more via footpad (0.5 μg in 25 μl/footpad), intraperitoneal cavity (2 μg in 100 μl), and intravenous (1 μg in 50 μl) routes using adjuvant in PBS. Three days after the final boost, animals which showed positive serum titers by ELISA were sacrificed, and a single cell suspension of splenocytes was fused with the mouse myeloma cell line P3X63Ag.U.1 (American Type Culture Collection, Manassas, Va.) using electrofusion (Cyto Pulse Sciences, Inc., Glen Burnie, Md.). Fused hybridoma cells were selected from unfused splenic, popliteal node or myeloma cells using hypoxanthin-aminopterin-thymidine (HAT) selection in Medium D from the ClonaCell® hybridoma selection kit (StemCell Technologies, Inc., Vancouver, BC, Canada). Hybridoma cells were cultured in Medium E from the ClonaCell® hybridoma selection kit, and cell culture supernatants were used for further characterization and screening. To screen the 1921 hybridoma cell lines generated, enzyme-linked immunosorbant assay (ELISA) was performed generally as described earlier (Baker, K. N., et al., Trends Biotechnol. 20, 149-156 (2002)).

We identified clone 19C11, which bound to human IL-4 with an affinity of ≦10 pM, as determined by surface plasmon resonance (SPR) analysis. To determine whether 19C11 blocks binding of human IL-4 to IL-4Rα, biotinylated IL-4 (0.17 nM) was premixed with 50 μl of serially diluted supernatants of IgG (1000, 200, 40, 8, 1.6, and 0.32 nM, final concentration) from clone 19C11 or a control antibody. Following a 30 minute incubation at room temperature, the mixture was transferred to a Nunc Maxisorp plate containing immobilized soluble human IL-4Rα (R&D Systems, Minneapolis, Minn.). For immobilization, soluble human IL-4Rα was immobilized by coating the plates with 2 μg/ml of IL-4Rα in phosphate buffered saline (PBS) overnight at 4° C. The plates were blocked with 200 μL of a 0.5% solution of bovine serum albumin (Sigma, St. Louis, Mo.) diluted in PBS prior to adding antibody/IL-4. After addition of the antibody/IL-4 mixture, the plates were incubated for 60 minutes at room temperature. Following the incubation, the plates were washed 3 times with PBS containing 0.05% Polysorbate 20 (Sigma). Horseradish peroxidase conjugated to streptavidin (Jackson ImmunoResearch, West Grove, Pa.) was diluted 1:5000 in the assay buffer and 100 μL was added to each well. Following a 30 minute incubation at room temperature, the plates were washed as described above. 100 μL of the TMB substrate was added and the plate was incubated for 5 to 15 minutes. Reactions were stopped by the addition of IN Phosphoric Acid. The ELISA plates were read at OD450 using a Spectra Max 340 plate reader (Molecular Devices, Sunnyvale, Calif. Curves were plotted using Kaleidagraph graphing software (Synergy Software, Reading, Pa.).

To determine whether 19C11 blocks IL-4-induced proliferation of TF-1 cells, serial dilutions of purified 19C11 or irrelevant control antibody were incubated with IL-4 and TF-1 cells. Following a 48 hour incubation, each sample received ³H-thymidine and after a 4 hour incubation incorporation of ³H-thymidine was determined.

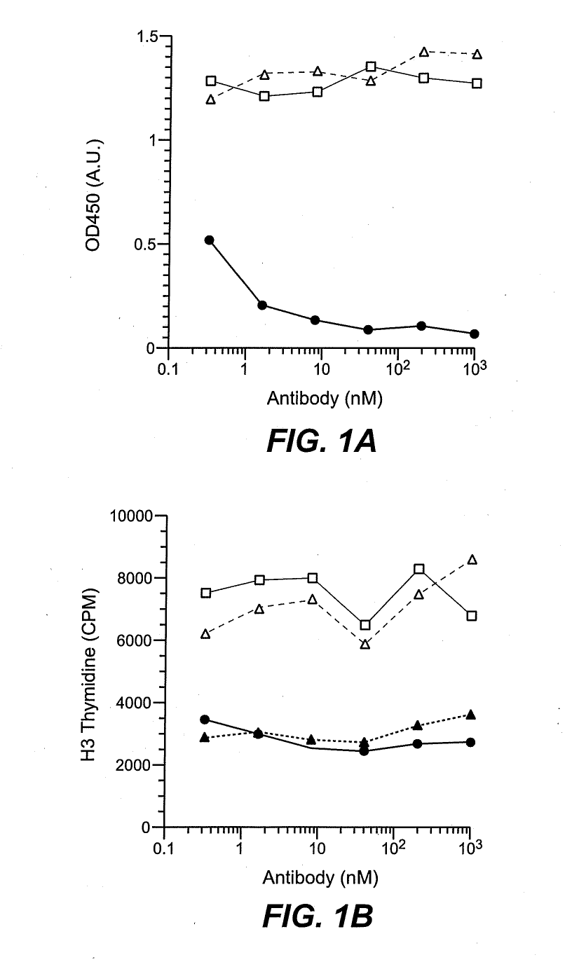

19C11 blocked binding of biotinylated IL-4 to IL-4Rα (FIG. 1A), suggesting an epitope on IL-4 that overlaps with a region involved in binding to IL-4Rα. 19C11 also inhibited IL-4-induced proliferation of TF-1 cells (FIG. 1B). The IC50 for blocking IL-4-induced proliferation of TF-1 cells was determined to be 0.014 μg/ml, and the IC90 was determined to be 0.07 μg/ml (data not shown). 19C11 was subsequently humanized by grafting the hypervariable region into a human Vkappa-1/VHIII acceptor framework with select point mutations. The binding affinity, epitope, and cellular activity of 19C11 were conserved in the humanization process (data not shown).

### Example 3

### Humanization of 19C11

The hypervariable regions (HVRs) from mu19C11 were grafted into the human VL kappa I (huKI), VL kappa VH subgroup I (huVH1) and VH subgroup III (huVHIII) consensus acceptor frameworks to generate CDR grafts (19C11-κ1 graft, 19C11-κ3 graft, 19C11-VH1 graft, 19C11-VH3 graft) (see FIGS. 10 to 13). In the VL domain the following regions were grafted: positions 24-34 (HVRL1, SEQ ID NO: 15), 50-56 (HVRL2, SEQ ID NO: 16) and 89-97 (HVRL3, SEQ ID NO: 17). In the VH domain, positions 26-35b (HVRH1, SEQ ID NO: 12), 49-65 (HVRH2, SEQ ID NO: 13) and 95-102 (HVRH3, SEQ ID NO: 14) were grafted.

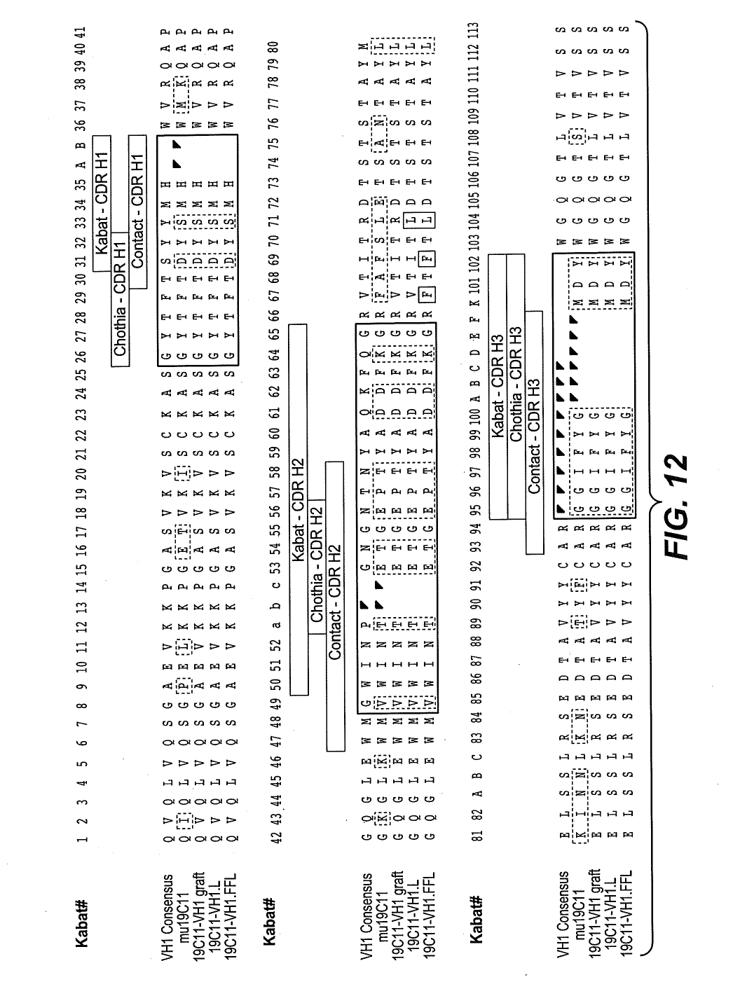

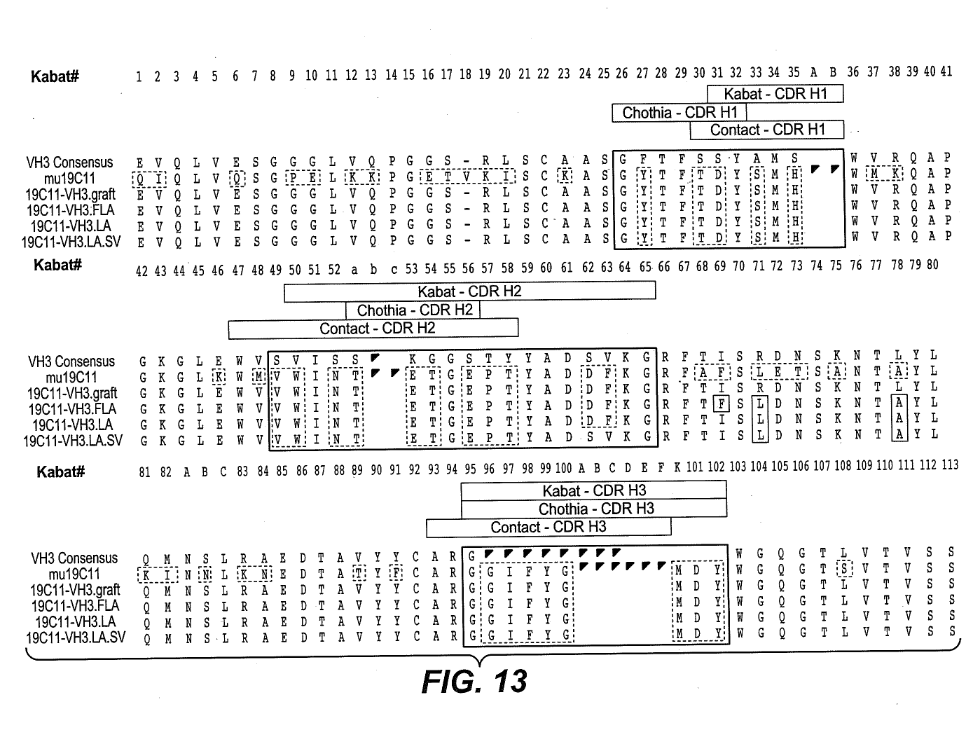

The 19C11-grafts were generated by Kunkel mutagenesis as IgG expression constructs using separate oligonucleotides for each hypervariable region. Correct clones were identified by DNA sequencing. To potentially enhance the affinity and function of the 19C11-grafts, certain murine vernier framework positions were restored in the VH domain grafts (see FIGS. 12 and 13). Specifically, positions 67, 69 and 71 of 19C11-VH1 graft, and positions 69, 71 and 78 of 19C11-VH3 graft were diversified to generate 19C11-VH1.L, 19C11-VH1.FFL, 19C11-VH3.LA, and 19C11-VH3. FLA. In addition, mutations D62S and F63V were introduced into CDR-H2 of 19C11-VH3.LA to generate 19C11-VH3.LA.SV (see FIG. 13).

For screening purposes, IgG variants were initially produced in 293 cells in 6-well plates. Vectors coding for VL and VH (2 μg each) were transfected into 293 cells using the FuGene system. 6 μl of FuGene was mixed with 100 μl of DMEM media containing no FBS and incubated at room temperature for 5 minutes. Each chain (2 μg) was added to this mixture and incubated at room temperature for 20 minutes and then transferred to 6-well plates for transfection overnight at 37° C. in 5% CO₂. The following day the media containing the transfection mixture was removed and replaced with 2 ml cell culture media, e.g., DMEM containing FBS. Cells were incubated for an additional 5 days, after which the media was harvested at 1000 rpm for 5 minutes and sterile filtered using a 0.22 μm low protein-binding filter. Samples are stored at 4° C.

Affinity determinations were performed by surface plasmon resonance using a BIAcore™-A100. Anti-human Fcγ antibody (approximately ˜7000 RU) was immobilized in 10 mM sodium acetate pH 4.8 on a CM5 sensor chip. Humanized 19C11 IgG variants expressed in 293 cells were captured by anti-human Fcγ antibody. Recombinant IL-4 was then injected at a flow rate of 30 μL/min. After each injection the chip was regenerated using 3 M MgCL₂. Binding response was corrected by subtracting a control flow cell from humanized 19C11 variant IgG flow cells. A 1:1 Languir model of simultaneous fitting of kₒₙ and k_{off} was used for kinetics analysis.

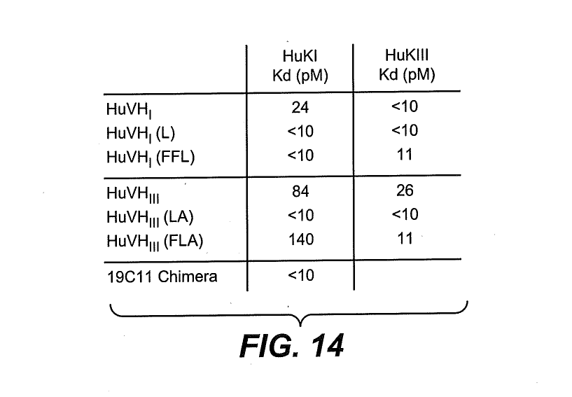

Twelve different humanized 19C11 variants were made, combining each of the humanized light chains (19C11-κ1 graft, 19C11-κ3 graft) with each of the humanized heavy chains (19C11-VH1 graft, 19C11-VH1.L, 19C11-VH1.FFL, 19C11-VH3 graft, 19C11-VH3.LA, and 19C11-VH3. FLA). The twelve humanized 19C11 variants were tested for IL-4 affinity by SPR, along with a chimeric 19C11 in which the mouse variable regions were combined with human IgG constant regions (FIG. 14). Most of the variants retained an affinity for IL-4 of less than 10 pM, with the exception of 19C11-VH1 graft/κ1 graft, 19C11-VH3 graft/κ1 graft, 19C11-VH3.FLA/κ1 graft, and 19C11-VH3 graft/κ3 graft. 19C11-VH1.FFL/κ3 graft and 19C11-VH3.FLA/κ3 graft had an affinity for IL-4 of 11 pM.

19C11-VH3.LA.SV/κ1 graft was selected for further study. The heavy chain and light chain variable region sequences for humanized antibody 19C11-VH3.LA.SV/κ1 graft (referred to in the Examples below as anti-IL-4) are shown in SEQ ID NOs: 9 and 10, respectively. The heavy chain hypervariable regions (HVRs) for antibody 19C11-VH3.LA.SV/κ1 graft are shown in SEQ ID NOs: 12 to 14, and the light chain HVRs are shown in SEQ ID NOs: 15 to 17.

### Example 4

### Generation of IL-4/IL-13 IgG1 Bispecific Antibody

We previously established a technology to generate human IgG1 bispecific antibodies with two different light chains in E. coli (Yu et al., 2011, Sci Transl Med 3, 84ra44). The method utilizes knobs-into-holes technology (Ridgway et al., 1996, Protein Eng. 9, 617-621; Atwell et al., 1997, J Mol Biot 270, 26-35) to promote hetero-dimerization of immunoglobulin heavy chains. To enable the use of two different light chains without light chain mispairing, we cultured each arm as a hemimer in separate E. coli cells. We applied this approach to generate the anti-IL-4/IL-13 bispecific antibody by subcloning the anti-IL-4 and anti-IL-13 parental antibodies into vectors allowing the expression of the anti-IL-4 arm as a human IgG1 hole and of the anti-IL-13 arm as a human IgG1 knob. The sequence of the IgG1 knob constant region is shown in SEQ ID NO: 34 and the sequence of the IgG1 hole constant region is shown in SEQ ID NO: 35.

We based the anti-IL-13 Fab of the bispecific antibody on lebrikizumab, which has been previously generated and characterized. See, e.g., PCT Publication No. WO 2005/062967 A2. Lebrikizumab binds soluble human IL-13 with a Biacore-derived Kd that is lower than the detection limit of 10 pM. Binding of lebrikizumab to IL-13 does not inhibit binding of the cytokine to IL-13Rα1, but does block the subsequent formation of the heterodimeric signaling competent IL-4Rα/IL-13Rα1 complex (Ultsch, M. et al., 2013, J. Mol. Biol., dx.doi.org/10.1016/j.jmb.0.2013.01.024; Corren et al., 2011, N. Engl. J. Med. 365, 1088-1098).

For antibody expression, E. coli strain 64B4 was used. An overnight culture was grown at 30° C. in LB (100 μg/ml carbenicillin), diluted 1:100 into 5 ml CRAP media (100 μg/ml carbenicillin) (Simmons et al., 2002, J. Immunol. Methods, 263: 133-147) and grown for 24 hours at 30° C. After expression, the soluble fractions were subjected to SDS-PAGE followed by anti-Fc immunostaining to analyze the formation of half-antibody species. The knob and hole mutations both result in a predominant half-antibody species. For scale-up to 10 L fermenters, initial starter cultures (500 ml) were grown into stationary phase and used to inoculate 10 L fermentations (Simmons et al., 2002, J. Immunol. Methods, 263: 133-147).

Initial expression of anti-IL-13 IgG1 knob hemimer in E. coli was lower than expected. It has previously been shown that random mutagenesis and/or replacing hydrophobic surface residues of a Fab sequence can lead to improved Fab stability and folding (Forsberg et al., 1997, J. Biol. Chem., 272: 12430-12436; Demarest et al., 2006, Protein Eng. Des. Sel., 19: 325-336; Kugler et al., 2009, Protein Eng. Des. Sel., 22: 135-147).

Variants were expressed in E. coli cells, and non-reducing whole cell extracts were analyzed by non-reducing SDS-PAGE followed by anti-Fc immunoblot. The hemimer band was quantified using an Odyssey® (LiCOR Biosciences) and normalized to the lebrikizumab signal.

Several changes in the heavy chain and light chain were found to improve hemimer yield and/or folding. One of the changes, M4L in the light chain, was selected. In addition, a Q1E change was introduced in the heavy chain. The two changes were combined in a single hemimer, and the resulting hemimer was found to have improved yield and folding over the wild-type hemimer. The sequence of the lebrikizumab Q1E heavy chain variable region is shown in SEQ ID NO: 19 and the sequence of the lebrikizumab M4L light chain variable region is shown in SEQ ID NO: 20. Those variable regions were used to construct the anti-IL-4/IL-13 IgG1 bispecific antibody.

The intact bispecific antibody was assembled from isolated half-antibodies by redox-chemistry using methods previously described, for example, in U.S. Patent Publication No. 2011/0287009 and International Patent Application No. PCT/US2012/059810.

### Example 5

### Generation of IL-4/IL-13 IgG4 Bispecific Antibody

After establishing the production of an anti-IL-4/IL-13 bispecific antibody of human IgG1 isotype, we changed the bispecific platform to the human IgG4 isotype. We wished to make the anti-IL-4/IL-13 bispecific antibody as a human IgG4 antibody in order to match the isotype of lebrikizumab, the anti-IL-13 antibody which has shown clinical benefit in the treatment of moderate-to-severe uncontrolled asthma (Corren et al., 2011, N. Engl. J. Med. 365, 1088-1098).

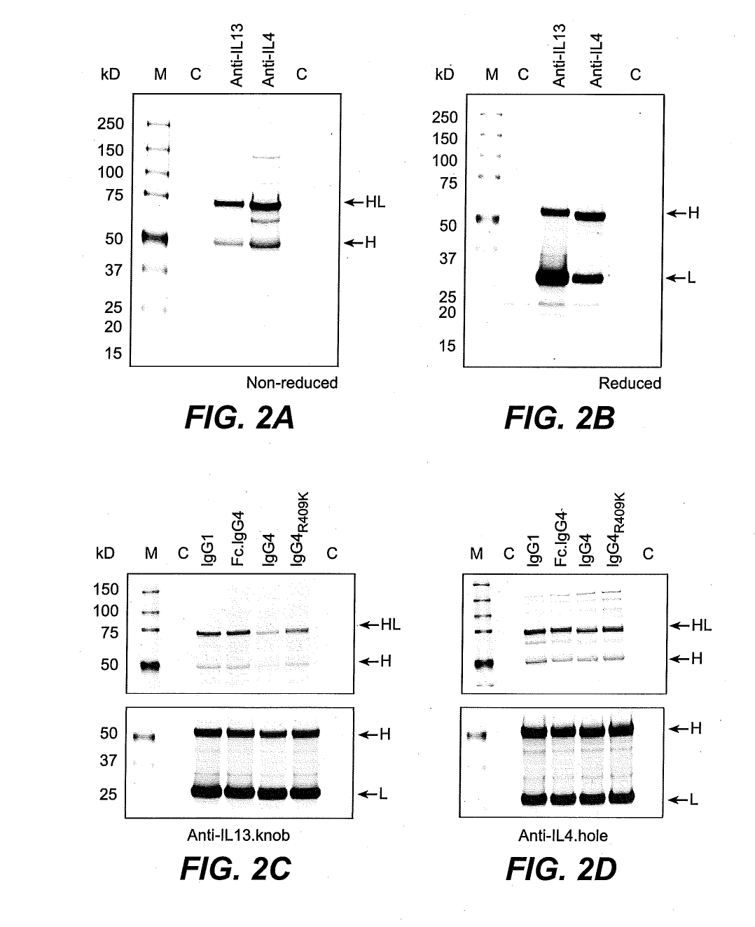

In contrast to IgG1, the heavy-light interchain disulfide of IgG4 is formed by non-consecutive disulfides. This non-consecutive disulfide linkage-pattern is not commonly observed for E. coli proteins (Berkmen, 2005, J. Biol. Chem. 280, 11387-11394). In addition, the hinge region of IgG4 is destabilized by an S228 residue, and the CH3 dimer interface of IgG4 contains a destabilizing R409 residue (Dall'Acqua et al., 1998, Biochemistry 37, 9266-9273) (EU numbering convention). We designed several constructs to dissect the impact of the IgG4 Fc region sequence, the inter-chain disulfide pattern, and the CH3 R409 on the functional expression of the half-antibodies in E. coli and subsequent assembly to a bispecific molecule. In each case, we introduced a stabilizing S228P mutation in the hinge region to attenuate Fab arm exchange after assembly (Stubenrauch et al., 2010, Drug Metab. Dispos. 38, 84-91). We first grafted the IgG4 Fc region with corresponding knob/hole mutations (knob: T366W; hole: T366S, L368A, Y407V) onto the IgG1 Fab in order to assess the impact of the IgG4 Fc region on functional expression of the half-antibody. For both antibodies, anti-IL-4 and anti-IL-13, this yielded similar amounts of disulfide-bonded material as the IgG1 isotype (FIGS. 2C and 2D), indicating that the differences between the isotypes in the Fc region do not impact functional half-antibody expression in E. coli. We next converted the entire constant region of the heavy chain to the IgG4 subclass. While this resulted in a reduction in functionally expressed half-antibody, it demonstrated that E. coli is in principle capable of forming intramolecular disulfides in the constant region of the antibody from non-consecutive cysteines.

Since position 409 may be important for the CH3 stability (Dall'Acqua et al., 1998, Biochemistry 37, 9266-9273) and the impact of R409 for a downstream assembly process was uncertain at this stage, we also designed a construct with an R409K mutation, to recreate the CH3 interface found in the IgG1 isotype. For both antibodies, this partially rescued the slight drop in functional expression of the IgG4 isotype (FIGS. 2C and 2D).

### Example 6

### Assembly and Purification of IL-4/IL-13 Bispecific Antibodies

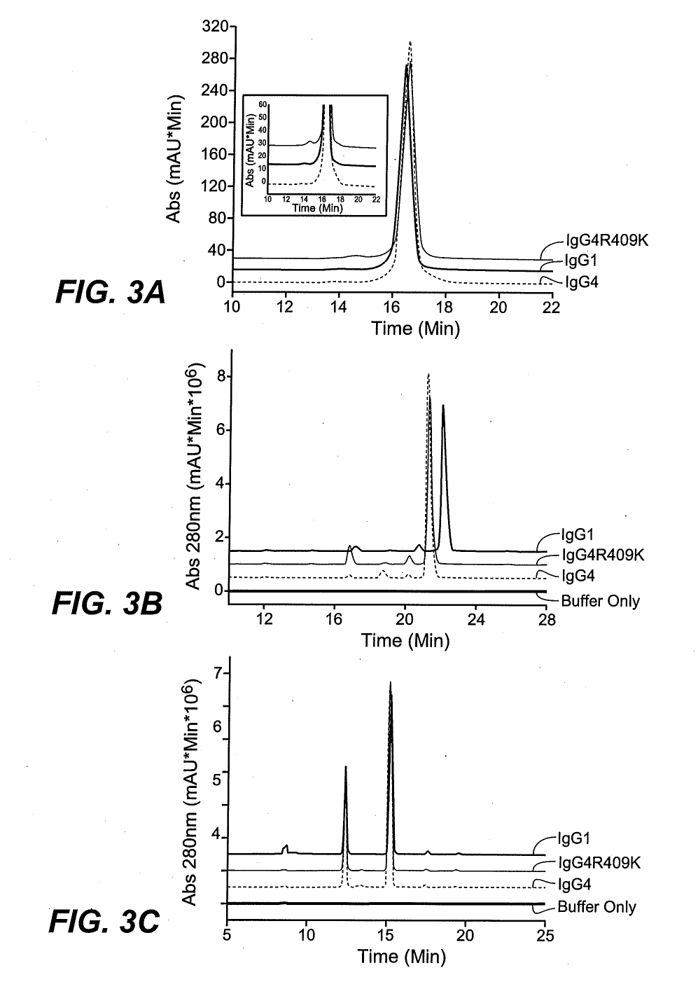

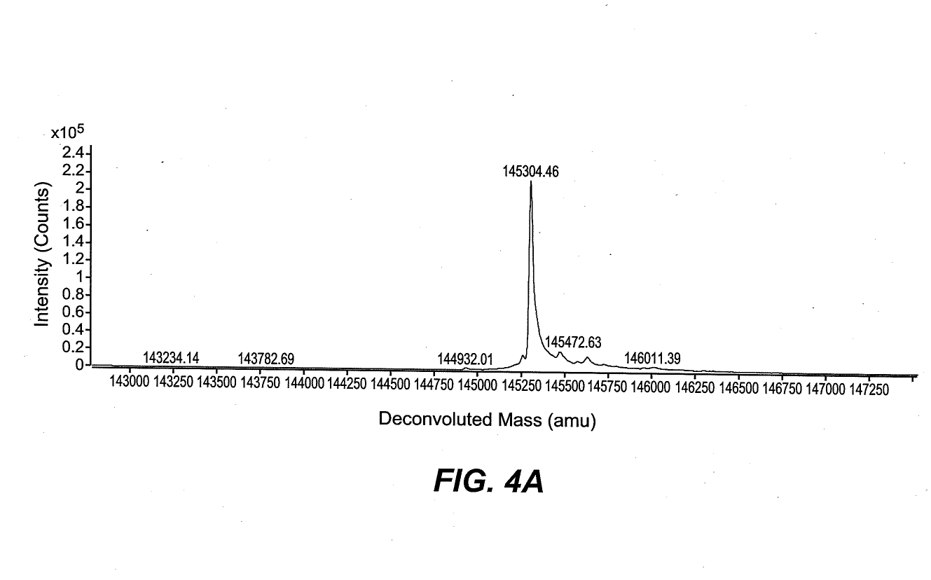

To compare the assembly of the different bispecific antibody constructs, we grew cultures expressing half-antibodies as IgG1, IgG4 and IgG4R409K. After purification of the half-antibodies by Protein A chromatography, the hemimer pairs were mixed, and the intact bispecific antibody formed by a redox chemistry step of the heterodimerized knob/hole pairs. Excess half-antibody was removed by anion and cation-exchange chromatography steps. After the final chromatography step the material was formulated at 45 g/l in 0.2 M Arginine Succinate pH 5.5, 0.02% Polysorbate-20. To confirm that the assembled antibodies shifted from the half-antibody species to a stable intact antibody, we characterized them by size exclusion chromatography. All three constructs eluted with a retention time corresponding to an intact, 150 kDa antibody (FIG. 3A). Furthermore no significant amounts of aggregated species (0.6/0.4/0.4% for IgG1/IgG4/IgG4R409K) and only trace amounts of low molecular weight species (0.2/0/4.4% for IgG1/IgG4/IgG4R409K) were detected, suggesting that both isotypes can be used to assemble antibodies of low aggregation propensity.

One of the steps during bispecific assembly is the formation of the hinge-disulfides. Since size exclusion chromatography cannot resolve the oxidation state of the interchain disulfides, we subjected the antibodies to capillary electrophoresis-sodium dodecyl sulfate analysis (CE-SDS) and found that all three formats formed hinge-disulfides with similar efficiency. For IgG1, IgG4 and IgG4R409K, 89.3%, 91.4%, and 86.7% of the material was observed in the fully-oxidized conformation, respectively (FIG. 3B). We next reduced the samples and reanalyzed them by CE-SDS to determine the respective ratios of light to heavy chains (FIG. 3C). All three formats had a similar and expected distribution of light (31.3/31.4/30.9% for IgG1/IgG4/IgG4R409K) and heavy chains (65.8/64.9/65.4% for IgG1/IgG4/IgG4R409K), further confirming the existence of a natural antibody conformation.

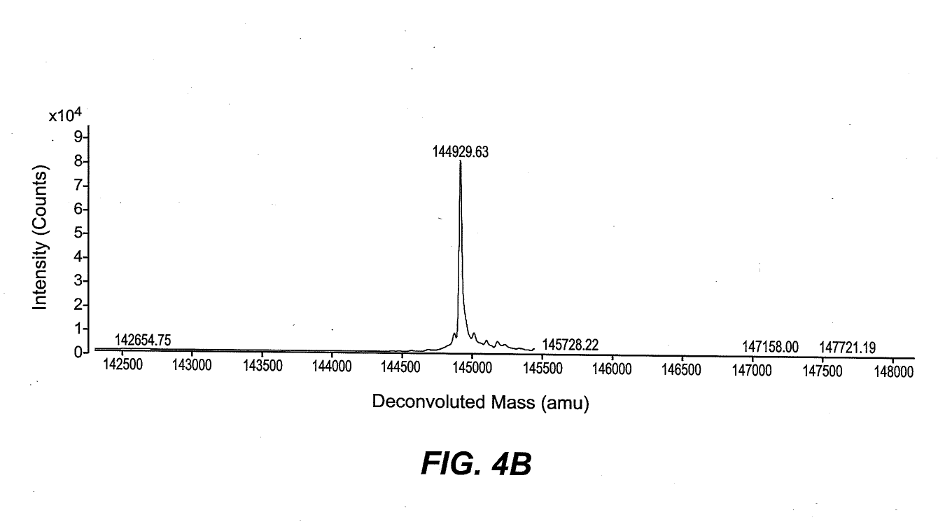

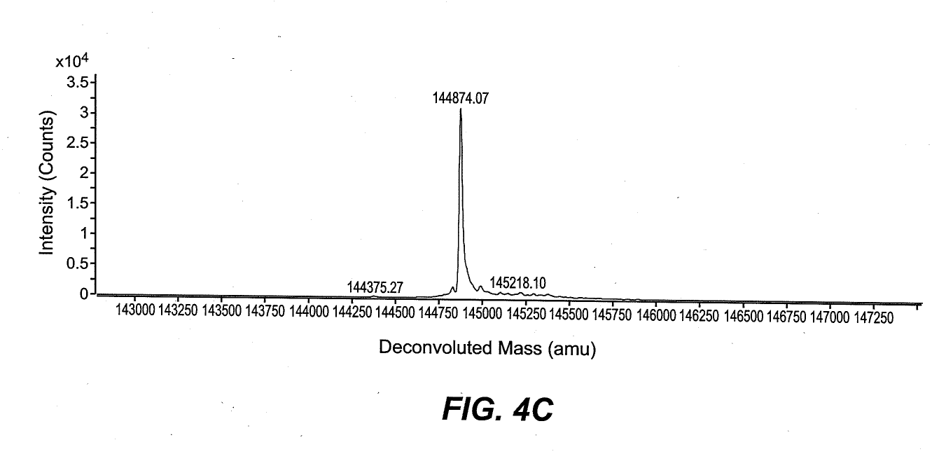

To ensure that heterodimeric species were generated during the assembly process, we analyzed the final bispecific molecules by mass spectrometry. The intact and reduced masses are summarized in Table 2, FIG. 4 and Table 3. For all three bispecific antibodies, the experimental masses matched closely the theoretical masses, and we were not able to detect any masses corresponding to homodimeric species. A reverse-phase HPLC assay further confirmed that the antibodies were bispecific, with no evidence of homodimeric antibodies (data not shown).

TABLE 2
Mass Spectrometric Analysis of non-reduced
anti-IL-4/IL-13 Bispecific Antibodies

| | Theoretical | Experimental |
|---|---|---|
| | Mass | Mass |
| | (Da) | (Da) |
| anti-IL-4/IL-13 IgG1 Bispecific | 145298.4 | 145304.5 |
| anti-IL-4 IgG1 homodimer | 144798.6 | n.o. |
| anti-IL-13 IgG1 homodimer | 145798.3 | n.o. |
| anti-IL-4/IL-13 IgG4 Bispecific | 144923.7 | 144929.6 |
| anti-IL-4 IgG4 homodimer | 144423.9 | n.o. |
| anti-IL-13 IgG4 homodimer | 145423.5 | n.o. |
| anti-IL-4/IL-13 IgG4_{R409K} Bispecific | 144867.7 | 144874.0 |
| anti-IL-4 IgG4_{R409K} homodimer | 144367.8 | n.o. |
| anti-IL-13 IgG4_{R409K} homodimer | 145367.5 | n.o. |

n.o. not observed

TABLE 3
Mass Spectrometric Analysis of reduced
anti-IL-4/IL-13 Bispecific Antibodies

| | Theoretical | Experimental |
|---|---|---|
| | Mass | Mass |
| | (Da) | (Da) |
| anti-IL-4 LC IgG1 | 23522 | 23521 |
| anti-IL-4 HC IgG1 | 48893 | 48893 |
| anti-IL-13 LC IgG1 | 23815 | 23815 |
| anti-IL-13 HC IgG1 | 49100 | 49099 |
| anti-IL-4 LC IgG4 | 23522 | 23523 |
| anti-IL-4 HC IgG4 | 48706 | 48708 |
| anti-IL-13 LC IgG4 | 23815 | 23816 |
| anti-IL-13 HC IgG4 | 48913 | 48914 |
| anti-IL-4 LC IgG4_{R409K} | 23522 | 23523 |
| anti-IL-4 HC IgG4_{R409K} | 48678 | 48679 |
| anti-IL-13 LC IgG4_{R409K} | 23815 | 23816 |
| anti-IL-13 HC IgG4_{R409K} | 48885 | 48886 |

LC light chain,
HC heavy chain

Since we could not detect any significant differences in the assembly of R409 and R409K IgG4 bispecific knobs-into-holes antibodies, all further studies utilized the wildtype (R409) IgG4 bispecific antibody format.

### Example 7

### Biochemical Characterization of IL-4/IL-13 Bispecific Antibodies

We next characterized the IgG1 and IgG4 bispecific antibodies to assess whether their binding affinities to IL-4 and IL-13, as well as their ability to block the binding of IL-4 and IL-13 to their receptors, were comparable. The affinities of the IgG1 and IgG4 bispecific antibodies for IL-4 and IL-13 were measured by Biacore as described in Example 1 and were found to be comparable (Table 4) and similar to those of the parental antibodies, indicating that the ability to bind ligand is not impacted by the bispecific format or the isotype.

Anti-IL-4/IL-13 bispecific antibody binds with high affinity to human IL-13, human IL-13 R130Q (SEQ ID NO: 31), and cyno IL-13. Dissociation constants of 0.056, 0.142, and 0.048 (nM) were calculated for those cytokines, respectively. Kinetic constants are provided in Table 4. Additional SPR experiments showed the anti-IL-4/IL-13 bispecific antibody binds with high affinity to human IL-4 and cyno IL-4. Dissociation constants of 0.046 and 0.076 nM were calculated for those cytokines, respectively. Kinetic constants are provided in Table 4.

TABLE 4
Binding kinetics of anti-IL-4/IL-13 bispecific antibodies

| Iso- | | Kₒₙ/10⁴ | k_{off}/10⁻⁴ | K_{d} |
|---|---|---|---|---|
| type | Ligand | (M⁻¹s⁻¹) | (s⁻¹) | (nM) |
| IgG1 | human IL-4 | 134.4 ± 49.8 | 0.848 ± 0.05 | 0.068 ± 0.020 |
| IgG4 | human IL-4 | 287.00 ± 4.58 | 1.327 ± 0.058 | 0.046 ± 0.001 |
| IgG1 | human IL-13 | 71.4 ± 4.0 | 0.170 ± 0.119 | 0.023 ± 0.015 |
| IgG4 | human IL-13 | 53.73 ± 2.1 | 0.301 ± 0.109 | 0.056 ± 0.020 |
| IgG4 | human IL-13 | 1.84 ± 0.13 | 0.262 ± 0.036 | 0.142 ± 0.013 |
| | R130Q | | | |
| IgG4 | cyno IL-4 | 201.67 ± 39.15 | 1.507 ± 0.153 | 0.076 ± 0.013 |
| IgG4 | cyno IL-13 | 60.80 ± 4.94 | 0.283 ± 0.202 | 0.048 ± 0.036 |

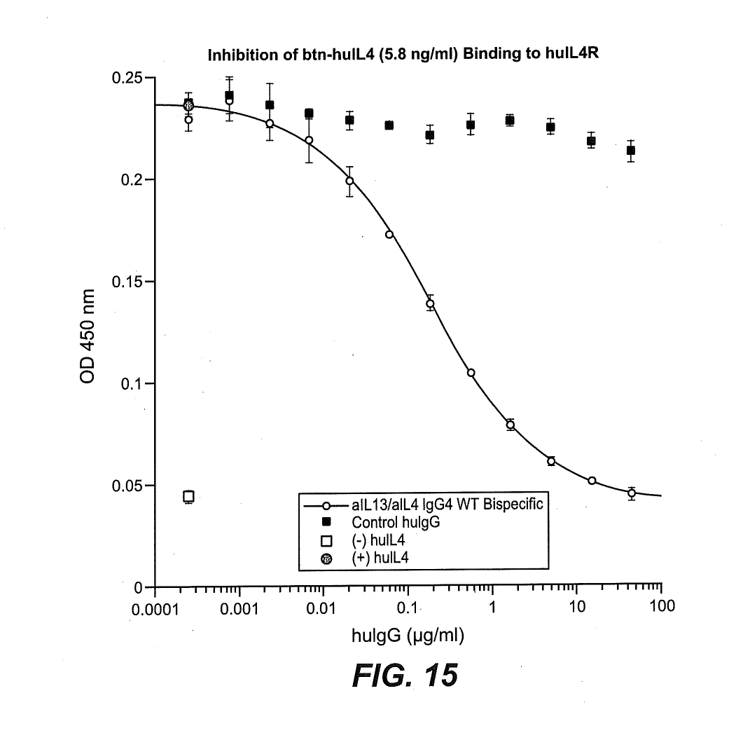

To ensure that the bispecific molecule can block binding of cytokine to its receptor, ELISA binding competition assays substantially as described in Example 1 were used. Anti-IL-4/IL-13 bispecific antibody inhibited biotinylated human IL-4 (5.8 ng/mL) direct binding to human IL-4R (see FIG. 15). A decrease in biotinylated IL-4 binding to IL-4R was observed at 0.035 to 25 μg/mL (0.23 to 167 nM) of bispecific antibody.

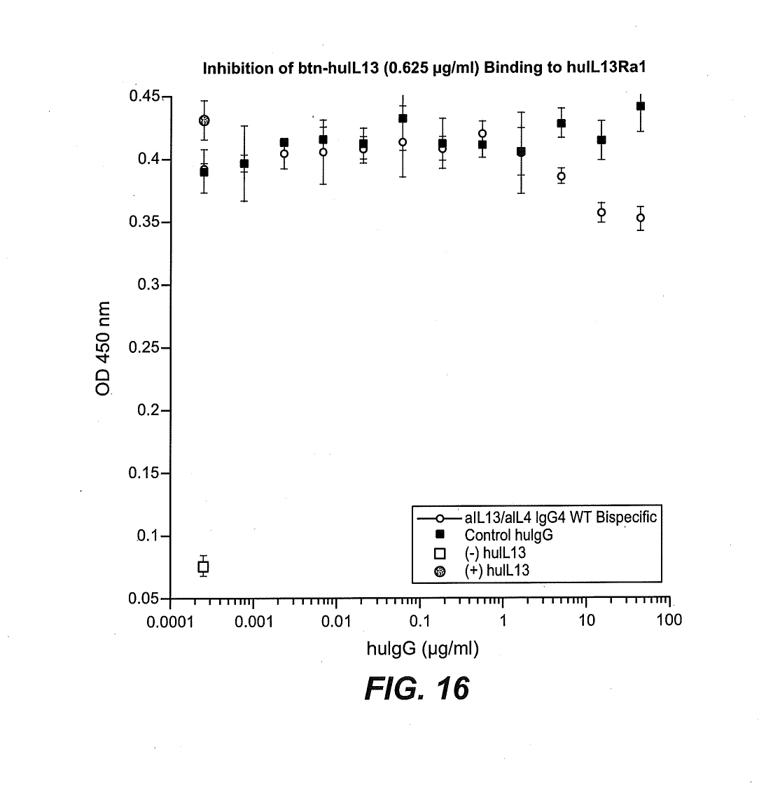

In contrast, anti-IL-4/IL-13 bispecific antibody did not inhibit biotinylated human IL-13 (0.625 μg/mL) direct binding to human IL-13Rα1 (see FIG. 16). No decrease in biotinylated human IL-13 binding to IL-13Rα1 was observed with the addition of bispecific antibody at the concentrations tested.

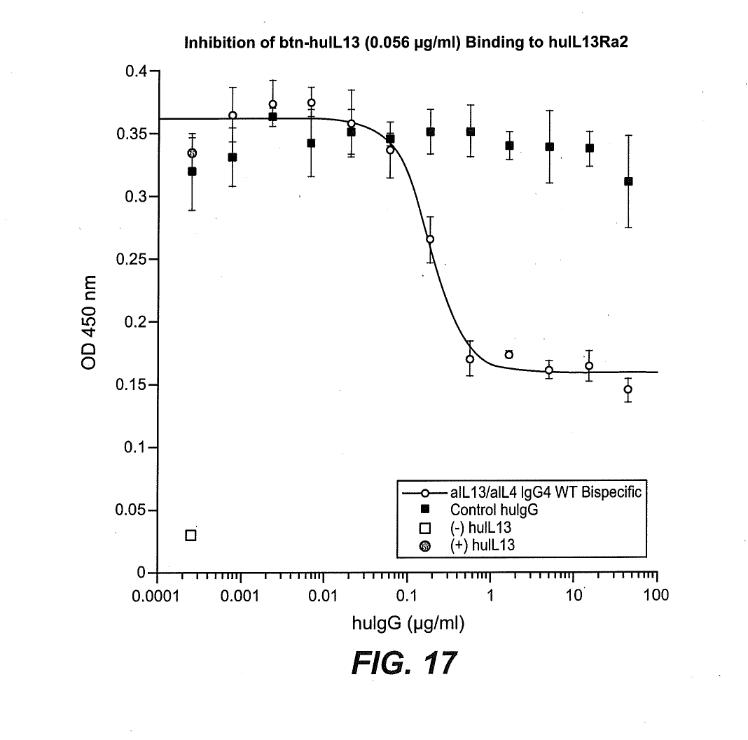

Anti-IL-4/IL-13 bispecific did not substantially inhibit biotinylated human IL-13 (0.056 μg/mL) direct binding to human IL-13Rα2 (see FIG. 17). A partial decrease in biotinylated IL-13 binding to IL-13Rα2 was observed.

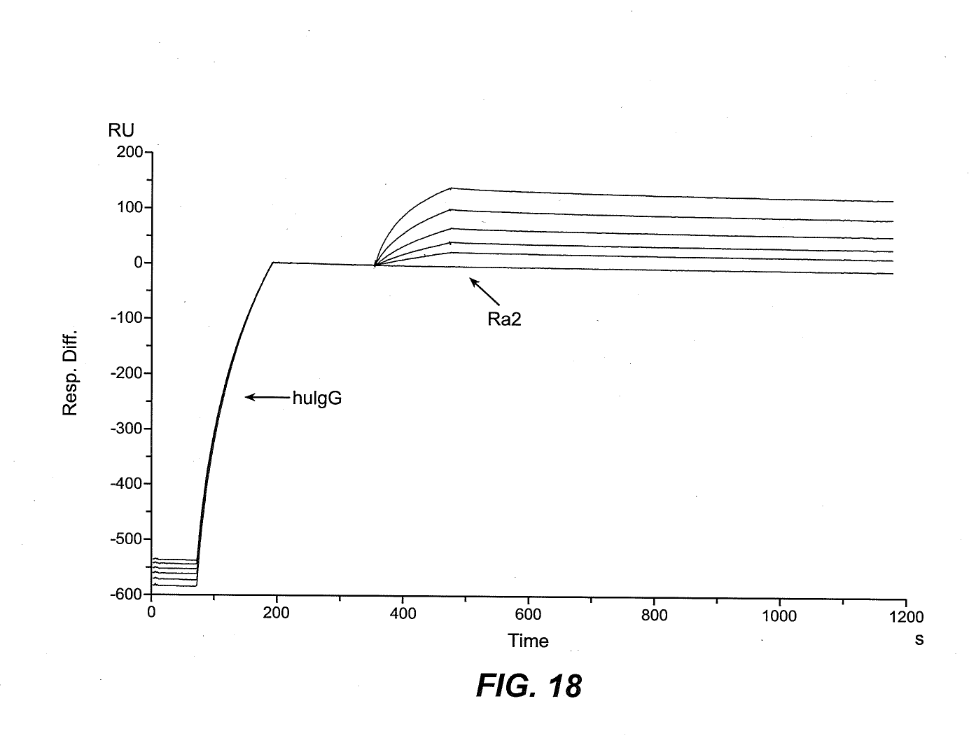

SPR was used to observe the binding of IL-13Rα2 to IL-13 as described in Example 1. Sensograms were collected for injection of a series of concentrations of IL-13Rα2 over immobilized IL-13. Based on the sensograms, a binding constant (Kd) of 0.365 nM (kₒₙ=24.27×10⁴±0.49 Ms⁻¹, k_{off}=0.891×10⁻⁴±0.026 s⁻¹) was observed. Anti-IL-4/IL-13 bispecific antibody was previously shown to bind IL-13 with high affinity (Kd=56 pM) in separate SPR experiments (see Table 4). To test the inhibition of IL-13Rα2 binding to IL-13, 250 nM anti-IL-4/IL-13 bispecific antibody was injected over the immobilized IL-13 prior to injection of IL-13Rα2. Binding of the bispecific antibody did not prevent association of IL-13Rα2 with the immobilized IL-13 (see FIG. 18). The Kd for binding of IL-13Rα2 to the bispecific antibody:IL-13 complex was 1.09 nM (kₒₙ=10.06×10⁴±0.56 Ms⁻¹, k_{off}=1.10×10⁻⁴+0.12 s⁻¹). Presaturation of immobilized IL-13 with bispecific antibody only modestly disrupted IL-13Rα2 binding to IL-13 and indicates that the bispecific antibody does not significantly inhibit IL-13 binding to IL-13Rα2.

Thus, similar to the parental anti-IL-4 and anti-IL-13 antibodies, the bispecific antibody fully inhibited binding of IL-4 to IL-4Rα, and did not substantially inhibit binding of IL-13 to IL-13Rα1 or IL-13Rα2. These findings suggest that the binding epitope and monovalent affinity for each IL-13 and IL-4 arm was conserved in the bispecific antibodies.

### Example 8

### Neutralization of IL-4 and IL-13 Activity in a Cellular Assay

The activity of both anti-IL-4/IL-13 IgG1 and anti-IL-4/IL-13 IgG4 bispecific antibodies was assessed in an in vitro cellular assay in which human IL-4 and IL-13 induce the proliferation of TF-1 cells. The ability of each bispecific antibody to block proliferation of TF-1 cells induced by human IL-4 and human IL-13 alone and in combination was evaluated as described below.

Antibodies were serially diluted 3.3 fold in 50 μl of assay media containing cytokines in a 96 well tissue culture plate (Catalog No. 353072, Falcon BD, Franklin Lakes, N.J.). Plates were incubated for 30 minutes at 37° C. TF-1 cells were washed twice in assay media and resuspended at a final volume of 2.5×10⁵ cells/ml. 50 μl of cells were added to each well for a total volume of 100 μl. Plates were incubated for 4 days in a humidified incubator at 37° C. with 5% CO₂, before the addition of 1 μCi of ³H thymidine per well. After an additional 4 hour incubation, proliferation was measured by cell-associated ³H thymidine incorporation using a liquid scintillation counter. Results from duplicate samples are expressed as mean values. Graphs were generated using KaleidaGraph (Synergy Software, Reading, Pa.).

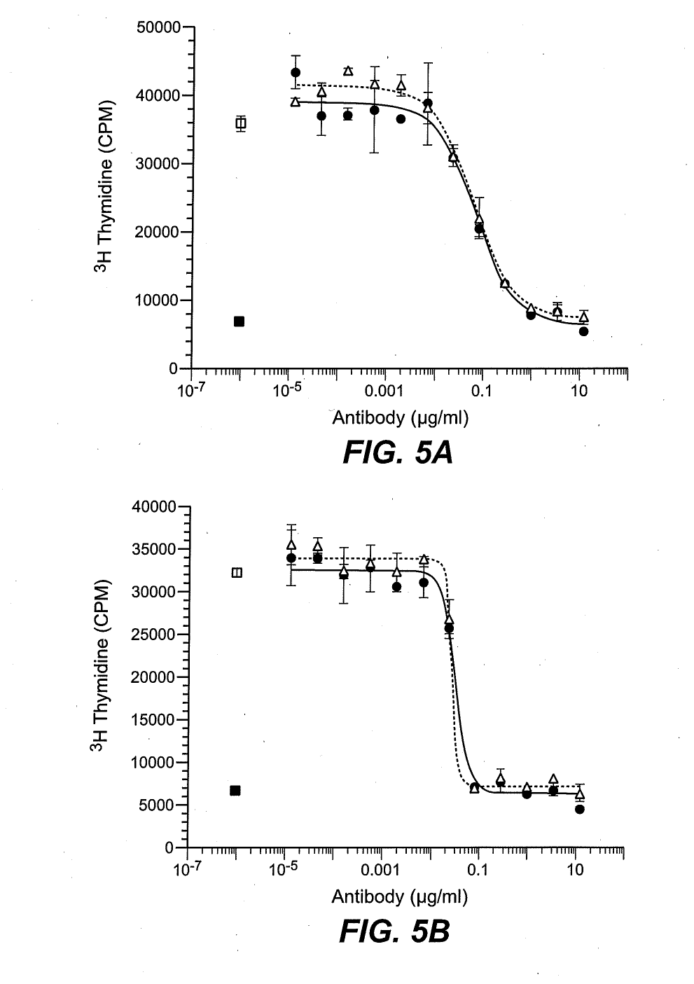

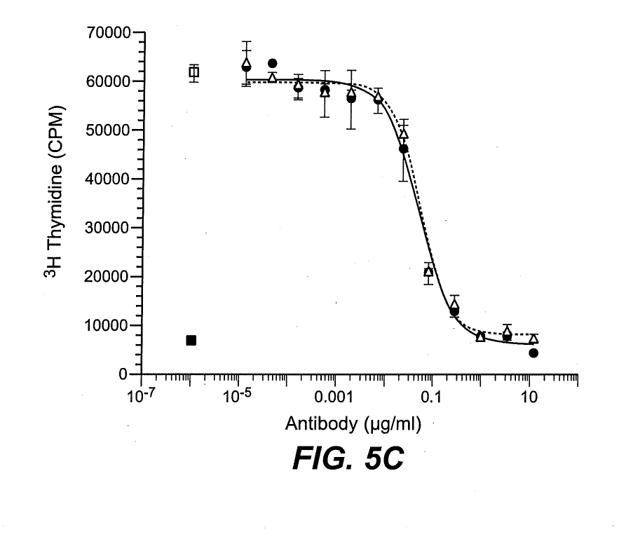

Both anti-IL-4/IL-13 IgG1 and anti-IL-4/IL-13 IgG4 bispecific antibodies inhibited human IL-4- and IL-13-induced proliferation of TF-1 cells in a dose-dependent manner, with no significant differences in the IC₅₀ for in vitro neutralization between the two different bispecific antibodies (FIG. 5 and Table 5).

TABLE 5
IC₅₀ of TF-1 proliferation inhibition assays
for anti-IL-4/IL-13 bispecific antibodies

| | IC₅₀ (μg/ml) | | |
|---|---|---|---|
| | IL-4 | IL-13 | IL-4 + IL-13 |
| IgG1 Bispecific | 0.06 | 0.03 | 0.07 |
| IgG4 Bispecific | 0.05 | 0.03 | 0.05 |

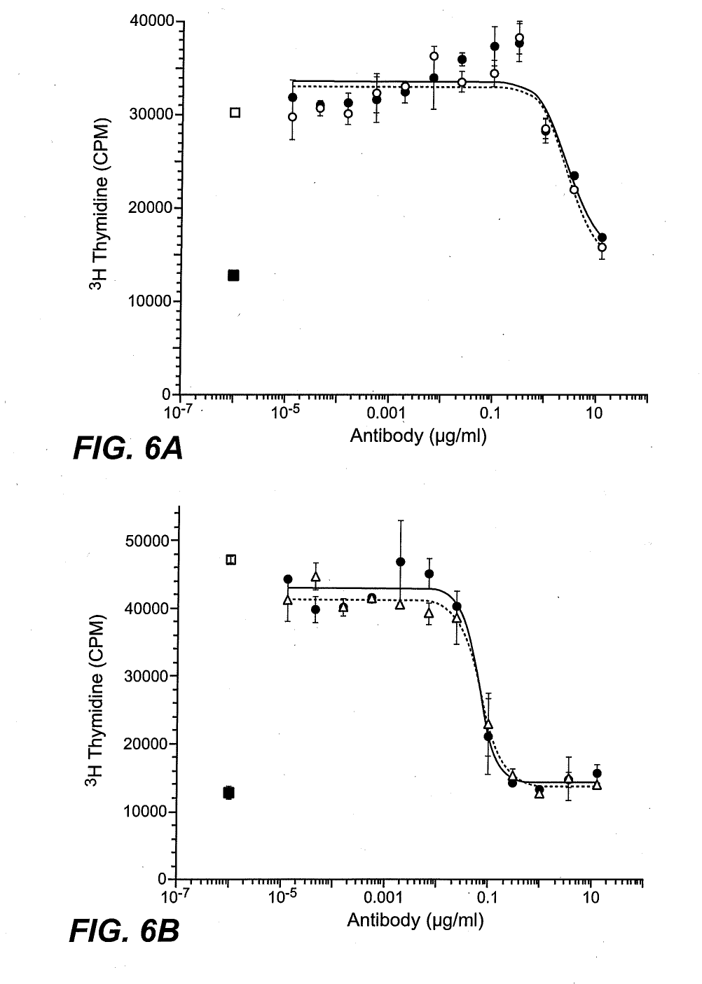

A similar analysis was carried out to determine if anti-IL-4/IL-13 IgG1 and anti-IL-4/IL-13 IgG4 bispecific antibodies inhibited cynomolgus monkey IL-4- and IL-13-induced proliferation of TF-1 cells in a dose-dependent manner (FIG. 6).

### Example 9

### Pharmacokinetic Studies in Cynomolgus Monkeys

We assessed the in vivo pharmacokinetics of the IgG4 and IgG1 anti-IL-4/IL-13 bispecific antibodies following single intravenous (IV) or subcutaneous (SC) administration to cynomolgus monkeys. The pharmacokinetic (PK) studies in cynomolgus monkeys were approved by the Institutional Animal Care and Use Committee (IACUC). The PK study with anti-IL-4/IL-13 IgG4 was conducted at Charles River Laboratories (CRL) Preclinical Services (Reno, Nev.). A total of 15 female cynomolgus monkeys (2.2-2.6 kg) from CRL stock were randomly assigned to five groups (n=3/group). Animals in group 1 were given an intravenous (IV) and subcutaneous (SC) dose of the control vehicle. Animals in groups 2, 3, and 4 were given a single IV bolus dose of anti-IL-4/IL-13 IgG4 at 10, 30, and 100 mg/kg, respectively. Animals in group 5 were given a SC dose of anti-IL-4/IL-13 IgG4 at 10 mg/kg.

The PK study with anti-IL-4/IL-13 IgG1 was conducted at Shin Nippon Biomedical Laboratories (SNBL) USA (Everett, Wash.). A total of 12 female cynomolgus monkeys (2.4-3.1 kg) from SNBL stock were randomly assigned to four groups (n=3/group). Animals in group 1 were given an IV dose of the control vehicle. Animals in groups 2, 3, and 4 were given a single IV bolus dose of anti-IL-4/IL-13 IgG1 at 10, 30, and 60 mg/kg, respectively.

For both studies, serum samples were collected at various time points out to 4-5 weeks post dose and concentrations of anti-IL-4/IL-13 IgG4 or anti-IL-4/IL-13 IgG1 and were assessed by ELISA with limit of quantitation of 0.078 μg/mL and anti-therapeutic antibodies (ATA) by bridging ELISA. For PK data calculations, Study Day 1 was converted to PK Day 0 to indicate the start of dose administration. All time points after the in life dosing day are calculated as Study Day minus 1. The serum concentration data for each animal were analyzed using 2 compartment analysis with WinNonlin®, Version 5.2.1 (Pharsight; Mountain View, Calif.).

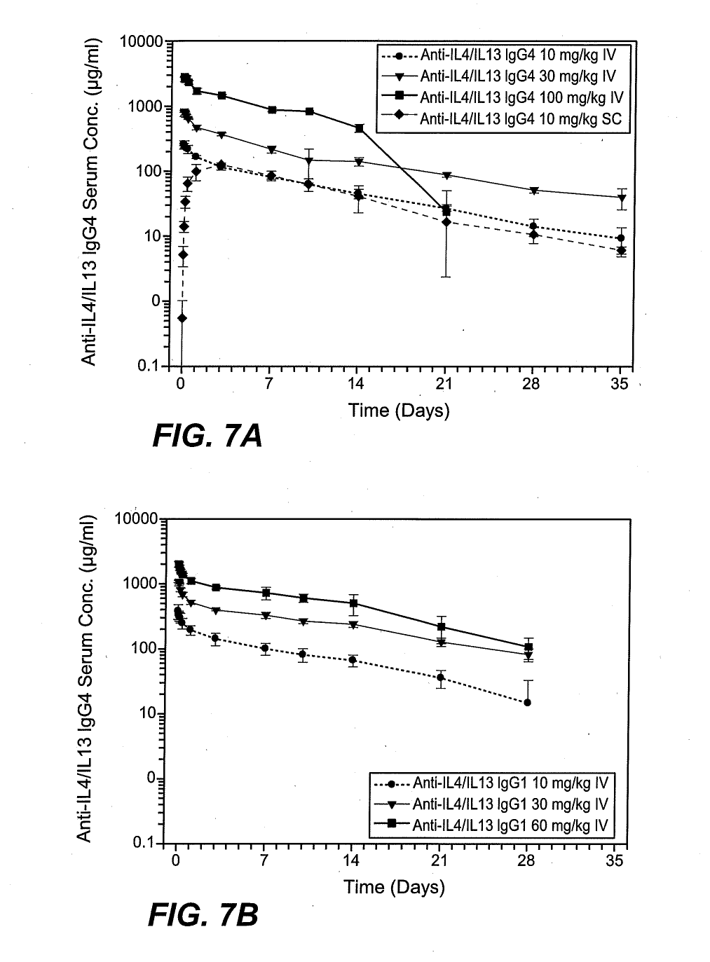

The serum concentration-time profiles of anti-IL-4/IL-13 IgG4 and anti-IL-4/IL-13 IgG1 bispecific antibodies exhibited biphasic disposition with linear pharmacokinetics over the dose range tested (FIGS. 7A and 7B). The initial volume of central compartment for both antibodies was similar to the serum volume indicating limited distribution. Both antibodies had a relatively slow clearance (CL) and a long terminal half-life as expected for human IgG4 and IgG1 antibodies in cynomolgus monkeys (Mean CL=5.79 to 6.70 mL/day/kg for anti-IL-4/IL-13 IgG4 and 3.59 to 4.09 mL/day/kg for anti-IL-4/IL-13 IgG1). Based on the area-under-the curve (AUC) calculated for the 10 mg/kg dose groups, the SC bioavailability of the anti-IL-4/IL-13 IgG4 antibody was 95.1%. The presence of anti-therapeutic antibodies (ATA) was detected in 50% of the anti-IL-4/IL-13 IgG4 dosed animals, including all 3 animals in the 100 mg/kg IV dose group, and appeared to be associated with the increased elimination of anti-IL-4/IL-13 IgG4 after day 14). There was a low incidence of ATA detected in anti-IL-4/IL-13 IgG1 treated animals which did not appear to affect the PK. Overall the pharmacokinetics of both anti-IL-4/IL-13 IgG4 and anti-IL-4/IL-13 IgG1 bispecific antibodies were similar and comparable to that of other humanized IgGland IgG4 monoclonal antibodies in cynomolgus monkeys.

### Example 10

### Lung Partitioning in a Cynomolgus Monkey Asthma Model

We evaluated potential differences in the lung partitioning of IgG4 vs. IgG1 anti-IL-4/IL-13 bispecific antibodies in a cynomolgus monkey model of asthma. In this asthma model, cynomolgus monkeys that were naturally sensitized to Ascaris suum (A. suum) received an aerosol challenge of A. suum extract to elicit allergic inflammatory responses that mimic those of asthmatics exposed to allergens.

The lung partitioning study in cynomolgus monkeys was approved by IACUC. This study comparing anti-IL-4/IL-13 IgG4 and anti-IL-4/IL-13 IgG1 was conducted at CRL, Preclinical Services (Reno, Nev.). The study consisted of two different sessions. In the first session, cynomolgus monkeys (3-10 kg) from CRL stock received a baseline aerosol challenge with Ascaris suum (A. suum) to determine the suitability of the A. suum challenge to elicit appropriate airway responses in each animal. The animals were monitored for signs of distress throughout the challenge period and were not given antibodies during this session. Four weeks later, the second session was initiated and a total of 7 male cynomolgus monkeys were randomly assigned to two groups (n=3 in IgG4 group; n=4 in IgG1 group). These monkeys then received 10 mg/kg of either anti-IL-4/IL-13 IgG4 or anti-IL-4/IL-13 IgG1 via an IV bolus dose on Study Day 1 and Study Day 8. Subsequently, the animals were challenged via aerosol inhalation with A. suum on Study Day 9. At various time points up to 23 days post dose, bronchoalveolar lavage (BAL) fluid and serum samples were collected and analyzed for anti-IL-4/IL-13 IgG4 or anti-IL-4/IL-13 IgG1 concentrations by ELISA with limit of quantitation of 0.078 μg/mL. For data calculations, Study Day 1 was converted to PK Day 0 to indicate the start of dose administration. All time points after the in life dosing day are calculated as Study Day minus 1. Urea and albumin were measured in BAL and serum to estimate epithelial lining fluid (ELF) concentrations and to correct for inflammation induced vascular leakage, respectively. Ascaris specific IgE was also measured in the serum by ELISA. Dilution factors were estimated using BAL and serum urea concentration data as described by Rennard et al., 1986, J. Appl. Physiol., 60(2): 532-538.

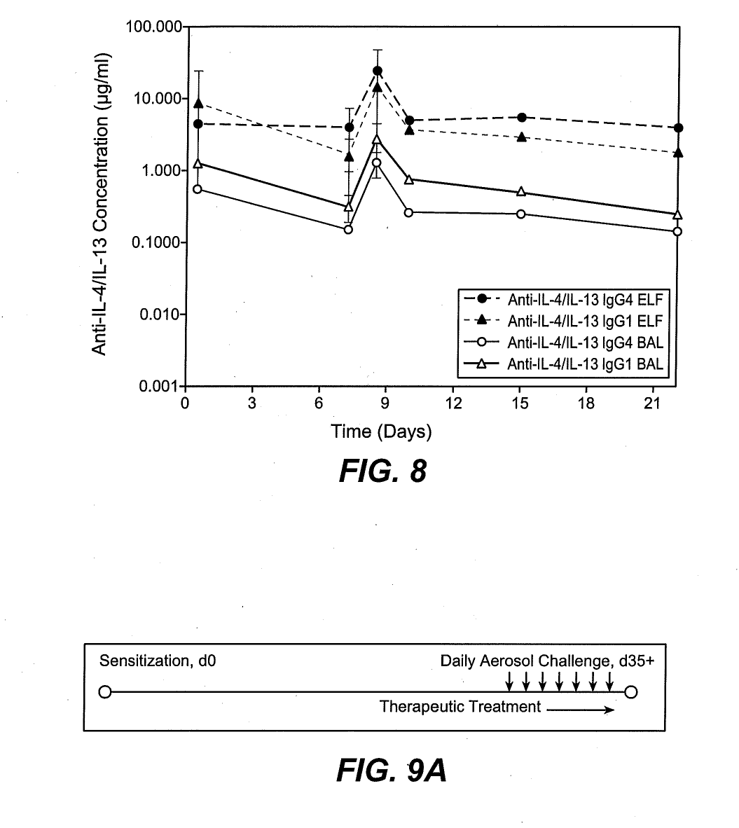

We compared the serum concentrations to epithelial lining fluid (ELF) concentrations of anti-IL-4/IL-13 IgG4 and anti-IL-4/3 IgG1 antibodies following IV administration of 10 mg/kg on Study Days 1 and 8 and a lung challenge with A. suum extract on Study Day 9. IgG concentration values in the ELF were derived by correcting BAL fluid IgG concentration data for dilution inherent to the BAL fluid collection procedure as described, e.g., in Rennard et al., 1986, J. Appl. Physiol., 60(2): 532-538. The serum to lung partitioning of anti-IL-4/IL-13 IgG4 and anti-IL-4/IL-13 IgG1 bispecific antibodies were comparable throughout the length of the study (FIG. 8). Prior to the allergen challenge, ELF concentrations for both antibodies were approximately 1%-4% of IgG serum concentrations, indicating that only a small fraction of the systemic antibody reached the ELF. Inhalation challenge with A. suum on Study Day 9 appeared to result in increased lung partitioning for both antibodies. However, upon normalizing IgG concentrations to albumin concentrations in the ELF and comparing these values to serum IgG concentrations, the data suggested that the increased ELF IgG concentrations following the respiratory challenge were due to non-specific macromolecular vascular leakage induced by the challenge.

### Example 11

### Anti-IL-4, Anti-IL-13, and Anti-IL-4/IL-13 Antibody Efficacy in a

Mouse Allergic Airway Inflammation and Asthma Model

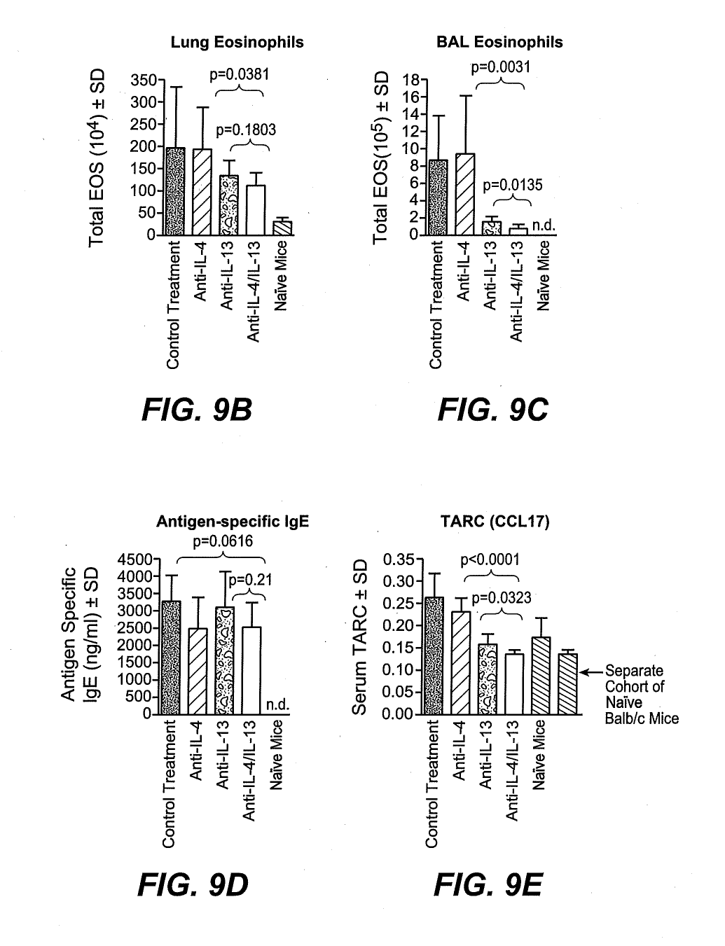

Eight BALB/c mice (Charles River Laboratories) were used in this study. On day 0 all mice were intraperitoneally (IP) immunized with 50 μg trinitrophenyl-ovalbumin (TNP-OVA) in 2 mg alum in 100 μl sterile PBS. Starting on day 35 post immunization, all mice were aerosol challenged daily for 7 consecutive days with 1% TNP-OVA in PBS for 30 minutes via a nebulizer. Starting on day 37, mice were treated daily with monoclonal antibodies (mAbs), administered IP 4 hours prior to each aerosol challenge for 7 days as shown in FIG. 9A.

On day 42, all mice were bled retroorbitally under anesthesia for 200 μl serum terminally (to measure TNP-OVA-specific IgE, IgG1, and antibody serum concentrations achieved during study). Mice were orbitally bled under isoflurane anesthesia to obtain serum samples for TNP-OVA specific immunoglobulin and serum TARC (thymus and activation regulated chemokine) measurements by ELISA. Bronchoalveolar lavage fluid samples were collected for differential counts. Lungs were perfused with cold PBS then analyzed by FACS. Lungs were minced into pieces, then mashed through a metal mash to obtain single cells suspensions, then filtered through vial 0.7 μm nylon filter. Lung samples are resuspended in 5 ml. A fixed volume of cell suspension was added to a fixed concentration of FITC labeled fluorescent beads and analyzed on a flow cytometer, collecting 5000 bead events per sample to obtain cell counts. For quantitative and phenotypic analysis of lungs, 3 million lung cells per sample were stained with fluorochrome-labeled mAbs against surface leukocyte markers (CD44-FTC, CD4-APC, CCR3-Pe and CD4-APC, or CD11c-FITC, CD11b-PE and Gr-1-APC; BD Biosciences, San Jose, Calif.). Samples were run on a BD FACSCalibur (BD, San Jose, Calif.) and analyzed on Flowjo software (Ashland, Oreg.).

The results of that experiment are shown in FIGS. 9B to 9E. Administration of anti-IL-4/IL-13 bispecific antibody suppressed lung eosinophils to a greater extent than anti-IL-4 antibody (p=0.0381), and appeared to suppress lung eosinophils to a greater extent than anti-IL-13 antibody, although the difference did not reach statistical significance (p=0.1803) (FIG. 9B). Similarly, administration of anti-IL-4/IL-13 bispecific antibody suppressed eosinophils in bronchoalveolar lavage fluid to a greater extent than either anti-IL-4 antibody (p=0.0031) or anti-IL-13 antibody (p=0.0135) (FIG. 9C). Administration of either anti-IL-4 antibody or anti-IL-4/IL-13 bispecific antibody appeared to reduce TNP-OVA-specific IgE compared to control treatment, although the results did not reach statistical significance (FIG. 9D). Finally, administration of anti-IL-4/IL-13 bispecific antibody suppressed serum TARC levels to a greater extent than either anti-IL-4 antibody or anti-IL-13 antibody (p<0.0001 and p=0.0323, respectively) (FIG. 9E).

### Discussion

Here we have applied the previously developed knobs-into-holes bispecific antibody platform to generate human IgG1 and human IgG4 bispecific antibodies against the cytokines IL-4 and IL-13. Given the overlapping and unique biologies of IL-4 and IL-13, as well as the activities of anti-IL-13 antibodies in the treatment of moderate-to-severe asthmatics, a bispecific antibody targeting both IL-4 and IL-13 may be an improved therapy over anti-IL-13 for the treatment of asthma. The data presented in Example 11 above is supportive of this hypothesis. Our anti-IL-4/IL-13 bispecific antibody is an extension of the anti-IL-13 antibody lebrikizumab, which showed clinical efficacy in a Phase II study in moderate-to-severe uncontrolled asthma. Since lebrikizumab is a human IgG4 antibody, we used the knobs-into-holes bispecific antibody platform with human IgG4 in order to match the isotype of our anti-IL-4/IL-13 bispecific antibody to that of lebrikizumab.

One of the key differences between human IgG1 and IgG4 isotypes is the CH3 dimer interface, which affects the dimer stability. Differences are driven by position 409. Our results demonstrate that the knobs-into-holes mutations are compatible with Arg409 in the CH3 domain of IgG4, both in terms of expression as half-antibodies as well as assembly into a bispecific antibody. We could not detect any significant differences in the assembly efficiency or in the quality of final antibody material between the two different isotypes.

While the expression of human antibodies of various isotypes is well-established in mammalian cells, there have been fewer attempts to express different human antibody isotypes in E. coli, and thus, the expression of full-length or half antibodies of human IgG4 isotype in E. coli is not as well-documented. Here we demonstrate for these anti-IL-4/IL-13 bispecific antibodies that human IgG4 hemimers can be successfully expressed in large quantities in E. coli cells and assembled into bispecific antibodies as readily as human IgG1 bispecific antibodies.

One of the hallmarks of the knobs-into-holes technology is the retention of the biophysical properties of the monovalent parental antibody in a final bispecific molecule. Both the IgG1 and IgG4 bispecific antibodies retained the target epitope and binding properties of the parental Fab, including high affinity to the IL-4 or IL-13 target cytokine, leading to high potency in in vitro cellular assays.

Pharmacokinetic studies in cynomolgus monkeys demonstrated slow clearance and similar terminal half-lives for both IgG1 and IgG4 bispecific antibodies. In addition, both IgG1 and IgG4 bispecific antibodies partitioned comparably from the serum to the lung at levels that may enable the complete neutralization of pathogenic IL-4 and IL-13 in the lung, which is important for the treatment of asthma. Although the IgG4 bispecific appeared to have a higher rate of ATA compared to the IgG1 bispecific in cynomolgus monkeys, given the small number of animals used in our studies, as well as the lack of a clear relationship between the immunogenicity of humanized antibodies in cynomolgus monkeys vs. humans, we cannot make any conclusions about the relative immunogenicity of our anti-IL-4/IL-13 IgG4 and IgG1 bispecific antibodies in humans. It should be noted, however, that aside from the CDR regions of the antibody Fab's, our bispecific antibodies consist of fully human IgG1 and IgG4 sequences that should exhibit minimal immunogenicity in humans. Thus, the bispecific antibodies that we have generated are good candidates for clinical development for the treatment of asthma as well as IPF and other respiratory disorders. Furthermore, based on the in vivo data presented herein, methods of treating human disorders, such as asthma, IPF and other respiratory disorders, would naturally follow.

Antibodies of different human isotypes can have very different in vitro and in vivo properties resulting from differences in binding to serum complement proteins and Fcγ receptors on immune effector cells (Nirula, A. et al., 2011, Curr Opin Rheumatol 23, 119-124). In particular, antibodies of human IgG1 isotype effectively activate the complement system and engage Fcγ receptors to trigger antibody-dependent cellular cytoxicity (ADCC), whereas antibodies of human IgG4 isotype do not activate the complement system and have reduced ADCC. Importantly, these properties in antibody effector function require antibody glycosylation that is generated during expression in mammalian cells. Antibodies produced in bacterial cells such as E. coli lack antibody effector function (Jung, S. T. et al., 2011, Curr. Opin. Biotechnol. 22, 858-867; Simmons, L. C., et al., 2002, J Immunol Methods 263, 133-147) regardless of isotype, due to a lack of antibody glycosylation. Although the bispecific antibodies produced in this study were produced in E. coli and therefore lacked glycosylation and Fc effector function, the bispecific antibodies described herein may also be produced in mammalian cells. This approach may effectively extend the knobs-into-holes bispecific antibody platform for these antibodies to include fully glycosylated bispecific anti-IL-4/IL-13 of human IgG1 and IgG4 antibody isotypes and may in turn provide a broad range of therapeutic bispecific antibodies with differing effector functions.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

TABLE OF SEQUENCES

| SEQ ID | | |
|---|---|---|
| NO: | Description | Sequence |
| 1 | mu19C11 VH | QIQLVQSGPE LKKPGETVKI SCKASGYTFT DYSMHWMKQA |
| | | PGKGLKWMVW INTETGEPTY ADDFKGRFAF SLETSANTAY |
| | | LKINNLKNED TATYFCARGG IFYGMDYWGQ GTSVTVSS |
| 2 | mu19C11 VL | SIVMTQTPKF LLISAGDRVT ITCKASQSVI NDAAWYQQKP |
| | | GQSPRLLIYY TSHRYTGVPD RFTGSGYGTD FTFTISTVQA |
| | | EDLAVYFCQQ DYTSPWTFGG GTKLEIKR |
| 3 | hu19C11 VH1 | QVQLVQSGAE VKKPGASVKV SCKASGYTFT DYSMHWVRQA |
| | graft | PGQGLEWMVW INTETGEPTY ADDFKGRVTI TRDTSTSTAY |
| | | LELSSLRSED TAVYYCARGG IFYGMDYWGQ GTLVTVSS |
| 4 | hu19C11 VH1.L | QVQLVQSGAE VKKPGASVKV SCKASGYTFT DYSMHWVRQA |
| | graft | PGQGLEWMVW INTETGEPTY ADDFKGRVTI TLDTSTSTAY |
| | | LELSSLRSED TAVYYCARGG IFYGMDYWGQ GTLVTVSS |
| 5 | hu19C11 | QVQLVQSGAE VKKPGASVKV SCKASGYTFT DYSMHWVRQA |
| | VH1.FFL graft | PGQGLEWMVW INTETGEPTY ADDFKGRFTF TLDTSTSTAY |
| | | LELSSLRSED TAVYYCARGG IFYGMDYWGQ GTLVTVSS |
| 6 | hu19C11 VH3 | EVQLVESGGG LVQPGGSLRL SCAASGYTFT DYSMHWVRQA |
| | graft | PGKGLEWVVW INTETGEPTY ADDFKGRFTI SRDNSKNTLY |
| | | LQMNSLRAED TAVYYCARGG IFYGMDYWGQ GTLVTVSS |
| 7 | hu19C11 | EVQLVESGGG LVQPGGSLRL SCAASGYTFT DYSMHWVRQA |
| | VH3.FLA graft | PGKGLEWVVW INTETGEPTY ADDFKGRFTF SLDNSKNTAY |
| | | LQMNSLRAED TAVYYCARGG IFYGMDYWGQ GTLVTVSS |
| 8 | hu19C11 VH3. | EVQLVESGGG LVQPGGSLRL SCAASGYTFT DYSMHWVRQA |
| | LA graft | PGKGLEWVVW INTETGEPTY ADDFKGRFTI SLDNSKNTAY |
| | | LQMNSLRAED TAVYYCARGG IFYGMDYWGQ GTLVTVSS |
| 9 | hu19C11 VH3. | EVQLVESGGG LVQPGGSLRL SCAASGYTFT DYSMHWVRQA |
| | LA.SV graft | PGKGLEWVVW INTETGEPTY ADSVKGRFTI SLDNSKNTAY |
| | | LQMNSLRAED TAVYYCARGG IFYGMDYWGQ GTLVTVSS |
| 10 | hu19C11 VL κ1 | DIQMTQSPSS LSASVGDRVT ITCKASQSVI NDAAWYQQKP |
| | graft | GKAPKLLIYY TSHRYTGVPS RFSGSGSGTD FTLTISSLQP |
| | | EDFATYYCQQ DYTSPWTFGQ GTKVEIKR |
| 11 | hu19C11 VL κ3 | EIVLTQSPAT LSLSPGERAT LSCKASQSVI NDAAWYQQKP |
| | graft | GQAPRLLIYY TSHRYTGIPA RFSGSGSGTD FTLTISSLEP |
| | | EDFAVYYCQQ DYTSPWTFGQ GTKVEIKR |
| 12 | 19C11 HVRH1 | GYTFTDYSMH |
| 13 | 19C11 | VWINTETGEPTYADSVKG |
| | HVRH2.SV | |
| 14 | 19C11 HVRH3 | GGIFYGMDY |
| 15 | 19C11 HVRL1 | KASQSVINDAA |
| 16 | 19C11 HVRL2 | YTSHRYT |
| 17 | 19C11 HVRL3 | QQDYTSPWT |
| 18 | 19C11 HVRH2 | VWINTETGEPTYADDFKG |
| 56 | lebrikizumab | QVTLRESGPA LVKPTQTLTL TCTVSGFSLS AYSVNWIRQP |
| | VH | PGKALEWLAM IWGDGKIVYN SALKSRLTIS KDTSKNQVVL |
| | | TMTNMDPVDT ATYYCAGDGY YPYAMDNWGQ GSLVTVSS |
| 57 | lebrikizumab | DIVMTQSPDS LSVSLGERAT INCRASKSVD SYGNSFMHWY |
| | VL | QQKPGQPPKL LIYLASNLES GVPDRFSGSG SGTDFTLTIS |
| | | SLQAEDVAVY YCQQNNEDPR TFGGGTKVEI KR |
| 19 | lebrikizumab | EVTLRESGPA LVKPTQTLTL TCTVSGFSLS AYSVNWIRQP |
| | VH Q1E | PGKALEWLAM IWGDGKIVYN SALKSRLTIS KDTSKNQVVL |
| | | TMTNMDPVDT ATYYCAGDGY YPYAMDNWGQ GSLVTVSS |
| 20 | lebrikizumab | DIVLTQSPDS LSVSLGERAT INCRASKSVD SYGNSFMHWY |
| | VL M4L | QQKPGQPPKL LIYLASNLES GVPDRFSGSG SGTDFTLTIS |
| | | SLQAEDVAVY YCQQNNEDPR TFGGGTKVEI KR |
| 21 | lebrikizumab | GFSLSAYSVNW |
| | HVRH1 | |
| 60 | lebrikizumab | AYSVN |
| | HVRH1 | |
| | (alternate) | |
| 22 | lebrikizumab | MIWGDGKIVYNSALKS |
| | HVRH2 | |
| 23 | lebrikizumab | DGYYPYAMDN |
| | HVRH3 | |
| 24 | lebrikizumab | RASKSVDSYGNSFMH |
| | HVRL1 | |
| 25 | lebrikizumab | LASNLES |
| | HVRL2 | |
| 26 | lebrikizumab | QQNNEDPRT |
| | HVRL3 | |
| 27 | human IL-4 | MGLTSQLLPP LFFLLACAGN FVHGHKCDIT LQEIIKTLNS |
| | precursor | LTEQKTLCTE LTVTDIFAAS KNTTEKETFC RAATVLRQFY |
| | (Swiss-Prot | SHHEKDTRCL GATAQQFHRH KQLIRFLKRL DRNLWGLAGL |
| | Accession No. | NSCPVKEANQ STLENFLERL KTIMREKYSK CSS |
| | P05112.1) | |
| 28 | human IL-4, | HKCDIT LQEIIKTLNS LTEQKTLCTE LTVTDIFAAS |
| | mature form | KNTTEKETFC RAATVLRQFY SHHEKDTRCL GATAQQFHRH |
| | (without signal | KQLIRFLKRL DRNLWGLAGL NSCPVKEANQ STLENFLERL |
| | sequence) | KTIMREKYSK CSS |
| 29 | human IL-13 | MALLLT TVIALTCLGG FASPGPVPPS TALRELIEEL |
| | precursor | VNITQNQKAP LCNGSMVWSI NLTAGMYCAA LESLINVSGC |
| | (Swiss-Prot | SAIEKTQRML SGFCPHKVSA GQFSSLHVRD TKIEVAQFVK |
| | Accession No. | DLLLHLKKLF REGRFN |
| | P35225.2) | |
| 30 | human IL-13, | SP GPVPPSTALR ELIEELVNIT QNQKAPLCNG |
| | mature form | SMVWSINLTA GMYCAALESL INVSGCSAIE KTQRMLSGFC |
| | (without signal | PHKVSAGQFS SLHVRDTKIE VAQFVKDLLL HLKKLFREGR |
| | sequence) | FN |
| 31 | human IL-13 | LTCLGGFASP GPVPPSTALR ELIEELVNIT QNQKAPLCNG |
| | R130Q mature | SMVWSINLTA GMYCAALESL INVSGCSAIE KTQRMLSGFC |
| | form | PHKVSAGQFS SLHVRDTKIE VAQFVKDLLL HLKKLFREGQ |
| | | FN |
| 32 | cynomolgus | MALLLTMVIA LTCLGGFASP SPVPPSTALK ELIEELVNIT |
| | monkey IL-13 | QNQKAPLCNG SMVWSINLTA GVYCAALESL INVSGCSAIE |
| | precursor | KTQRMLNGFC PHKVSAGQFS SLRVRDTKIE VAQFVKDLLV |
| | (GenBank | HLKKLFREGQ FN |
| | Accession No. | |
| | ABG75889.1) | |
| 33 | cynomolgus | MGLTSQLLPP LFFLLACAGN FVHGHKCDIT LQEIIKTLNS |
| | monkey IL-4 | LTEQKTLCTK LTITDILAAS KNTTEKETFC RAATVLRQFY |
| | precursor | SHHEKDTRCL GATAQQFHRH KQLIRFLKRL DRNLWGLAGL |
| | (Swiss-Prot | NSCPVKEANQ STLENFLERL KTIMREKYSK CSS |
| | Accession No. | |
| | P79339.2); | |
| | mature form is | |
| | amino acids | |
| | 25-153 | |
| 34 | IgG1 T366W | ASTKGPSVFP LAPSSKSTSG GTAALGCLVK DYFPEPVTVS |
| | heavy chain | WNSGALTSGV HTFPAVLQSS GLYSLSSVVT VPSSSLGTQT |
| | constant region | YICNVNHKPS NTKVDKKVEP KSCDKTHTCP PCPAPELLGG |
| | | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLWC LVKGFYPSDI AVEWESNGQP ENNYKTTPPV |
| | | LDSDGSFFLY SKLTVDKSRW QQGNVFSCSV MHEALHNHYT |
| | | QKSLSLSPGK |
| 35 | IgG1 T366S/ | ASTKGPSVFP LAPSSKSTSG GTAALGCLVK DYFPEPVTVS |
| | L368A/Y407V | WNSGALTSGV HTFPAVLQSS GLYSLSSVVT VPSSSLGTQT |
| | heavy chain | YICNVNHKPS NTKVDKKVE KSCDKTHTCP PCPAPELLGG |
| | constant region | PSVFLFPPKP KDTLMISRTP EVTCVVVDVS HEDPEVKFNW |
| | | YVDGVEVHNA KTKPREEQYN STYRVVSVLT VLHQDWLNGK |
| | | EYKCKVSNKA LPAPIEKTIS KAKGQPREPQ VYTLPPSREE |
| | | MTKNQVSLSC AVKGFYPSDI AVEWESNGQP ENNYKTTPPV |
| | | LDSDGSFFLV SKLTVDKSRW QQGNVFSCSV MHEALHNHYT |
| | | QKSLSLSPGK |
| 36 | IgG4 T366W/ | ASTKGPSVFP LAPCSRSTSE STAALGCLVK DYFPEPVTVS |
| | S228P heavy | WNSGALTSGV HTFPAVLQSS GLYSLSSVVT VPSSSLGTKT |
| | chain constant | YTCNVDHKPS NTKVDKRVES KYGPPCPPCP APEFLGGPSV |
| | region | FLFPPKPKDT LMISRTPEVT CVVVDVSQED PEVQFNWYVD |
| | | GVEVHNAKTK PREEQFNSTY RVVSVLTVLH QDWLNGKEYK |
| | | CKVSNKGLPS SIEKTISKAK GQPREPQVYT LPPSQEEMTK |
| | | NQVSLWCLVK GFYPSDIAVE WESNGQPENN YKTTPPVLDS |
| | | DGSFFLYSRL TVDKSRWQEG NVFSCSVMHE ALHNHYTQKS |
| | | LSLSLGK |
| 37 | IgG4 T366S/ | ASTKGPSVFP LAPCSRSTSE STAALGCLVK DYFPEPVTVS |
| | L368A/Y407V/ | WNSGALTSGV HTFPAVLQSS GLYSLSSVVT VPSSSLGTKT |
| | S228P heavy | YTCNVDHKPS NTKVDKRVES KYGPPCPPCP APEFLGGPSV |
| | chain constant | FLFPPKPKDT LMISRTPEVT CVVVDVSQED PEVQFNWYVD |
| | region | GVEVHNAKTK PREEQFNSTY RVVSVLTVLH QDWLNGKEYK |
| | | CKVSNKGLPS SIEKTISKAK GQPREPQVYT LPPSQEEMTK |
| | | NQVSLSCAVK GFYPSDIAVE WESNGQPENN YKTTPPVLDS |
| | | DGSFFLVSRL TVDKSRWQEG NVFSCSVMHE ALHNHYTQKS |
| | | LSLSLGK |
| 38 | hu19C11 IgG4 | EVQLVESGGG LVQPGGSLRL SCAASGYTFT DYSMHWVRQA |
| | T366S/ | PGKGLEWVVW INTETGEPTY ADSVKGRFTI SLDNSKNTAY |
| | L368A/Y407V/ | LQMNSLRAED TAVYYCARGG IFYGMDYWGQ GTLVTVSSAS |
| | S228P heavy | TKGPSVFPLA PCSRSTSEST AALGCLVKDY FPEPVTVSWN |
| | chain | SGALTSGVHT FPAVLQSSGL YSLSSVVTVP SSSLGTKTYT |
| | | CNVDHKPSNT KVDKRVESKY GPPCPPCPAP EFLGGPSVFL |
| | | FPPKPKDTLM ISRTPEVTCV VVDVSQEDPE VQFNWYVDGV |
| | | EVHNAKTKPR EEQFNSTYRV VSVLTVLHQD WLNGKEYKCK |
| | | VSNKGLPSSI EKTISKAKGQ PREPQVYTLP PSQEEMTKNQ |
| | | VSLSCAVKGF YPSDIAVEWE SNGQPENNYK TTPPVLDSDG |
| | | SFFLVSRLTV DKSRWQEGNV FSCSVMHEAL HNHYTQKSLS |
| | | LSLGK |
| 39 | hu19C11 Fight | DIQMTQSPSS LSASVGDRVT ITCKASQSVI NDAAWYQQKP |
| | chain | GKAPKLLIYY TSHRYTGVPS RFSGSGSGTD FTLTISSLQP |
| | | EDFATYYCQQ DYTSPWTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| 40 | lebrikizumab | EVTLRESGPA LVKPTQTLTL TCTVSGFSLS AYSVNWIRQP |
| | Q1E IgG4 | PGKALEWLAM IWGDGKIVYN SALKSRLTIS KDTSKNQVVL |
| | T366W/S228P | TMTNMDPVDT ATYYCAGDGY YPYAMDNWGQ GSLVTVSSAS |
| | heavy chain | TKGPSVFPLA PCSRSTSEST AALGCLVKDY FPEPVTVSWN |
| | | SGALTSGVHT FPAVLQSSGL YSLSSVVTVP SSSLGTKTYT |
| | | CNVDHKPSNT KVDKRVESKY GPPCPPCPAP EFLGGPSVFL |
| | | FPPKPKDTLM ISRTPEVTCV VVDVSQEDPE VQFNWYVDGV |
| | | EVHNAKTKPR EEQFNSTYRV VSVLTVLHQD WLNGKEYKCK |
| | | VSNKGLPSSI EKTISKAKGQ PREPQVYTLP PSQEEMTKNQ |
| | | VSLWCLVKGF YPSDIAVEWE SNGQPENNYK TTPPVLDSDG |
| | | SFFLYSRLTV DKSRWQEGNV FSCSVMHEAL HNHYTQKSLS |
| | | LSLGK |
| 41 | lebrikizumab | DIVLTQSPDS LSVSLGERAT INCRASKSVD SYGNSFMHWY |
| | M4L ligtht | QQKPGQPPKL LIYLASNLES GVPDRFSGSG SGTDFTLTIS |
| | chain | SLQAEDVAVY YCQQNNEDPR TFGGGTKVEI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| 58 | lebrikizumab | QVTLRESGPA LVKPTQTLTL TCTVSGFSLS AYSVNWIRQP |
| | IgG4 T366W/ | PGKALEWLAM IWGDGKIVYN SALKSRLTIS KDTSKNQVVL |
| | S228P heavy | TMTNMDPVDT ATYYCAGDGY YPYAMDNWGQ GSLVTVSSAS |
| | chain | TKGPSVFPLA PCSRSTSEST AALGCLVKDY FPEPVTVSWN |
| | | SGALTSGVHT FPAVLQSSGL YSLSSVVTVP SSSLGTKTYT |
| | | CNVDHKPSNT KVDKRVESKY GPPCPPCPAP EFLGGPSVFL |
| | | FPPKPKDTLM ISRTPEVTCV VVDVSQEDPE VQFNWYVDGV |
| | | EVHNAKTKPR EEQFNSTYRV VSVLTVLHQD WLNGKEYKCK |
| | | VSNKGLPSSI EKTISKAKGQ PREPQVYTLP PSQEEMTKNQ |
| | | VSLWCLVKGF YPSDIAVEWE SNGQPENNYK TTPPVLDSDG |
| | | SFFLYSRLTV DKSRWQEGNV FSCSVMHEAL HNHYTQKSLS |
| | | LSLGK |
| 59 | lebrikizumab | DIVMTQSPDS LSVSLGERAT INCRASKSVD SYGNSFMHWY |
| | light chain | QQKPGQPPKL LIYLASNLES GVPDRFSGSG SGTDFTLTIS |
| | | SLQAEDVAVY YCQQNNEDPR TFGGGTKVEI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| 42 | IL-13 epitope, | ESLINVSG |
| | amino acids 68 | |
| | to 75 of SEQ ID | |
| | NO: 29 (amino | |
| | acids 50 to 57 | |
| | of SEQ ID NO: 30) | |
| 43 | IL-13 epitope, | YCAALESLINVS |
| | amino acids 63 | |
| | to 74 of SEQ ID | |
| | NO: 29 (amino | |
| | acids 45 to 56 | |
| | of SEQ ID NO: 30) | |
| 44 | anti-IL-13 | DIVLTQSPAS LAVSLGQRAT ISCRASQSVS TSSYSYMNWY |
| | mu11H4 VL | QQTPGQPPKL LIKYASNLES GIPARFSGSG SGTDFTLNIH |
| | | PVEEEDTATY YCQHSWEIPY TFGGGT |
| 45 | anti-IL-13 | QVTLKESGPG ILQPSQTLSL TCSFSGFSLS TSDMGVGWIR |
| | mu11H4 VH | QPSGKGLEWL AHIWWDDVKR YNPALKSRLT ISKDTSSSQV |
| | | FLKIASVDTA DTATYYCARI GTNYGYDGLF DYWGQGTTLT |
| | | VSS |
| 46 | anti-IL-13 | DIVMTQSPDS LAVSLGERAT INCRASQSVS TSSYSYMNWY |
| | hu11H4v6 light | QQKPGQPPKL LIKYASNLES GVPDRFSGSG SGTDFTLTIS |
| | chain | SLQAEDVAVY YCQHSWEIPY TFGQGTKVEI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| 47 | anti-IL-13 | EVQLVESGPA LVKPTQTLTL TCTFSGFSLS TSDMGVGWIR |
| | hu11H4v6 | QPPGKALEWL AHIWWDDVKR YNPALKSRLT ISKDTSKNQV |
| | heavy chain | VLTMTNMDPV DTATYYCARI GTNYGYDALF DYWGQGTLVT |
| | | VSSASTKGPS VFPLAPSSKS TSGGTAALGC LVKDYFPEPV |
| | | TVSWNSGALT SGVHTFPAVL QSSGLYSLSS VVTVPSSSLG |
| | | TQTYICNVNH KPSNTKVDKK VEPKSCDKTH TCPPCPAPEL |
| | | LGGPSVFLFP PKPKDTLMIS RTPEVTCVVV DVSHEDPEVK |
| | | FNWYVDGVEV HNAKTKPREE QYNSTYRVVS VLTVLHQDWL |
| | | NGKEYKCKVS NKALPAPIEK TISKAKGQPR EPQVYTLPPS |
| | | REEMTKNQVS LTCLVKGFYP SDIAVEWESN GQPENNYKTT |
| | | PPVLDSDGSF FLYSKLTVDK SRWQQGNVFS CSVMHEALHN |
| | | HYTQKSLSLS PGK |
| 48 | anti-IL-13 | DIVMTQSPDS LAVSLGERAT INCRASQSVS TSSYSYMNWY |
| | hu11H4v6 VL | QQKPGQPPKL LIKYASNLES GVPDRFSGSG SGTDFTLTIS |
| | | SLQAEDVAVY YCQHSWEIPY TFGQGTKVEI K |
| 49 | anti-IL-13 | EVQLVESGPA LVKPTQTLTL TCTFSGFSLS TSDMGVGWIR |
| | hu11H4v6 VH | QPPGKALEWL AHIWWDDVKR YNPALKSRLT ISKDTSKNQV |
| | | VLTMTNMDPV DTATYYCARI GTNYGYDALF DYWGQGTLVT |
| | | VSS |
| 50 | hu11H4v6 | GFSLSTSDMGVG |
| | HVRH1 | |
| 51 | hu11H4v6 | AHIWWDDVKRYNPALKS |
| | HVRH2 | |
| 52 | hu11H4v6 | ARIGTNYGYDALFDY |
| | HVRH3 | |
| 53 | hu11H4v6 | RASQSVSTSSYSYMN |
| | HVRL1 | |
| 54 | hu11H4v6 | YASNLES |
| | HVRL2 | |
| 55 | hu11H4v6 | QHSWEIPYT |
| | HVRL3 | |

1. A multispecific antibody comprising an antigen-binding domain that comprises a first VH/VL unit that specifically binds IL-4 and a second VH/VL unit that specifically binds IL-13, wherein the antibody:
a) inhibits binding of IL-4 to IL-4 receptor alpha (IL-4Rα),
b) inhibits IL-4-induced proliferation of cells in vitro, and/or
c) inhibits IL-13-induced proliferation of cells in vitro.

2. The multispecific antibody of claim 1, wherein the first VH/VL unit comprises HVR-H3 comprising the amino acid sequence of SEQ ID NO: 14, HVR-L3 comprising the amino acid sequence of SEQ ID NO: 17, and HVR-H2 comprising the amino acid sequence of SEQ ID NO: 13 or SEQ ID NO: 18.

3. The multispecific antibody of claim 1 or claim 2, wherein the first VH/VL unit comprises HVR-H1 comprising the amino acid sequence of SEQ ID NO: 12, HVR-H2 comprising the amino acid sequence of SEQ ID NO: 13 or SEQ ID NO: 18, and HVR-H3 comprising the amino acid sequence of SEQ ID NO: 14.

4. The multispecific antibody of any one of claims 1 to 3, wherein the first VH/VL unit comprises HVR-L1 comprising the amino acid sequence of SEQ ID NO: 15, HVR-L2 comprising the amino acid sequence of SEQ ID NO: 16, and HVR-L3 comprising the amino acid sequence of SEQ ID NO: 17.

5. The multispecific antibody of any one of claims 1 to 4, wherein the first VH/VL unit comprises (a) a VH sequence having at least 95% sequence identity to the amino acid sequence of SEQ ID NO: 9; (b) a VL sequence having at least 95% sequence identity to the amino acid sequence of SEQ ID NO: 10; or (c) a VH sequence as in (a) and a VL sequence as in (b).

6. The multispecific antibody of any one of claims 1 to 5, wherein the first VH/VL unit comprises a VH sequence selected from SEQ ID NOs: 1 and 3 to 9.

7. The multispecific antibody of any one of claims 1 to 6, wherein the first VH/VL unit comprises a VL sequence selected from SEQ ID NOs: 2, 10, and 11.

8. The multispecific antibody of claim 1, wherein the first VH/VL unit comprises the VH sequence of SEQ ID NO: 9 and the VL sequence of SEQ ID NO: 10.

9. The multispecific antibody of any one of claims 1 to 8, wherein the second VH/VL unit comprises:
a) HVR-H3 comprising the amino acid sequence of SEQ ID NO: 23, HVR-L3 comprising the amino acid sequence of SEQ ID NO: 26, and HVR-H2 comprising the amino acid sequence of SEQ ID NO: 22; or
b) HVR-H3 comprising the amino acid sequence of SEQ ID NO: 52, HVR-L3 comprising the amino acid sequence of SEQ ID NO: 55, and HVR-H2 comprising the amino acid sequence of SEQ ID NO: 51.

10. The multispecific antibody of any one of claims 1 to 9, wherein the second VH/VL unit comprises:
a) HVR-H1 comprising the amino acid sequence of SEQ ID NO: 21 or the amino acid sequence of SEQ ID NO: 60, HVR-H2 comprising the amino acid sequence of SEQ ID NO: 22, and HVR-H3 comprising the amino acid sequence of SEQ ID NO: 23; or
b) HVR-H1 comprising the amino acid sequence of SEQ ID NO: 50, HVR-H2 comprising the amino acid sequence of SEQ ID NO: 51, and HVR-H3 comprising the amino acid sequence of SEQ ID NO: 52.

11. The multispecific antibody of any one of claims 1 to 10, wherein the second VH/VL unit comprises:
a) HVR-L1 comprising the amino acid sequence of SEQ ID NO: 24, HVR-L2 comprising the amino acid sequence of SEQ ID NO: 25, and HVR-L3 comprising the amino acid sequence of SEQ ID NO: 26; or
b) HVR-L1 comprising the amino acid sequence of SEQ ID NO: 53, HVR-L2 comprising the amino acid sequence of SEQ ID NO: 54, and HVR-L3 comprising the amino acid sequence of SEQ ID NO: 55.

12. The multispecific antibody of any one of claims 1 to 11, wherein the second VH/VL unit comprises:
a) a VH sequence having at least 95% sequence identity to the amino acid sequence of SEQ ID NO: 19;
b) a VL sequence having at least 95% sequence identity to the amino acid sequence of SEQ ID NO: 20;
c) a VH sequence as in (a) and a VL sequence as in (b);
d) a VH sequence having at least 95% sequence identity to the amino acid sequence of SEQ ID NO: 49;
e) a VL sequence having at least 95% sequence identity to the amino acid sequence of SEQ ID NO: 48;
f) a VH sequence as in (d) and a VL sequence as in (e).

13. The multispecific antibody of any one of claims 1 to 12, wherein the second VH/VL unit comprises the VH sequence of SEQ ID NO: 19, 56, or 49.

14. The multispecific antibody of any one of claims 1 to 13, wherein the second VH/VL unit comprises the VL sequence of SEQ ID NO: 20, 57, or 48.

15. The multispecific antibody of any one of claims 1 to 14, wherein the second VH/VL unit comprises the VH sequence of SEQ ID NO: 19 or 56 and the VL sequence of SEQ ID NO: 20 or 57; or the VH sequence of SEQ ID NO: 49 and the VL sequence of SEQ ID NO: 48.

16. The multispecific antibody of any one of claims 1 to 15, wherein the antibody competes for binding to IL-4 with an antibody comprising a VH sequence of SEQ ID NO: 9 and a VL sequence of SEQ ID NO: 10.

17. The multispecific antibody of any one of claims 1 to 16, wherein the antibody competes for binding to IL-13 with an antibody comprising a VH sequence of SEQ ID NO: 19 and a VL sequence of SEQ ID NO: 20, or with an antibody comprising a VH sequence of SEQ ID NO: 49 and a VL sequence of SEQ ID NO: 48.

18. The multispecific antibody of any one of claims 1 to 17, wherein the antibody binds an epitope within amino acids 77 to 89 of SEQ ID NO: 29, or within amino acids 82 to 89 of SEQ ID NO: 29.

19. A multispecific antibody comprising a first VH/VL unit that specifically binds IL-4 and a second VH/VL unit that specifically binds IL-13, wherein the first VH/VL unit comprises the VH sequence of SEQ ID NO: 9 and the VL sequence of SEQ ID NO: 10, and the second VH/VL unit comprises the VH sequence of SEQ ID NO: 19 and the VL sequence of SEQ ID NO: 20.

20. The multispecific antibody of any one of the preceding claims, wherein the antibody is an IgG antibody.

21. The multispecific antibody of claim 20, wherein the antibody is an IgG1 or IgG4 antibody.

22. The multispecific antibody of claim 21, wherein the antibody is an IgG4 antibody.

23. The multispecific antibody of any one of the preceding claims, wherein the antibody comprises a first heavy chain constant region and a second heavy chain constant region, wherein the first heavy chain constant region comprises a knob mutation and the second heavy chain constant region comprises a hole mutation.

24. The multispecific antibody of claim 23, wherein the first heavy chain constant region is fused to the heavy chain variable region portion of a VH/VL unit that binds IL-4.

25. The multispecific antibody of claim 23 or claim 24, wherein the second heavy chain constant region is fused to the heavy chain variable region portion of a VH/VL unit that binds IL-13.

26. The multispecific antibody of claim 23, wherein the first heavy chain constant region is fused to the heavy chain variable region portion of a VH/VL unit that binds IL-13.

27. The multispecific antibody of claim 23 or claim 26, wherein the second heavy chain constant region is fused to the heavy chain variable region portion of a VH/VL unit that binds IL-4.

28. The multispecific antibody of any one of claims 23 to 27, wherein the antibody is an IgG1 antibody and wherein the knob mutation comprises a T366W mutation.

29. The multispecific antibody of any one of claims 23 to 28, wherein the antibody is an IgG1 antibody and wherein the hole mutation comprises at least one, at least two, or three mutations selected from T366S, L368A, and Y407V.

30. The multispecific antibody of any one of claims 23 to 27, wherein the antibody is an IgG4 antibody and wherein the knob mutation comprises a T366W mutation.

31. The multispecific antibody of any one of claims 23 to 27 and 30, wherein the antibody is an IgG4 antibody and wherein the hole mutation comprises at least one, at least two, or three mutations selected from T366S, L368A, and Y407V mutations.

32. The multispecific antibody of claim 23, wherein the antibody comprises a first heavy chain constant region comprising the sequence of SEQ ID NO: 34.

33. The multispecific antibody of claim 23 or claim 32, wherein the antibody comprises a second heavy chain constant region comprising the sequence of SEQ ID NO: 35.

34. The multispecific antibody of claim 23, wherein the antibody comprises a first heavy chain constant region comprising the sequence of SEQ ID NO: 36.

35. The multispecific antibody of claim 23 or claim 34, wherein the antibody comprises a second heavy chain constant region comprising the sequence of SEQ ID NO: 37.

36. A multispecific antibody that binds IL-4 and IL-13, wherein the antibody comprises a first heavy chain comprising the sequence of SEQ ID NO: 38, a first light chain comprising the sequence of SEQ ID NO: 39, a second heavy chain comprising the sequence of SEQ ID NO: 40, and a second light chain comprising the sequence of SEQ ID NO: 41.

37. An isolated antibody that binds IL-4, wherein the antibody comprises:
(a) HVR-H3 comprising the amino acid sequence of SEQ ID NO: 14, HVR-L3 comprising the amino acid sequence of SEQ ID NO: 17, and HVR-H2 comprising the amino acid sequence of SEQ ID NO: 13 or SEQ ID NO: 18; or
(b) HVR-H1 comprising the amino acid sequence of SEQ ID NO: 12, HVR-H2 comprising the amino acid sequence of SEQ ID NO: 13 or SEQ ID NO: 18, and HVR-H3 comprising the amino acid sequence of SEQ ID NO: 14; or
(c) HVR-L1 comprising the amino acid sequence of SEQ ID NO: 15, HVR-L2 comprising the amino acid sequence of SEQ ID NO: 16, and HVR-L3 comprising the amino acid sequence of SEQ ID NO: 17; or
(d) a VH sequence having at least 95% sequence identity to the amino acid sequence of SEQ ID NO: 9; or
(e) a VL sequence having at least 95% sequence identity to the amino acid sequence of SEQ ID NO: 10.

38. The isolated antibody of claim 37, wherein the antibody comprises HVR-H1 comprising the amino acid sequence of SEQ ID NO: 12, HVR-H2 comprising the amino acid sequence of SEQ ID NO: 13 or SEQ ID NO: 18, HVR-H3 comprising the amino acid sequence of SEQ ID NO: 14, HVR-L1 comprising the amino acid sequence of SEQ ID NO: 15, HVR-L2 comprising the amino acid sequence of SEQ ID NO: 16, and HVR-L3 comprising the amino acid sequence of SEQ ID NO: 17.

39. The isolated antibody of claim 37 or claim 38, wherein the antibody comprises a VH sequence having at least 95% sequence identity to the amino acid sequence of SEQ ID NO: 9 and a VL sequence having at least 95% sequence identity to the amino acid sequence of SEQ ID NO: 10.

40. The isolated antibody of any one of claims 37 to 39, wherein the antibody comprises a VH sequence selected from SEQ ID NOs: 1 and 3 to 9.

41. The isolated antibody of any one of claims 37 to 40, wherein the antibody comprises a VL sequence selected from SEQ ID NOs: 2, 10, and 11.

42. An isolated antibody comprising the VH sequence of SEQ ID NO: 9 and the VL sequence of SEQ ID NO: 10.

43. An isolated nucleic acid encoding:
(a) the antibody of any one of claims 1 to 42;
(b) the first VH/VL unit of the multispecific antibody of any one of claims 1 to 34; or
(c) the second VH/VL unit of the multispecific antibody of any one of claims 1 to 34.

44. A host cell comprising the nucleic acid of claim 43.

45. The host cell of claim 44, wherein the host cell is an E. coli cell or a CHO cell.

46. A method of producing an antibody comprising culturing the host cell of claim 44 or claim 45.

47. An immunoconjugate comprising the antibody of any one of claims 1 to 42 and a cytotoxic agent.

48. A pharmaceutical formulation comprising the antibody of any one of claims 1 to 42 and a pharmaceutically acceptable carrier.

49. The antibody of any one of claims 1 to 42 for use as a medicament.

50. The antibody of any one of claims 1 to 42 for use in treating an eosinophilic disorder, an IL-13 mediated disorder, an IL-4 mediated disorder, or a respiratory disorder.

51. The antibody of claim 50, wherein the eosinophilic disorder is selected from asthma, severe asthma, chronic asthma, atopic asthma, atopic dermatitis, allergy, allergic rhinitis, non-allergic rhinitis, contact dermatitis, erythema multiform, bullous skin disease, psoriasis, eczema, rheumatoid arthritis, juvenile chronic arthritis, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angioedema, helminth infections, urticaria, onchocercal dermatitis, eosinophil-associated gastrointestinal disorders, eosinophilic esophagitis, eosinophilic gastritis, eosinophilic gastroenteritis, eosinophilic enteritis, eosinophilic colitis, ulcerative colitis, Whipple's disease, nasal micropolyposis, nasal polyposis, aspirin intolerance, obstructive sleep apnea, Crohn's disease, scleroderma, endomyocardial fibrosis, fibrosis, inflammatory bowel disease, idiopathic interstitial pneumonia, eosinophilic pneumonia, hypersensitivity pneumonitis, goblet cell metaplasia, pulmonary fibrosis, idiopathic pulmonary fibrosis (IPF), pulmonary fibrosis secondary to sclerosis, chronic obstructive pulmonary disease (COPD), hepatic fibrosis, uveitis, cancer, glioblastoma, Hodgkin's lymphoma, and non-Hodgkin's lymphoma.

52. The antibody of claim 50, wherein the IL-13 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma.

53. The antibody of claim 50, wherein the IL-4 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma.

54. The antibody of claim 50, wherein the respiratory disorder is selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis.

55. Use of an antibody of any one of claims 1 to 42 in the manufacture of a medicament for treating an eosinophilic disorder, an IL-13 mediated disorder, an IL-4 mediated disorder, or a respiratory disorder.

56. The use of claim 55, wherein the eosinophilic disorder is selected from asthma, severe asthma, severe asthma, chronic asthma, atopic asthma, atopic dermatitis, allergy, allergic rhinitis, non-allergic rhinitis, contact dermatitis, erythema multiform, bullous skin disease, psoriasis, eczema, rheumatoid arthritis, juvenile chronic arthritis, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angioedema, helminth infections, urticaria, onchocercal dermatitis, eosinophil-associated gastrointestinal disorders, eosinophilic esophagitis, eosinophilic gastritis, eosinophilic gastroenteritis, eosinophilic enteritis, eosinophilic colitis, ulcerative colitis, Whipple's disease, nasal micropolyposis, nasal polyposis, aspirin intolerance, obstructive sleep apnea, Crohn's disease, scleroderma, endomyocardial fibrosis, fibrosis, inflammatory bowel disease, idiopathic interstitial pneumonia, eosinophilic pneumonia, hypersensitivity pneumonitis, goblet cell metaplasia, pulmonary fibrosis, idiopathic pulmonary fibrosis (IPF), pulmonary fibrosis secondary to sclerosis, chronic obstructive pulmonary disease (COPD), hepatic fibrosis, uveitis, cancer, glioblastoma, Hodgkin's lymphoma, and non-Hodgkin's lymphoma.

57. The use of claim 55, wherein the IL-13 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma.

58. The use of claim 55, wherein the IL-4 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma.

59. The use of claim 55, wherein the respiratory disorder is selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis.

60. A method of treating an individual with an eosinophilic disorder comprising administering to the individual an effective amount of an antibody of any one of claims 1 to 42.

61. The method of claim 60, wherein the eosinophilic disorder is selected from asthma, severe asthma, chronic asthma, atopic asthma, atopic dermatitis, allergy, allergic rhinitis, non-allergic rhinitis, contact dermatitis, erythema multiform, bullous skin disease, psoriasis, eczema, rheumatoid arthritis, juvenile chronic arthritis, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angioedema, helminth infections, urticaria, onchocercal dermatitis, eosinophil-associated gastrointestinal disorders, eosinophilic esophagitis, eosinophilic gastritis, eosinophilic gastroenteritis, eosinophilic enteritis, eosinophilic colitis, ulcerative colitis, Whipple's disease, nasal micropolyposis, nasal polyposis, aspirin intolerance, obstructive sleep apnea, Crohn's disease, scleroderma, endomyocardial fibrosis, fibrosis, inflammatory bowel disease, idiopathic interstitial pneumonia, eosinophilic pneumonia, hypersensitivity pneumonitis, goblet cell metaplasia, pulmonary fibrosis, idiopathic pulmonary fibrosis (IPF), pulmonary fibrosis secondary to sclerosis, chronic obstructive pulmonary disease (COPD), hepatic fibrosis, uveitis, cancer, glioblastoma, Hodgkin's lymphoma, and non-Hodgkin's lymphoma.

62. The method of claim 60, wherein the IL-13 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma.

63. The method of claim 60, wherein the IL-4 mediated disease is selected from atopic dermatitis, allergic rhinitis, asthma, fibrosis, inflammatory bowel disease, Crohn's disease, lung inflammatory disorders, pulmonary fibrosis, idiopathic pulmonary fibrosis (IPF), chronic obstructive pulmonary disease (COPD), hepatic fibrosis, cancer, glioblastoma, and non-Hodgkin's lymphoma.

64. The method of claim 60, wherein the respiratory disorder is selected from asthma, allergic asthma, non-allergic asthma, bronchitis, chronic bronchitis, chronic obstructive pulmonary disease (COPD), emphysema, cigarette-induced emphysema, airway inflammation, cystic fibrosis, pulmonary fibrosis, allergic rhinitis, and bronchiectasis.

65. The method of any one of claims 60 to 64, further comprising administering to the individual a TH2 pathway inhibitor.

66. The method of claim 65, wherein the TH2 pathway inhibitor inhibits at least one target selected from ITK, BTK, IL-9, IL-5, IL-13, IL-4, OX40L, TSLP, IL-25, IL-33, IgE, IL-9 receptor, IL-5 receptor, IL-4 receptor alpha, IL-13receptoralpha1, IL-13receptoralpha2, OX40, TSLP-R, IL-7Ralpha, IL17RB, ST2, CCR3, CCR4, CRTH2, FcepsilonR1, FcepsilonRII/CD23, Flap, Syk kinase; CCR4, TLR9, CCR3, IL5, IL3, and GM-CSF.

67. The method of any one of claims 60 to 66, wherein the individual is suffering from moderate to severe asthma.

68. The method of any one of claims 60 to 66, wherein the individual is suffering from idiopathic pulmonary fibrosis.
